(19) United States
(12) Patent Application Publication
(10) Pub. No.: US 2010/0221763 A1
(43) Pub. Date: Sep. 2, 2010

(54) Specific Acceptors for Transferases to Saccharides and Method for Obtaining and Using Same

(75) Inventors: Khushi L. Matta, Williamsville, NY (US); E.V. Chandrasekaran, Northbrook, IL (US)

Correspondence Address: MICHAEL L. DUNN, SIMPSON & SIMPSON, PLLC, 5555 MAIN STREET, WILLIAMSVILLE, NY, 14221, US

(73) Assignee: Health Research, Inc, Buffalo, NY (US)

(21) Appl. No.: 12/083,537

(22) PCT Filed: Oct. 17, 2006
(86) PCT No.: PCT/US06/40518

§ 371 Date: May 18, 2010

Related U.S. Application Data
(60) Provisional application No. 60/727,359, filed on Oct. 17, 2005.

(51) Int. Cl. C12Q 1/48; C07H 3/06

(52) U.S. Cl. 435/15; 536/123.1

(57) ABSTRACT

A method for determining substrates specific for a transferase enzyme selected from the group consisting of glycosyltransferases and sulfotransferases. The method includes the steps of:
a) selecting a substrate to be tested for specificity for sulfotransferases and for glycosyltransferases, from tri, tetra or penta saccharide compounds containing at least one →GlcNAcβ1 saccharide that may be substituted with MeO—, EtO—, Allyl-O, or N₃— and terminating in →3GalNAcα-OR, →3GlcNAcβ-OR or →3Galα-O—R where R is alkyl, aryl, azido or allyl all of 1 through 8 carbon atoms where R may be substituted with halo, —OH, lower alkyl, —SO₃, or —NO₂;
b) combining the substrate separately with each glycosyltransferase or sulfo-transferase in a series, for transfer of a particular glycosyl or sulfo moiety, to a saccharide, in conjunction with a donor compound for the particular moiety, where the moiety is radio labeled, in a buffered aqueous solution of about pH of about 6 to about 7, to form incubation mixtures;
c) incubating the incubation mixtures at a temperature of from about 18 to about 40 degrees Celcius for from about 30 minutes to about four hours;
d) testing the resulting incubation mixtures of each separate glycosyltransferase or sulfotransferase for reaction product comprising a chemical combination of substrate and particular moiety to determine effectiveness of each separate glycosyltransferase or sulfotransferase in transferring the particular moiety to the substrate; and
e) comparing the effectiveness of each glycosyltransferase or sulfotransferase in transferring the particular moiety to determine whether a particular glycosyltranferase or sulfotransferase acts to transfer the particular moiety to the substrate when the other glycosyltransferases or sulfotransferases do not do so, thus determining whether the substrate acts specifically with a particular glycosyltransferase or sulfotransferase.

The invention also includes a method for the specific detection of a specific transferase for a specific moiety selected from sulfotransferases and glycosyltransferases.

### STATEMENT REGARDING FEDERALLY SPONSORED RESEARCH OR DEVELOPMENT

This work was supported by the NIH (USA) Grant CA35329. The United States Government may have certain rights in this invention

### BACKGROUND OF THE INVENTION

This invention relates to acceptors for transferases, more particularly glycosyltranferases and sulfotransferases and the screening and use of both known and novel acceptors (substates) for the detection and quantification of specific transferases.

An important area of cancer research is to search for biomarkers for a very early detection of the disease, thus avoiding the cancer spread and metastasis. Prostate specific antigen (PSA), the biomarker of prostate cancer [Chu, 1997; Brawer, 1999; Milford et al., 2001] performs poorly when used to differentiate prostate cancer from benign prostatic hyperplasia [Bonn, 2002]. Pancreatic cancer is another leading cause of cancer related deaths in Western countries [Magnani et al., 1983; Mangray & King, 1998; Lillemoe et al., 2000]. Monoclonal antibodies for CA19-9, a mucin-associated sialyl Lewis^{a}, along with other serum tumor markers have been proposed for diagnosis and follow up of these diseases [Magnani et al., 1983]. CA 125 is used world wide as a marker for detection and follow up of ovarian cancer with some limitations [Bast et al., 2002]. At present, biomarkers unique for cancers of breast, colon, stomach, lung and other organs are practically non-existent.

While considerable advances have been made in genomic and proteomic approaches to cancer research, few reports describe cancer glycomics. As an example, genechips have been used to measure the expression of cellular glycosyltransferases mRNA in human colon tissue However, mRNA expression may not reflect enzyme levels, which in turn may not predict glycosylation patterns.

Changes in glycosylation are nevertheless commonly observed in human carcinomas [Dennis et al., 1999; Brockhausen, 1999] and may contribute to the malignant phenotype downstream of certain oncogenic events. Carbohydrate antigens associated with cancer can be divided into three major classes: (i) Glycosphingolipids of ganglio and globo series. (ii) Lacto series type 1 (Galβ1,3GlcNAc) and type 2 (Galβ1,4GlcNAc) chains carrying sialyl Le^{a} and sialyl Le^{x} determinants that can occur in both tumor-associated glycolipids and glycoproteins. (iii) Tumor-associated glycoprotein antigens may be either N- or O-Glycans. Recent studies emphasize the importance of understanding the structure of carbohydrates present in tumor tissue to meet at least two clinical needs. In one aspect, antibodies against unique carbohydrate in tumor-associated antigens, especially those secreted into blood, may provide new and better biomarkers. Secondly, glycans uniquely expressed on cancer cells provide targets for novel cancer therapy. The levels of glycosyltransferase activities are altered in cancer cells and hence target structures can be selected on an enzymatic basis considering the specificity of the particular enzymes involved in the assembly of these complex carbohydrate antigens. Detailed structural determination of carbohydrate chains of already known tumor associated antigens has shown the existence of both N- and O-glycan chains in CA125 [Wong et al., 2003], a glycosylation difference between prostate cancer cell PSA and normal PSA [Okada et al., 2001; Peracaula et al., 2003a; Prakash & Robbins, 2000; Ohyama et al., 2004], and a difference in N-glycan chains of human pancreatic ribonuclease isolated from healthy pancreas vs. pancreatic cancer [Peracaula et al., 2003b]. It has become apparent from these structural analyses of glycans that major changes are at the outer end of carbohydrate chains in cancer-associated antigens. This has prompted the use of lectins to detect the outer end glycosylation changes of tumor-associated glycoconjugates. The changes in the activities of glycosyltransferases reflect the structures of glycans expressed by cancer cells. Thus a knowledge of glycosytranferase activities in cancer cells can reveal valuable information about the structures likely to be expressed by cancer cells and allow the identification and selection of better carbohydrate epitopes for immunohistochemical detection of cancer. Tumor associated mucins from breast, pancreas and colon carcinoma are known to contain mucin core 2 branched structures [Fukuda, 1996; Hollingsworth & Swanson, 2004]. The target epitopes that can occur as part of core 2 O-linked glycoproteins associated with cancers can be identified by measuring the activities of various glycosyltransferases on defined acceptors. Mucin core 2 structure Galβ1, 3(GlcNAcβ1, 6) GalNAcα-Ser/Thr serves as a carrier of selectin ligands [Li et al., 1996]. The malignant potential of a tumor cell is yet to be defined in terms of the relative amounts of sialyl Lewis^{x} and sialyl Lewis^{a}, which serve as the selectin ligands, to that of sialyl Tn and Tn epitopes expressed by the same tumor cell [Komminoth et al., 1991; Piller et al., 1991]. As cancer-associated specific changes in the cellular repertoire of glycosyltransferases define the final glycosylation profiles of glycoconjugates [Müller & Hanisch, 2002], the present study was undertaken to examine the patterns of various glycosyltransferase activities in several cancer cells and then verifying these patterns in two tumor tissues.

The structural variability of glycans is dictated by tissue-specific regulation of glycosyltransferase genes, the availability of suguar nucleotides and competition between enzymes for acceptor intermediates during glycan elongation. The studies of Müller & Hanisch [2002] on recombinant MUC1 probe expressed in four breast cancer cell lines indicate that epigenetic parameters like the rate of Golgi passage and the topology of the protein substrate within the Golgi compartment are less important with respect to the final glycosylation profiles than the cellular repertoire of glycosyltransferases. Several glycosyltransferases and Gal3-O-Sulfotransferases are capable of elongating mucin core 2 tetrasaccharide as well as the Globo backbone unit. Such action of these enzymes could lead to a complexity of cancer associated terminal glycan structures, as illustrated in FIG. 1. Müller & Hanisch [2002] expressed a MUC1 fusion protein MFP6 in the breast cancer cell lines ZR-75-1, MDA-MB-231, MCF-7 and T47D and studied the O-glycans of the fusion proteins after releasing by hydrazinolysis and then labeling with 2-amino benzamide.

Sialic acids play an important role in a variety of biological processes, like cell-cell communication, cell-substrate interaction, adhesion, maintenance of serum glycoproteins in the circulation, and protein targeting. Cell surface sialic acid occurs in a variety of structures, which change in a regulated manner during development, differentiation, and oncogenic transformation. Very little is known about the regulation of cell surface sialyl oligosaccharides, which are involved in cellular processes. Sialic acids occur at the terminal positions of the carbohydrate groups of glycoproteins and glycolipids. The transfer of sialic acid from CMP-Sia to these positions is catalyzed post-translationally by a family of glycosyltransferases called sialyltransferases.

To date 19 different human sialyltransferase genes have been identified that catalyze the transfer of sialic acid from CMP-Sia into an α2,3-, α2,6- or α2,8-linkage to the terminal position of carbohydrates on glycoproteins and glycolipids. Analysis of the protein sequences of sialyltransferases reveals, as for most glycosyltransferases, a topological feature characteristic for Type 2 transmembrane proteins. All sialyltransferases cloned to date contain two conserved sialylmotifs in the catalytic part of the enzyme. A large sialylmotif of approximately 48 amino acids is termed the “L (large)-sialylmotif” which participates in the binding of the donor substrate CMP-Neu5Ac and a smaller sequence of about 23 amino acids towards the C-terminal is termed the “S (short)-sialylmotif” which participates in the binding of both the donor and the acceptor. Also, a very short sequence, the “VS-motif”, further down towards the C-terminal and comprising only 3-5 amino acids has been identified as a conserved sequence of importance for the enzymatic activity.

Distinct cell-surface carbohydrates are expressed in tissue- and cell-specific manners during development and in adulthood. Aberrations in cell-surface carbohydrates often are associated with malignant transformation and other pathological condition. Among various cell-type-specific carbohydrates, sialyl Lewis x, NeuNAcα2→3Galβ1→4(Fucα1→3)GlcNAcβ→R, is expressed on neutrophils, monocytes and certain T-lymphocytes and plays a key role in the recruitment of leukocytes. E- and P-selectin expressed on activated endothelial cells capture these leukocytes through binding to sialyl Lewis x, allowing them to roll, which leads to extravasation of leukocytes at inflammatory sites. Lymphocyte circulation is directed by interaction between L-selectin on lymphocytes and the sulfated form of sialyl Lewis x present on L-selectin receptors that are restricted to high endothelial venules. Such an initial interaction leads to extravasation of lymphocytes from the intravascular compartment to the lymphatic compartment.

The amounts of sialyl Lewis x and sialyl Lewis a, NeuNAcα2→3Galβ1→3(Fucα1→4)GlcNAc→R, which also has been shown to bind to E-selectin are increased significantly in tumor cells such as carcinoma and leukemia. In breast and colonic carcinoma patients, the presence of sialyl Lewis x and sialyl Lewis a is correlated with poor prognosis. These results strongly suggest that blood-borne tumor cells may use a carbohydrate-selectin (or a selectin-related molecule) interaction when tumor cells adhere to the endothelia at metastatic sites.

Tumor dissemination may thus be inhibited by use of sialyl Lewis x oligosaccharides as antagonists. Lewis x is recognized by CD94 on NK cells. Apparently, D94 binds more efficiently to sialyl Lewis x densely attached to carrier glycans than to sialyl Lewis x sparsely attached to carrier glycans. By contrast, sparsely or moderately attached sialyl Lewis x facilitates tumor metastasis. Thus a difference in sialyl Lewis x expression leads to entirely different biological consequences.

Cancer-associated specific changes in the cellular repertoire of glycosyltransferases apparently thus define the final glycosylation profiles of glycoconjugates. The structural analyses of glycans indicate that major changes are at the outer end of carbohydrate chains in cancer-associated antigens. The changes in the activities of glycosyltransferase reflect the altered structures of glycans expressed by cancer cells. Thus a knowledge of glycosyltransferase activities in cancer cells can reveal valuable information about the structures likely to be expressed by cancer cells and allow the identification and selection of better carbohydrate epitopes for development of monoclonal antibodies which can prove to be better biomarkers for cancer. For gaining this knowledge, it is mandatory that specific and sensitive acceptors should become available for measuring the level of various glycosyltransferase activities in cancer sera, cancer cells and tumor tissues. The above important aspect has prompted the development of well-defined acceptors for measuring sialyltransferase and glycosyltransferase activities in presence of other sialyl and glycosyltransferase activities in accordance with the present invention.

Further, an important area of cancer research is to search for biomarkers for a very early detection of the disease, thus avoiding the cancer spread and metastasis. Prostate specific antigen (PSA), the biomarker of prostate cancer [Chu, 1997; Brawer, 1999; Milford et al., 2001] performs poorly when used to differentiate prostate cancer from benign prostatic hyperplasia [Bonn, 2002]. Pancreatic cancer is another leading cause of cancer related deaths in Western countries [Magnani et al., 1983; Mangray & King, 1998; Lillemoe et al., 2000]. Monoclonal antibodies for CA19-9, a mucin-associated sialyl Lewis^{a}, along with other serum tumor markers have been proposed for diagnosis and follow up of these diseases [Magnani et al., 1983]. CA 125 is world wide used marker for detection and follow up of ovarian cancer with some limitations [Bast et al., 2002]. At present, biomarkers unique for cancers of breast, colon, stomach, lung and other organs are practically non-existent.

Changes in glycosylation are commonly observed in human carcinomas [Dennis et al., 1999; Brockhausen, 1999] and may contribute to the malignant phenotype downstream of certain oncogenic events. Carbohydrate antigens associated with cancer can be divided into three major classes: (i) Glycosphingolipids of ganglio and globo series. (ii) Lacto series type 1 (Galβ1,3GlcNAc) and type 2 (Galβ1,4GlcNAc) chains carrying sialyl Le^{a} and sialyl Le^{x} determinants that can occur in both tumor-associated glycolipids and glycoproteins. (iii) Tumor-associated glycoprotein antigens may be either N- or O-Glycans. Recent studies emphasize the importance of understanding the structure of carbohydrates present in tumor tissue for two clinical needs. In one aspect, antibodies against unique carbohydrate in tumor-associated antigens, especially those secreted into blood, may provide new and better biomarkers. Secondly, glycans uniquely expressed on cancer cells provide targets for novel cancer therapy. The levels of glycosyltransferase activities are altered in cancer cells and hence target structures can be selected on an enzymatic basis considering the specificity of the particular enzymes involved in the assembly of these complex carbohydrate antigens. Detailed structural determination of carbohydrate chains of already known tumor associated antigens has shown the existence of both N- and O-glycan chains in CA125 [Wong et al., 2003], a glycosylation difference between prostate cancer cell PSA and normal PSA [Okada et al., 2001; Peracaula et al., 2003a; Prakash & Robbins, 2000; Ohyama et al., 2004], and a difference in N-glycan chains of human pancreatic ribonuclease isolated from healthy pancreas vs. pancreatic cancer [Peracaula et al., 2003b]. It has become apparent from these structural analyses of glycans that major changes are at the outer end of carbohydrate chains in cancer-associated antigens. This has prompted the use of lectins to detect the outer end glycosylation changes of tumor-associated glycoconjugates. The changes in the activities of glycosyltransferases reflect the structures of glycans expressed by cancer cells. Thus a knowledge of glycosytranferase activities in cancer cells can reveal valuable information about the structures likely to be expressed by cancer cells and allow the identification and selection of better carbohydrate epitopes for immunohistochemical detection of cancer. Tumor associated mucins from breast, pancreas and colon carcinoma are known to contain mucin core 2 branched structures [Fukuda, 1996; Hollingsworth & Swanson, 2004]. The target epitopes that can occur as part of core 2 O-linked glycoproteins associated with cancers can be identified by measuring the activities of various glycosyltransferases on defined acceptors. Mucin core 2 structure Galβ1, 3(GlcNAcβ1, 6) GalNAcα-Ser/Thr serves as a carrier of selectin ligands [Li et al., 1996]. The malignant potential of a tumor cell is yet to be defined in terms of the relative amounts of sialyl Lewis^{x} and sialyl Lewis^{a}, which serve as the selectin ligands, to that of sialyl Tn and Tn epitopes expressed by the same tumor cell [Komminoth et al., 1991; Piller et al., 1991]. As cancer-associated specific changes in the cellular repertoire of glycosyltransferases define the final glycosylation profiles of glycoconjugates [Milner & Hanisch, 2002], the present study was undertaken to examine the patterns of various glycosyltransferase activities in several cancer cells and then verifying these patterns in two tumor tissues. Using the present invention, enabled the identification of an association of signature carbohydrate structures with individual cancers.

### BRIEF DESCRIPTION OF THE SEVERAL VIEWS OF THE DRAWINGS

FIG. 1A) A composite picture of Biogel P₂ Column-elution profiles for [¹⁴C] sialylated products arising from the action of cloned α2,3(O)ST on mucin core 2 acceptors Galβ1,4GlcNAcβ1,6(Galβ1,3)GalNAcα-O-Bn (∘—∘—∘), 3-O-SulfoGalβ1,4GlcNAcβ1,6 (Galβ1.3)GalNAcα-O-Bn (——) and 3-O-MeGalβ1,4GlcNAcβ1,6(Galβ1,3)GalNAcα-O-Bn ( - - - - - - ). CMP[¹⁴C]NeuAc emerged from the Biogel P2 Column at 56-64 mL with a peak at 60 mL; B) Thin layer chromatography [Solvent System: 1-Propanol/NH₄OH/H₂O v/v (12/2/5)] followed by autoradiography for [¹⁴C] sialyl products isolated from the following acceptors as shown in FIG. 1A: 3-O-MeGalβ1,4GlcNAcβ1,6(Galβ1,3)GalNAcα-O-Bn (lane 1), Galβ1,4GlcNAcβ1,6(Galβ1,3)GalNAcα-O-Bn (lane 2) and 3-O-SulfoGalβ1,4GlcNAcβ1,6(Galβ1,3) GalNAcα-O-Bn (lane 3).

FIG. 2 Proposed biosynthetic pathways for mucin core 2 heptasaccharide with sialyl Le^{x (PSGL-}1), core 2 hexasaccharide containing GalNAc Le^{x} (potential selectin ligand), potential Siglec ligands containing 6′-sialyl, 6-sulfo LacNAc, and core 2 oligosaccharide with 6-Sulfo sialyl Le^{x} (GlyCAM-1). Key: □=α-GalNAc; ▪=GlcNAc; =Gal; ♦=NeuAc; □=Fuc and S=Sulfo group.

FIG. 3 Sialyltransferase activity at varying concentration of mucin core 2 based tetrasaccharide acceptors for cloned α2,6(N)ST (panel A), α2,3(N)ST (panel B), α2,3(O)ST (panel C) and LNCaPα2,3(O)ST (panel D). The acceptor used in A and B are Galβ1,4GlcNAcβ1,6(Galβ1,3)GalNAcα-O-Bn (——) and 4-O-MeGalβ1,4GlcNAcβ1,6(Galβ1,3)GalNAcα-O-Bn (∘—∘—∘). In panel C and D the acceptors are Galβ1,4GlcNAcβ1,6(Galβ1,3)GalNAcα-O-Bn (——) and Galβ1,4GlcNAcβ1,6(4-O-MeGalβ1,3)GalNAcα-O-Bn (∘—∘—∘). Dotted lines in panel C denote enzymatic reaction data when donor CMP-NeuAc was used at the higher concentration in the reaction mixture.

FIG. 4 Thin layer chromatography of non-radioactive acceptors lane 1. 3-O-MeGalβ1,3(GlcNAcβ1,6) GalNAcα-O-Bn, lane 2. Fucα1,2Galβ1, 3(GlcNAcβ1, 6)GalNAcα-O-Bn, lane 3. 3-O-SulfoGalβ1,3 (GlcNAcβ1,6)GalNAcα-O-Bn and lane 4. Galβ1,3(GlcNAcβ1,6)GalNAcα-O—Al in two solvent systems 1-propanol/NH₄OH/H₂O (12/2/5) in Panel A (developed once) and CHCl₃/CH₃OH/H₂O (5/4/1) in Panel C (developed twice). Thin layer chromatography of [9 ³H] sialyl compounds obtained by the action of cloned α2,3(O)ST on the above acceptors (1. to 4. in the same sequence). The solvent in Panel B was 1-propanol/NH₄OH/H₂O (12/2/5) (developed once) and in Panel D it was CHCl₃/CH₃OH/H₂O (5/4/1) (developed twice).

FIG. 5 WGA-agarose chromatography of [9-³H] sialylated product obtained following the action of cloned α21,3(O)ST on: A. 3-O-SulfoGalβ1,3(GlcNAcβ1,6)GalNAcα-O-Bn, B. 3-O-MeGalβ1,3(GlcNAcβ1,6) GalNAcα-O-Bn, C. Galβ1,3(GlcNAcβ1,6)GalNAcα-O—Al, D. Fucα1,2Galβ1,3(GlcNAcβ1,6)GalNAcα-O-Bn, E. A mixture of the product of B above incubated with and without β-N-acetylhexosaminidase, F. Product of C above incubated with β-N-acetylhexosaminidase, G. Product of D above incubated with β-N-acetylhexosaminidase. In all runs, elution was performed with 0.5M GlcNAc after the 10^{th} fraction.

FIG. 6 Cloned α2,3(O)ST activity at varying concentrations of acceptors containing modified Galactosyl residue linked β1,3 to α-GalNAc. A. Acceptor concentration versus enzyme activity for D-Fucβ1,3(GlcNAcβ1,6)GalNAcα-O-Bn (——), 4-O-MeGalβ1,3GalNAcα-O-Bn (∘—∘—∘), 4-FGalβ1,3GalNAcα-O-Bn (▴—▴—▴) and D-Fucβ1,3GalNAcα-O-Bn (□—□—□). B. Lineweaver-Burke plot for data in panel A. C. Acceptor concentration versus enzyme activity for Galβ1,3GalNAcβ1,3Galα-O-Me (□—□—□), 3-O-SulfoGalβ1,3GalNAcβ1,3Galα-O-Me (▴—▴—▴), D-Fucβ1,3GalNAcβ1,3Galα-O-Me (∘—∘—∘) and 3-O-SulfoD-Fucβ1,3GalNAcβ1,3Galα-O-Me (——). D. Lineweaver-Burke plot for data in panel C. E. Acceptor concentration versus enzyme activity for Fucα1,2Galβ1,3(GlcNAcβ1,6)GalNAcα-O-Bn (CMP-NeuAc: 15 μM) (∘—∘—∘), Fucα1,2Galβ1,3 (GlcNAcβ1,6)GalNAcα-O-Bn (CMP-NeuAc: 150 μM) (□—□—□), 3-O-MeGalβ1,3(GlcNAccβ1,6) GalNAcα-O-Bn (CMP-NeuAc: 0.3 μM) (□—□—□) and 3-O-MeGalβ1,3(6-O-Me)GalNAcα-O-Bn (CMP-NeuAc: 0.3 μM) (▪—▪—▪). Lineweaver-Burke plot for data in panel E.

FIG. 7 Fragmentation of A. compound 6; B. compound 23; C. compound 7 and D. compound 18 in Table VI under the same CID condition of ESI-MS. Fragments are annotated according to the scheme proposed by Domon and Costello (42).

FIG. 8 MS² spectra of double charged molecular species [M-2H]²⁻ at m/z 604 derived from A. compound 6; B. compound 23; C. compound 7 and D. compound 18 in Table VI.

FIG. 9 MS³ spectra of A. Y′₂ ion at m/z 917 derived from compound 6; B. Y₃ ion or Y′₂ ion at m/z 917 derived from compound 23 or compound 7 and C. Y₃ ion at m/z 913 derived from compound 18 in Table VI.

FIG. 10 shows various potential competition for glysosyl and sulfotransferases acting on mucin core 2 and Globo backbone.

### BRIEF DESCRIPTION OF THE INVENTION

The present invention enables an association of signature carbohydrate structures with individual cancers.

Well-defined acceptors thus provide tools to examine enzyme activities that extend results available using other experimental methods. The unique acceptors, of the present invention, for these enzymes can be used in presence of another enzyme that transfers sugar at other position of the acceptor. In accordance with the present invention, it has thus been found that modified analogs of acceptors with fluoro or methyl groups can be better and specific acceptors for transferase enzymes. For example, we have observed that MeO-2Galβ1→3GlcNAcβ1-O-Benzyl is a novel acceptor for STIV as this compound is not prone to α2,3 sialyltransferase STI,II and α2,6-sialyltransferase activities.

The invention thus includes a method for determining substrates specific for a transferase enzyme selected from the group consisting of glycosyltransferases and sulfotransferases. The method includes the steps of:

a) selecting a substrate to be tested for specificity for sulfotransferases and for glycosyltransferases, from tri, tetra or penta saccharide compounds containing at least one →GlcNAcβ1 saccharide that may be substituted with MeO—, EtO—, Allyl-O, or N₃— and terminating in →3GalNAcα-OR, →3GlcNAcβ-OR or →3Galα-O—R where R is alkyl, aryl, azido or ally all of 1 through 8 carbon atoms where R may be substituted with halo, —OH, lower alkyl, —SO₃, or —NO₂;
b) combining the substrate separately with each glycosyltransferase or sulfo-transferase in a series, for transfer of a particular glycosyl or sulfo moiety, to a saccharide, in conjunction with a donor compound for the particular moiety, where the moiety is radio labeled, in a buffered aqueous solution of about pH of about 6 to about 7, to form incubation mixtures;
c) incubating the incubation mixtures at a temperature of from about 18 to about 40 degrees Celcius for from about 30 minutes to about four hours;
d) testing the resulting incubation mixtures of each separate glycosyltransferase or sulfotransferase for reaction product comprising a chemical combination of substrate and particular moiety to determine effectiveness of each separate glycosyltransferase or sulfotransferase in transferring the particular moiety to the substrate; and.
e) comparing the effectiveness of each glycosyltransferase or sulfotransferase in transferring the particular moiety to determine whether a particular glycosyltranferase or sulfotransferase acts to transfer the particular moiety to the substrate when the other glycosyltransferases or sulfotransferases do not do so, thus determining whether the substrate acts specifically with a particular glycosyltransferase or sulfotransferase.

The invention also includes a method for the specific detection of a specific transferase for a specific moiety selected from sulfotransferases and glycosyltransferases including the steps of:

a) selecting a substrate specific for the transferase from tri, tetra or penta saccharide compounds containing at least one pre-terminal saccharide that may be substituted with MeO—, EtO—, Allyl-O, or N₃— and terminating in →3GalNAcα-OR, →3Galα-O—R or →3GlcNAcβ-OR where R contains 1 to 12 carbon atoms and is alkyl, aryl, azido or allyl and R may be substituted with halo, —OH, lower alkyl, —SO₃ or —NO₂;
b) combining the substrate with a sample to be tested for the specific transferase in conjunction with a donor compound for the particular moiety, in a buffered aqueous solution of about pH 6 to about pH 7, to form an incubation mixture;
c) incubating the incubation mixture at a temperature of from about 18 to about 40 degrees Celcius for from about 30 minutes to about four hours; and
d) testing the resulting incubation mixture for reaction product comprising a chemical combination of substrate and particular moiety to determine the presence of the particular transferase in the sample.

Steps a) through d) are commonly repeated with different concentrations of substrate to determine the minimum concentration of substrate required to maximize quantity of obtained combination of substrate and particular moiety and the determined minimum concentration is correlated with quantity of the specific transferase in the sample.

The invention also includes the newly discovered substrates Galβ1→4GlcNAcβ1→6(MeO-4Galβ1 →4)GalNAcα→O—R; MeO-Galβ1→4GlcNAcβ1→6(Galβ1→3)GalNAcα→O—R; Galβ1→4GlcNAcβ1→6(F-4Galβ1→3) GalNAcα→O—R; and F-4Galβ1→4GlcNAcβ1→6(Galβ1→3)GalNAcα→O—R where R contains 1 to 12 carbon atoms and is alkyl, aryl, azido, or allyl, that may be substituted with halo, —OH, —SO₃, or —NO₂.

### DETAILED DESCRIPTION OF THE INVENTION

In accordance with the invention it has been found that many of the substrates used in the invention terminates in →3GalNAcα-OR and many of the substrates used in the invention also contain at least one of Galβ1, DFucβ1 or an additional →GlcNAcβ1 where the Galβ1, DFucβ1 or additional →GlcNAcβ1 may be substituted with MeO—, EtO—, Allyl-O, or N₃—. The transferase is commonly selected from sialyltransferases, fucosyltransferases, N-acetylglucosaminyl transferases, N-acetylgalactosaminyl transferases, and galactosyltransferases.

The incubation temperature in the method is usually from about 35 to about 40° C. and is most commonly about 37° C.

The substrate may be a novel oligosaccharide compound containing 3 to 5 linked monosaccharides containing at least one →GlcNAcβ1 saccharide that may be substituted with MeO—, EtO—, Allyl-O, or N₃— and terminating in →3GalNAcα-OR where R is alkyl, aryl or allyl all of 1 through 8 carbon atoms where R may be substituted with halo, —OH, —SO₃, or —NO₂ and further containing at least one of Galβ1, DFucβ1 or an additional →GlcNAcβ1 where the Galβ1, DFucβ1 or additional →GlcNAcβ1 may be substituted with MeO—, EtO—, Allyl-O, or N₃—.

More specifically the novel compound may be selected from the group consisting of Galβ1→4GlcNAcβ1→6(MeO-4Galβ1→3)GalNAcα→O—R; MeO-4Galβ1→4GlcNAcβ1→6(Galβ1→3)GalNAcα→O—R; Galβ1→4GlcNAcβ1→6(F-4Galβ1→3) GalNAcα→O—R; F-4Galβ1→4GlcNAcβ1→6(Galβ1 →3)GalNAcα→O—R; MeO-3Galβ1→3(GlcNAcβ1→6)GalNAcα→O—R; Galβ1→3(GlcNAcβ1→6) GalNAcα→O—R; Galβ1→3(GlcNAcβ1→6) GalNAcαO—R; D-Fucβ1→3GalNAcβ1→3Galα-O-methyl; and GalNAcβ1→4GlcNAcβ1→6(GalNAcβ1→3)GalNAcα→O—R.

The substrate specific for the transferase is usually a tri, tetra or penta saccharide compound containing at least one pre-terminal saccharide that may be substituted with MeO—, EtO—, Allyl-O, or N₃— and terminating in →3GalNAcα-OR, →3Galα-O—R or →3GlcNAcβ-OR where R contains 1 to 12 carbon atoms and is alkyl, aryl or allyl and R may be substituted with halo, —OH, lower alkyl, —SO₃, azido or —NO₂. R is commonly aryl that may be substituted with —OH, halo, or lower alkyl. R is usually, allyl, benzyl, methyl, azido or nitrophenyl and most commonly benzyl.

The substrate usually contains at least one GalNAcβ1 saccharide and most commonly terminates in →3GalNAcα-OR. The substrate also commonly contains at least one of Galβ1, DFucβ1 or an additional →GlcNAcβ1 where the Galβ1, DFucβ1 or additional →GlcNAcβ1 may be substituted with MeO—, EtO—, Allyl-O, or N₃—.

The substrate, is most commonly unsubstituted, with the exception of saccharide linkages, NAc groups and terminal groups. With the exception of saccharide linkages, the Galβ1, DFucβ1 or additional →GlcNAcβ1 are usually unsubstituted.

Following are specific examples of specific substrates, their corresponding specific transferases and appropriate donor compound for providing the transferred moiety.

1. The specific substrate is Galβ1→4GlcNAcβ1→6(MeO-3Galβ1→3)GalNAcα→O—R, the specific transferase is sialyl transferase IV (ST IV) and the donor compound is cytidine-5′ monophospate-Nacetylneuraminic acid (CMP-sialic acid).

2. The specific substrate is MeO-Galβ1→4GlcNAcβ1→6(Galβ1→3)GalNAcα→O—R, the specific transferase is sialyl transferase I/II (ST I/II) and the donor compound is cytidine-5′ monophospate-Nacetylneuraminic acid (CMP-sialic acid).

3. The substrate is Galβ1→4GlcNAcβ1→6(F-3Galβ1→3)GalNAcα→O—R, the specific transferase is sialyl transferase IV (ST IV) and the donor compound is cytidine-5′ monophospate-Nacetylneuraminic acid (CMP-sialic acid).

4. The substrate is F-3Galβ1→4GlcNAcβ1→6(Galβ1→3)GalNAcα→O—R, the specific transferase is sialyl transferase I/II (ST I/II) and the donor compound is cytidine-5′ monophospate-N-acetylneuraminic acid (CMP-sialic acid).

5. The substrate is Galβ1→4GlcNAcβ1→6(MeO-4Galβ1→4)GalNAcα→O—R, the specific transferase is α1,4 Nacetylglucosaminyl transferase II (α1,4-GNTII) and the donor compound is uridine diphosphate N-acetylglucosamine (UDP-GlcNAc).

6. The substrate is (MeO-4Galβ1→4)GlcNAcβ1→6(Galβ1→3)GalNAcα→O—R, the specific transferase is α1,4 N-acetylglucosaminyl transferase I (α1,4-GNTI) and the donor compound is uridine diphosphate N-acetylglucosamine (UDP-GlcNAc).

7. The substrate is Galβ1 →4GlcNAcβ1 →6(F-4Galβ1 →3)GalNAcα→O—R, the specific transferase is α1,4 N-acetylglucosaminyl transferase II (α1,4-GNTII) and the donor compound is uridine diphosphate N-acetylglucosamine (UDP-GlcNAc).

8. The substrate is F-4Galβ1→4GlcNAcβ1→6(Galβ1→3)GalNAcα→O—R, the specific transferase is α1→4 N-acetylglucosaminyl transferase I (α1,4-GNTI) and the donor compound is uridine diphosphate N-acetylglucosamine (UDP-GlcNAc).

9. The substrate is NeuAcα2→3Galβ1→4GlcNAcβ1→6(MeO-3Galβ1→3)GalNAcα→O—R, the specific transferase is the sulfotranserferase, galactose 6 sulfotransferase I (Gal-6-SulfoTI) and the donor compound is 3′ phosphoadenosine 5′ phosphosulfate (PAPS).

10. The substrate is MeO-3Galβ1→4GlcNAcβ1→6(NeuAcα2→3)Galβ1→3GalNAcα→O—R, the specific transferase is the sulfotranserferase, galactose 6 sulfotransferase II (Gal-6-SulfoTII) and the donor compound is 3′ phosphoadenosine 5′ phosphosulfate (PAPS).

11. The substrate is Galβ1→4MeO-3GlcNAcβ1→6(GalNAcβ1→3)GalNAcα→O—R, the specific transferase is α1,2 L-fucosyltransferase I (α1,2-L-FT I) and the donor compound is guanosine diphosphate fucose (GDP-fucose).

12. The substrate is GalNAcβ1→4(MeO-3GlcNAc)β1→6(Galβ1→3)GalNAcα→O—R, the specific transferase is α1,2 L-fucosyltransferase II (α1,2-L-FT II) and the donor compound is guanosine diphosphate fucose (GDP-fucose).

13. The substrate is GalNAcβ1→4GlcNAcβ1→6(GalNAcβ1→3)GalNAcα→O—R, the specific transferase is α1,3 L-fucosyltransferase (α1,3-L-FT) and the donor compound is guanosine diphosphate fucose (GDP-fucose).

14. The substrate is Galβ1→3(GlcNAcβ1→6) GalNAcα→O—R, the specific transferase is β1→3/4 N-acetyl galactosaminyl transferase (β1→3/4 GalNAc-T) and the donor compound is uridine diphosphate galactosamine (UDP-GalNAc).

15. The substrate is MeO-3Galβ1→3(GlcNAcβ1→6)GalNAcα→O—R, the specific transferase is an αX N-acetyl glucosminyl transferase (αXGlcNAc-T) and the donor compound is uridine diphosphate N-acetylglucosamine (UDP-GlcNAc).

16. The substrate is 2-O-MeGalβ1→4GlcNAcβ-O-benzyl, the specific transferase is sialyl transferase IV (Gal3ST IV) and the donor compound is cytidine-5′ monophospate-N-acetylneuraminic acid (CMP-sialic acid).

17. The substrate is 4-O-MeGalβ1→4GlcNAcβ-O-benzyl, the specific transferase is sialyl transferase IV (Gal3ST IV) and the donor compound is cytidine-5′ monophospate-N-acetylneuraminic acid (CMP-sialic acid).

18. The substrate is D-Fucβ1→3GalNAcβ1→3Galα-O-methyl, the specific transferase is sialyl transferase I/II (Gal3ST I/II) and the donor compound is cytidine-5′ monophospate-N-acetylneuraminic acid (CMP-sialic acid).

19. The substrate is 2-O-MeGalβ1→4GlcNAcβ-O-benzyl, the specific transferase is sialyl transferase I/II (ST I/II) and the donor compound is cytidine-5′ monophospate-N-acetylneuraminic acid (CMP-sialic acid).

20. The substrate is 2-O-MeGalβ1→4GlcNAcβ-O-benzyl, the specific transferase is sialyl transferase I/II (ST I/II) and the donor compound is cytidine-5′ monophospate-N-acetylneuraminic acid (CMP-sialic acid).

21. The substrate is 2-O-MeGalβ1→4GlcNAcβ-O-benzyl, the specific transferase is sialyl transferase I/II (ST I/II) and the donor compound is cytidine-5′ monophospate-N-acetylneuraminic acid (CMP-sialic acid).

22. The substrate is 2-O-MeGalβ1→4GlcNAcβ-O-benzyl, the specific transferase is sialyl transferase I/II (ST I/II) and the donor compound is cytidine-5′ monophospate-N-acetylneuraminic acid (CMP-sialic acid).

Sialic acids are key determinants in many carbohydrates involved in biological recognition. As a further aspect of the present invention, We studied the acceptor specificities of three cloned sialyltransferases (STs) α2,3(N)ST, α2,3(O)ST and α2,6(N)ST, and another α2,3(O)ST present in prostate cancer cell LNCaP towards mucin core-2 tetrasaccharide [Galβ1,4GlcNAcβ1,6(Galβ1,3)GalNAcα-O-Bn] and Globo [Galβ1,3GalNAcβ1,3Galα-O-Me] structures containing sialyl-, fucosyl-, sulfo-, methyl- or fluoro-substituents by identifying the products by eletrospray ionization tandem mass spectral analysis and other biochemical methods. The Globo precursor was an efficient acceptor for both α2,3(N)ST and α2,3(O)ST whereas only α2,3(O)ST used its deoxy analog [D-Fucβ1,3GalNAcβ1,3-Gal-α-O-Me]; 2-O-MeGalβ1,3GlcNAc and 4-OMeGalβ1,4GlcNAc were specific acceptors for α2,3(N)ST. Other major findings in this study were: i) α2,3 sialylation of β1,3Gal in mucin core 2 can proceed even after α1,3fucosylation of β1,6-linked LacNAc; ii) sialylation of β1,3 Gal must precede the sialylation of β1,4Gal for favorable biosynthesis of mucin core 2 compounds; iii) α2,3 sialylation of 6-O-SulfoLacNAc moiety in mucin core 2 (eg. GlyCAM-1) is facilitated when β1,3Gal has already been α2,3 sialylated; iv) α2,6(N)ST was absolutely specific for the β1,4 Gal in mucin core 2. Either α1,3 fucosylation or 6-O-Sulfation of the GlcNAc moiety reduced the activity. Sialylation of β1,3Gal in addition to 6-O-sulfation of GlcNAc moiety abolished the activity; v) prior α2,3-sialylation or 3-O-sulfation of β1,3Gal would not affect α2,6-sialylation of Galβ1,4GlcNAc of mucin core 2; vi) 3- or 4-Fluoro substituent in β1,4Gal resulted in poor acceptors for the cloned α2,6(N)ST and α2,3(N)ST whereas 4-Fluoro or 4-OMe Galβ1,3GalNAcα was a good acceptor for cloned α2,3(O)ST; vii) 4-O-methylation of β1,4Gal abolished the acceptor ability towards α2,6(N)ST but increased the acceptor efficiency considerably towards α2,3(N)ST; viii) just like LNCaPα1,2-FT and Gal-3-O-sulfotransferase T2, the cloned α2,3(N)ST which modifies terminal Gal in Galβ1,4GlcNAc also efficiently utilizes the terminal β1,3Gal in the Globo backbone. Utilization of C-3 block compounds such as 3-O-sulfo-Galβ1,3GalNAcβ1,3Galα-OMe as acceptors by cloned α2,3(O)ST and analyses of the resulting products by lectin chromatography and mass spectrometry indicate that α2,3(O)ST is capable of attaching NeuAc to another position in C-3 substituted β1,3Gal.

Sialyl groups of cell surface and secreted glycoconjugates (1,2) can act as binding targets for viruses, bacteria, parasites and toxins (3,4) and also can recognize mammalian selectin and Siglec lectins (3). Sialyltransferases (STs) attach sialic acid to galactose via α2,3 and α2,6 glycosidic bonds, and to GalNAc via α2,6 linkages and also form polysialic acid via α2,8-linkage.

Siglecs in contrast to the majority of immunoglobulin superfamily members, which recognize protein ligands, bind to specific sialylated glycans (5-7). The carbohydrate recognition motif of Siglec-2, Siglec-5 and Siglec-7 have been identified as NeuAcα2,6Galβ1,4Glc (8-10). A role for α2,6 linked sialic acid as a modulator of immune cell interaction in B-cells is evident from the finding that B-cell activation with polyclonal anti-IgM and interferon-γ, leads to specific loss of α2,6 linked sialic acid, and this creates access to costimulatory molecules for the cell surface (11).

Selectins are another family of sialic acid recognizing adhesion molecules, containing amino terminal C-type lectin domains (12-15). They recognize sialyl Lewis-x or sialyl Lewis-a motifs (16-18). Our studies confirmed the importance of α2,3 linked sialic acid and α1,3/4 linked fucose for selectin recognition of ligands, and they demonstrate the role of the mucin core 2 structure in enhancing L- and P-selectin binding to carbohydrates (19). We have also demonstrated the contribution of the NeuAcα2,3Galβ1,3GalNAcα-sequence of mucin core 2 structure in modulating L- and P-selectin binding function (20). Finally, we also showed a unique carbohydrate sequence lacking sialic acid namely GalNAcβ1,4(Fucα1,3)GlcNAcβ-(GalNAc Lewis x) that could act as a ligand for E-selectin (20). As compared to NeuAcα2,3Galβ1,4(Fucα1,3) GlcNAcβ-O-Me (sialyl Lewis-x), we found GalNAcβ1,4(Fucα1,3)GlcNAcβ1,6(NeuAcα2,3Galβ1,3) GalNAcα-O-Me to be a 5-6 fold better inhibitor of L- and P-Selectin binding.

Previously, we used various modified disaccharides as acceptors for studying the specificities of a purified porcine liver α2,3ST acting on Galβ1,3GalNAcα- and a cloned α2,3ST utilizing Galβ1,3/4GlcNAcβ-(21). In view of the importance of sialic acid residues in various biological recognition phenomena, we decided to study in detail the acceptor substrate specificities of three clonal sialyltransferases and also another sialyltransferase that is strictly specific for Galβ1,3GalNAcα-sequence. A variety of mucin core 2 and Globo based synthetic compounds and related simple structures were utilized as acceptors in order to gain insight into the sialylation sequence in the biosynthesis of complex carbohydrate ligands. The study also reveals a novel action of cloned α2,3(O)sialyltransferase in catalyzing the attachment of sialic acid to another position of Galactose in Galβ1,3GalNAc moiety containing a C-3 substituent.

The prostate carcinoma cell line, LNCaP, was grown in RPMI 1640 supplemented with 10% fetal bovine serum and antibiotics (penicillin, streptomycin and amphotericin B) under conditions recommended by American Type Culture Collection (Manassas, Va.). The cells were homogenized with 0.1M Tris-Maleate pH6.3 containing 2% Triton X-100 using a Dounce all glass hand-operated homogenizer. The homogenate was centrifuged at 16,000 g for 1 h at 4° C. Protein concentration in supernatant was measured using the BCA assay (Pierce Biotech, Inc., Rockford, Ill.), with BSA as the standard. The supernatant was adjusted to 5 mg protein/ml by adding the necessary amount of extraction buffer and then stored frozen at −20° C. until use. 10 μl aliquot of this extract was used in assays run in duplicate.

Cloned Sialyltransferases:

Rat recombinant α2,3(O)ST, α2,3(N)ST and α2,6(N)ST were purchased from Calbiochem and stored at either −20° C. or −70° C. as recommended by the supplier. Suitable aliquots were diluted with 1.0 ml of 0.1M NaCacodylate buffer pH 6.0 containing 2% Triton CF-54 and 2% BSA and used in the experiments; α2,3(O)ST and α2,6(N)ST as diluted above were found to retain full activity for at least three months when stored frozen at −20° C. α2,3(N)ST was diluted just before use in the experiment. 10 μl aliquots of the diluted enzymes were used in the assays run in duplicate.

Synthetic Acceptors:

The synthesis of several compounds that are used as acceptors in the present study have been published (20, 22-24). The synthesis of acceptors containing the Globo H precursor structure, namely, Galβ1,3GalNAcβ1,3Galα-O—Al; Galβ1,3GalNAcβ1,3Galα-O-Me; D-Fucβ,1,3GalNAcβ1,3Galα-O-Me; 3-O-SulfoGalβ1,3GalNAcβ1,3Galα-O-Me and 3-O-Sulfo-D-Fucβ1,3GalNAcβ1,3Galα-O-Me and mucin core 2 tetrasaccharides containing 4-O-Me group and complex structural units will be published elsewhere. Mass spectrometry analysis of many of these compounds is presented in this manuscript. The synthesis of mucin core 2 tetrasaccharides containing 3-F group has been reported (25).

Macromolecular and Natural Acceptors:

Acrylamide copolymer of Galβ1,3GalNAcα-O—Al synthesized by the procedure of Horejsi et. al. (26) and Fetuin triantennary asialo glycopeptide were available from earlier studies of this laboratory (21, 27-29).

Assay of Sialyltransferases:

The incubation mixtures run in duplicate contained 0.1M NaCacodylate buffer pH6.0, the acceptor (typically at 7.5 mM, or as indicated in some experiments), CMP-[9 ³H] NeuAc (typically 0.2 μci; 20 μci/nmol or as indicated in some experiments) and the enzyme in a total volume of 20 μl. The control incubation mixtures contained everything except the acceptor. Incubation was carried out for 2 h at 37° C. The enzymatic transfer of [9-³H] NeuAc to a typical acceptor was linear for 2 h and less than 30% CMP-[9-³H] NeuAc was utilized. Chromatography, using either Dowex-1-Formate, Sep-Pak C18 or Biogel P2, was applied to separate radioactive product from un-reacted [9-³H] NeuAc as described below. The values for the duplicate runs did not vary by more than 5%.

The radioactive products from neutral allyl and methyl glycosides as well as non-glycosides were measured by fractionation on Dowex-1-Formate (Bio-Rad: AG-1X8; 200-400 mesh; format form) as follows: The incubation mixture was diluted with 1.0 ml water and passed through AG-1-formate column (1.0 ml bed volume in a Pasteur pipet which had been washed with 5 ml of 2M Formic acid followed by 10 ml water). The column was washed twice with 1.0 ml water after the entry of the sample and then eluted with 3.0 ml of 0.1M NaCl. The radioactivity present in water and 0.1M NaCl eluates were measured separately using 3a70 scintillation cocktail (Research Products International, Mount Prospect, Ill.) and a Beckman LS6500 scintillation counter. The CPM values were corrected by subtracting the blank CPM. Any radioactivity present in the water eluate (as noticed in the case of some methylglycosides) was added to the corresponding CPM value of 0.1M NaCl eluate. The radioactive products from sulfated and/or sialylated methylglycosides were also measured by the above procedure, only the elution of the AG-1-Formate column was continued further with 3.0 ml of 0.2M NaCl for achieving a complete elution of the radioactive product, a correction being made as before by subtracting the corresponding blank values.

The radioactive products from benzylglycosides and monosialylated benzylglycosides were measured by hydrophobic chromatography on Sep-Pak C18 cartridge (Waters, Milford, Mass.) and eluting the product with 3.0 ml methanol (30). The radioactivity was determined by liquid scintillation as above.

Radioactive sialylation products from sulfated benzylglycosides were quantitated by fractionation of the reaction mixture on a BioGel P2 column, since these products were not elutable from AG-1-Formate column even by 0.2M NaCl, and they did not bind to Sep-Pak C18. For such work, a Biogel P2 column (Fine Mesh; 1.0×116.0 cm) was used with 0.1M pyridine acetate pH5.4 as the eluent at room temperature. The effluent fractions were monitored for radioactivity and the first peak containing radioactivity was collected, lyophilized to dryness, dissolved in 200 μl of water and stored frozen at −20° C. for thin layer chromatography (TLC) and other experimentation.

Thin Layer Chromatography:

TLC was carried out on Silica gel GHLF (250 μm, scored 20×20 cm; Analtech, Newark Del.). The solvent systems 1-propanol/NH₄OH/H₂O (v/v 12/2/5) and CHCl₃/CH₃OH/H₂O (v/v5/4/1) were used. The acceptor compounds were located on the plates by spraying with sulfuric acid in ethanol and heating at 100° C. The radioactive products were located by scraping 0.5 cm width segments of silica gel and soaking in 2.0 ml water in vials followed by liquid scintillation counting. Fluorography of the TLC plates was done at −70° C. using Bio-Max MS Film (Eastman Kodak) after spraying the TLC plates with Enhance (Dupont).

WGA-, PNA- and RCA-1-Agarose Affinity Chromatography:

A column of 5 ml bed volume of lectin-agarose (Vector Lab, Burlingame, Calif.) was employed using 10 mM Hepes pH7.5 containing 0.1 mM CaCl₂, 0.01 mM MnCl₂ and 0.1% NaN₃ as the running buffer. Fractions of 1.0 ml were collected. The bound material was then eluted with 0.5M GlcNAc after the 10^{th} fraction in the case of WGA-agarose, or with 0.2M Galactose after the 12^{th} fraction for the RCA-1-agarose and PNA-agarose columns in the same buffer as recommended by the manufacturer. Fractionation was done at room temperature.

Mass Spectral Analysis of Enzymatically Sialylated Compounds:

Reaction mixtures contained 0.3 μmol acceptor, 0.2 μmol CMP-NeuAc, 1.0 μCi CMP [9-³H] NeuAc, 30 μg BSA, 0.1M Na Cacodylate pH 6.0 and 20 milliunit of the cloned sialyltransferase in a total volume of 100 μl and incubated for 18 h at 37° C. After incubation, the reaction mixture was diluted with 1.0 ml water and then subjected to column chromatography on a Biogel P₂ column (1.0×116.0 cm) as described above. The first peak containing the radioactive product was collected, lyophilized to dryness, dissolved in 200 μl water and stored frozen at −20° C. until mass spectral analysis.

MSⁿ experiments (31) were carried out with an Esquire-LC, Bruker-HP ion trap (Bremen, Germany). Samples at 10 pM/μL in MeOH were infused into the electrospray source via a 50 μm id fused silica capillary using a syringe pump at a flow rate of 5 μl/min. Nitrogen was used as the nebulizing gas (at 5-6 psi) and also as the drying gas (5-7 l/min at 200° C.). The potentials of the spray needle, capillary exit, and skimmer were set to ±4,000, 90-150, and 25-50 V, respectively. Helium was the buffer/collision gas. For each spectrum 100-500 scans were averaged. The fragmentation amplitude was varied between 0.8 and 1.5 depending on the experiment design. The fragmentation amplitude was 1.15 for MS² and 1.20 for MS³. The fragmentation time was 40 ms. Typically, the low mass cutoff was set at slightly less than ⅓rd the precursor m/z value.

All monosialyated or monosulfated oligosaccharides were found to lose protons easily to yield [M-H]⁻ ion in negative mode. The oligosaccharides bearing both sialyl and sulfate groups were detected as double charged ions [M-2H]²⁻ in negative mode. The monosialyated or monosulfated oligosaccharides yielded singly charged doubly sodiated ions [M-H+2Na]⁺ in positive mode. The oligosaccharides bearing both sialyl and sulfate group were detected as doubly charged quadruply sodiated ions [M-2H+4Na]²⁺ in positive mode. In contrast to the positive mode, the negative mode could easily avoid the interference of ions from impurity and this yielded higher signal intensity of the product ions. However, the sodiated adduct ions detected in the positive mode provided additional information of the molecular weight, and they confirmed the results got from negative polarity electrospray mass spectra.

Sialyltransferase Assay

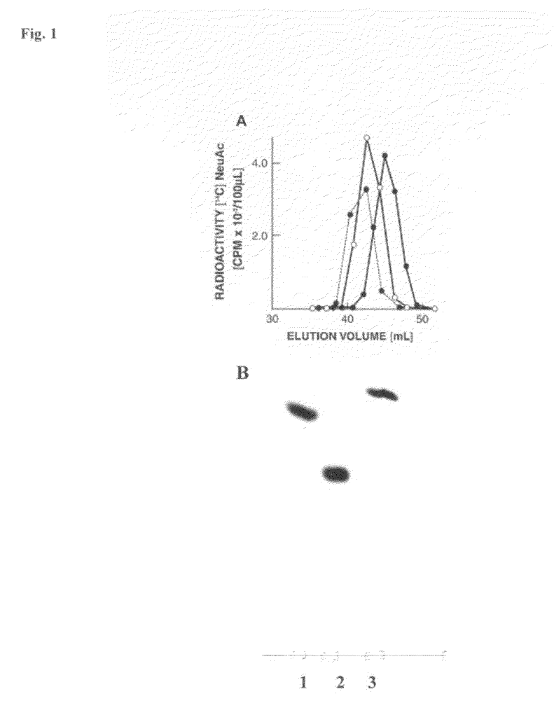

Sialyltransferase activity was measured using methods which we have documented elsewhere (21). In addition to these published protocols, in the current manuscript, the BioGel P2 column was used to separate [9-³H] labeled benzyl glycosides that were both sulfated and sialylated from unreacted CMP-[9-³H]NeuAc (FIG. 1). In this figure, sample Biogel P2 elution profiles are shown for three radioactive products along with corresponding autoradiographs of these products following TLC.

Modified Disaccharides and GloboH Based Acceptors can Distinguish Between the Cloned Enzymes (Table I, Part A)

The cloned α2,3(N)ST and α2,6(N)ST displayed preference for Galβ1,4GlcNAcβ-O—Al rather than Galβ1,3GalNAcα-O—Al, though α2,3(N)ST showed 24.1% activity on the latter compound; α2,3(O)ST and LNCaP STs, on the other hand, showed higher specificity for Galβ1,3GalNAcα-O—Al compared to Galβ1,4GlcNAcβ-O—Al and also acted efficiently on Galβ1,3(GlcNAcβ1,6)GalNAcα-O—Al.

We examined various modifications of the above disaccharides to determine if unique acceptors could be identified. We observed that α2,6(N)ST utilized Galβ1,4GlcNAcβ-O—Al and 2-O-MeGalβ1,4GlcNAc as acceptors (100.0% and 90.1% active), but had very low activity towards 3-O-MeGalβ1,4GlcNAc (8.7% active), Galβ1,3GlcNAcβ-O—Al (3.0%), 2-O-MeGalβ1,3GlcNAc (3.7%) and 2-O-MeGalβ1,3GlcNAcβ-O-Bn (0%). α2,3(O)ST exhibited either very low or negligible activity with acceptors 2-O-MeGalβ1,3GlcNAc (2.9%), 2-O-MeGalβ1,4GlcNAc (0.2%) and 3-O-MeGalβ1,4GlcNAc (3.6%). α2,3(N)ST was the only enzyme that acted efficiently to sialylate 2-O-MeGalβ1,3GlcNAc (115.3%), suggesting that this may be a unique acceptor for this enzyme. It also acted efficiently on Galβ1,3GlcNAcβ-O—Al (123.1%) and 2-O-Me-Galβ1,4GlcNAc (60.0%).

The GloboH precursor Galβ1,3GalNAcβ1,3Galα-O-Me and its deoxy analog D-Fucβ1,3GalNAcβ1,3-Gal-O-Me could be used to distinguish between the enzymes. α2,3(O)ST utilized both the GloboH precursor (120.8% active) and its deoxy analog (115.6%). α2,3(N)ST on the other hand acted efficiency on the Globo H precursor (106.8% active) but not its deoxy analog (3.1% active) indicating that the C-6 hydroxyl group of the β1,3-linked Gal in Globo H precursor is absolutely required for this enzyme activity. In contrast, α2,6(N)ST was inactive with both the GoboH based acceptor (1.1%) and its deoxy analog (2.5%). The sialyltransferase from LNCaP utilized these two acceptors at lower activities of 34.3% and 9.6% respectively. Key results regarding the specificity for GloboH precursors were verified using mass spectroscopy in Table VI. Overall, these results demonstrate that 2-O-MeGalβ1,3GlcNAc is a unique acceptor for α2,3(N)ST. All enzymes can also be distinguished by their action on the Globo H precursor and its deoxy analog.

Specificity of Sialyltransferases for Core-2 Structure

The STs acted on the core-2 tetrasaccharide and 3-O-methyl analogs of this molecule in a manner that was largely expected based on our above studies with disaccharides (Table I, part B). α2,3(N)ST and α2,6(N)ST showed specificity towards the Galβ1,4GlcNAcβ side chain in the tetrasaccharide while the sialyltransferases from LNCaP preferred to act on the Galβ1,3GalNAcα moiety. While α2,3(N)ST was 24.1% active towards Galβ1,3GalNAcα-O—Al it displayed somewhat lower activity (6.1% activity) towards 3-O-MeGalβ1,4GlcNAcβ1,6(Galβ1,3)GalNAcα-O-Bn. Surprisingly, α2,3(O)ST displayed activity towards both methyl analogs of the tetrasaccharide suggesting that it can have multiple sites of action including, but not limited to, the 3-position of Gal in Galβ1,3GalNAcα. This finding is elaborated upon later in Results.

Defining Specific Acceptors to Distinguish Between Sialyltransferases from LNCaP and α2,3(O)ST

The sialyltransferase from LNCaP exhibited strict specificity for Galβ1,3GalNAcα while α2,3(O)ST displayed broader specificity. This is evident upon comparison of the two methylated tetrasaccharides, 3-O-MeGalβ1,4GlcNAcβ1,6(Galβ1,3)GalNAcα-O-Bn and Galβ1,4GlcNAcβ1,6(3-O-MeGalβ1,3)GalNAcα-O-Bn. While the cloned enzyme was 100.0% and 49.6% active respectively towards these acceptors, the LNCaP enzyme was 100.9% and 3.0% active respectively. The strict specificity of LNCaP enzyme towards Galβ1,3GalNAcα is also evident from its negligible activity towards Galβ1,3GlcNAcβ1,3Galβ-O-Me (1.4%) as compared to the cloned enzyme (37.2%). Further, these two enzymes differ in their action since, while the cloned enzyme utilized both Galβ1,3GalNAcα-O—Al and the T-hapten in the mucin core 2 structure as acceptors to the same extent (112.0% and 100.0%, respectively.), the LNCaP enzyme was only ⅓ efficient towards Galβ1,3GalNAcα-O—Al (34.4% active). The cloned enzyme and the LNCaP enzyme differed in using Galβ1,3GlcNAcr3-O—Al (42.1% and 3.7% active respectively). Finally, the addition of Gal to the β1,6-linked GlcNAc of mucin core 2 structure increased the activity of LNCaP enzyme (from 60.4% to 100.0%) in contrast to the behavior of the cloned enzyme (from 120.5% to 100.0%).

In order to define a simple specific acceptor for assaying cloned α2,3(O)ST, in presence of other α2,3(O)STs like those resembling LNCaP ST, we examined the action of both enzymes on a series of disaccharide analogs, namely, 3-O-MeGalβ1,3 GalNAcα-O-Bn; 4-O-MeGalβ1,3 GalNAcα-O-Bn; 4-FGalβ1,3 GalNAcα-O-Bn; Galβ1,3(6-OMe)GalNAcα-O-Bn and 3-O-MeGalβ1,3(6-O-Me)GalNAcα-O-Bn along with Galβ1,3GalNAcα-O-Bn (Table II). Mass spectroscopy analysis was performed with some of the enzymatic products as discussed in Table VI. These studies were conducted at two different acceptor concentrations. We observed that the cloned enzyme exhibited 70.4% and 55.9% activities towards 3-O-MeGalβ1,3GalNAcα-O-Bn and 3-O-MeGalβ1,3(6-O-Me)GalNAcα-O-Bn respectively as compared to Galβ1,3GalNAcα-O-Bn at 0.75 mM, which is the saturation point for the maximum activity. LNCaPα2,3(O)ST exhibited negligible activities towards these acceptors (2.6% and 1.1% respectively). Hence, 3-O-MeGalβ1,3GalNAcα-O-Bn and 3-O-MeGalβ1,3(6-O-Me)GalNAcα-O-Bn could be used as specific acceptors for the cloned α2,3(O)ST enzyme. In fact, the latter compound may be absolutely specific for this enzyme since α2,6sialyltransferase acting on α-GalNAc cannot act on this acceptor.

Biosynthesis of Mucin Core-2 Based Glycans

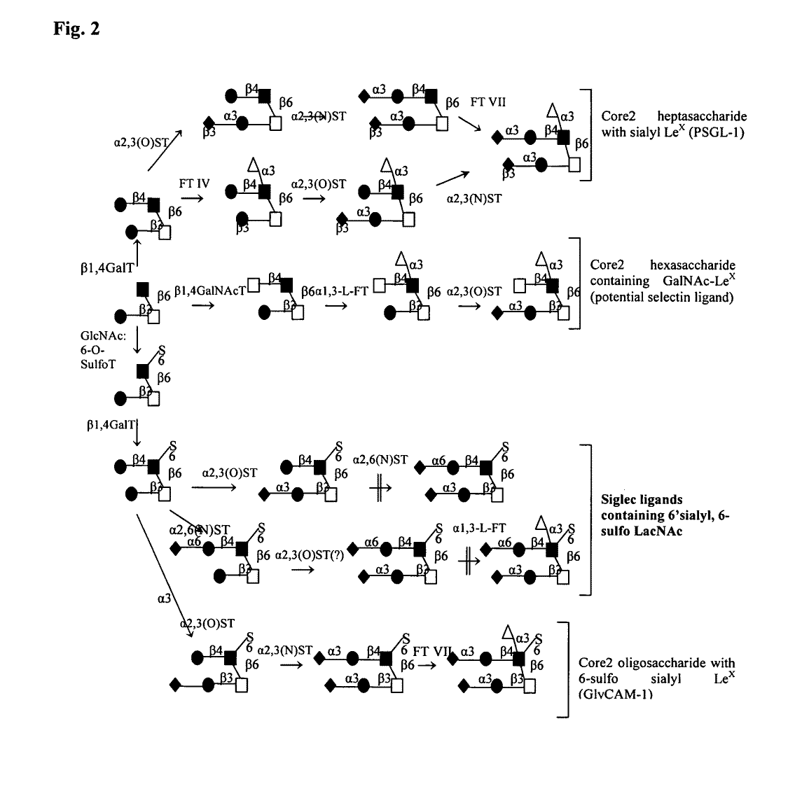

Enzyme specificity data of the present study (Table I, Part B) indicates the possible sequence in sialylation in the biosynthetic pathways of glycans such as PSGL-1, GalNAc Lewis-x, GlyCAM-1 and potential Siglec ligands (FIG. 2). However, one must be aware of the possibilities in vivo that a) the recombinant enzymes may not fully acquire the conformation of the native enzymes; b) the concentration of the substrates in the cell may dicate the action of enzymes in the competing pathways involved in multiple glycoconjugate synthesis; c) the conformation of the active site of these enzymes is sensitive to temperature and pH and d) alternative, albeit minor pathways for the same glycan elongation may exist in the cell.

A. PSGL-1 and GalNAc-Lewis x

Singly fucosylated mucin core-2 based glycans with terminal sialyl Lewis x structure and a tyrosine-sulfated peptide display high affinity binding for P- and L-selectin (32). Several investigator have also demonstrated that fucosylation can be the last step in the biosynthesis of glycans borne on PSGL-1 (33-37). Our enzymatic studies provide additional insight into the biosynthesis of such mucins.

First, our results suggest that α2,3 sialylation of Gal residue can proceed even after α1,3fucosylation of GlcNAc residue. In support of this proposition, we observed that Lewis x and (GalNAc) Lewis x determinants on the core-2 mucin did not affect the activity of clonal α2,3(O)ST. [Galβ1,4(Fucα1,3)GlcNAcβ1,6(Galβ1,3)GalNAcα-O-Me: 133.4% GalNAcβ1,4(Fucα1,3)GlcNAcβ1,6 (Galβ1,3)GalNAcα-O-Me: 134.9%]. Siayltransferase from LNCaP was also active towards the GalNAc Lewis-x containing mucin core 2 acceptor, albeit at a lower level (21.1% activity); Galβ1,4 (Fucα1,3)GlcNAcβ1,6(Galβ1,3)GalNAcα-O-Me and Galβ1,4(Fucα1,3)GlcNAcβ1,6(NeuAcα2,3Galβ1,3) GalNAcα-O-Bn served as acceptors for α2,3(N)ST to a significant, albeit low level (19.0% and 27.1% active respectively).

Second, regarding the sequence of sialylation, our results suggest that sialylation of β1,3-linked Gal must precede the sialylation of β1,4 linked Gal for favorable biosynthesis of mucin core 2 backbone compounds. This proposition is supported by the following lines of evidence: i) Sialylation of β1,3-linked Gal in mucin core 2 enhanced the α2,3 sialylation of β1,4 linked Gal via α2,3(N)ST as evident from a comparison of the activity of this enzyme towards Galβ1,4(Fucα1,3)GlcNAcβ1,6(Galβ1,3)GalNAcα- and Galβ1,4(Fucα1,3)GlcNAcβ1,6(NeuAcα2,3Galβ1,3)GalNAcα- (19.0% and 27.1%, respectively), and upon comparison of Galβ1,4(6-O-Sulfo)GlcNAcβ1,6(Galβ1,3)GalNAcα- with Galβ1,4(6-O-Sulfo)GlcNAcβ1,6(NeuAcα2,3Galβ1,3)GalNAcα- (28.1% and 50.8%, respectively; ii) When the LacNAc moiety of mucin core 2 was α2,3 sialylated, as evident from using the acceptor NeuAcα2,3Galβ1,4GlcNAcβ1,6(Galβ1,3)GalNAcα-O-Me, the α2,3 sialylation of Galβ1,3GalNAcα-moiety was greatly reduced (Cloned α2,3(O)ST enzyme 35.2% and LNCaP enzyme 5.1%).

Taken together, the above results suggest that Leukosialin would form from mucin core 2 tetrasaccharide by the sequential action of α2,3(O)ST and α2,3(N)ST. PSGL-1 glycans would form from further action of FTVII. PSGL-1 can also arise from mucin core 2 tetrasaccharide by the sequential action of FTIV (or possibly FTIII, FTV or FTVI) followed by α2,3(O)ST and α2,3(N)ST (FIG. 2). GalNAc-Lewis x type structures on the core-2 tetrasaccharide can likewise be formed by the sequential action of β1,4GalNActransferase, α1,3fucosyltransferase and α2,3(O)ST (FIG. 2).

B. GlyCAM-1

From previous studies, it is evident that GlcNAc:6-O-sulfotransferase does not act on the core-2 tetrasaccharide since it requires terminal GlcNAc for its action (38-40). This suggests that 6-sulfation precedes β1,4Galactosyltransferase activity in core-2 mucins. Building upon this observation, in the current work, we examined the action of sialyltransferases on an array of 6-O-sulfated structures to determine the potential biosynthetic pathway that leads to formation of sulfated core-2 structures. First, we observed that α2,3(N)ST acted more efficiently on the sialylated sulfated mucin core 2 compound Galβ1,4(6-O-Sulfo)GlcNAcβ1,6(NeuAcα2,3Galβ1,3)GalNAcα-O-Me (50.8% active) compared to Galβ1,4(6-O-Sulfo)GlcNAcβ1,6(Galβ1,3) GalNAcα-O-Me (28.1% active). Also, this 3′Sialyl,6-O-Sulfo Lewis x determinant dramatically decreased the activity of clonal α2,3(O)ST down to 7.4%. These observations support the proposition that, like PSGL-1, α2,3 sialylation of 6-O-SulfoLacNAc moiety in mucin core 2 is facilitated when the β1,3-linked Gal moiety has already been α2,3 sialylated. In other words, α2,3(O)ST action likely precedes α2,3(N)ST action in the case of GlyCAM-1 biosynthesis.

We observed that human FTVII enzyme, obtained from extracts of COS-7 cells that were transfected with FTVII cDNA, were capable of efficiently transferring [¹⁴C]Fuc to the acceptor NeuAcα2,3Galβ1,4(6-O-Sulfo)GlcNAcβ1,3Galβ1,4(6-O-Sulfo)GlcNAcβ-O-Me (Chandrasekaran, Xia, Neelamegham and Matta, unpublished results). Further the fucosylated acceptor Galβ1,4(6-O-Sulfo)(Fucα1,3)GlcNAcβ1,6 (NeuAcα2,3Galβ1,3)GalNAcα-O-Me had very low activity (1.7% active) towards α2,3(N)ST suggesting that fucosylation must occur after sialylation. Based on the above, we suggest that the biosynthesis of GlyCAM-1 starting from GlcNAcβ1,6(Galβ1,3)GalNAcα-, involves the sequential action of GlcNAc:6-O-Sulfo-T, β1,4Gal-T, α2,3(O)ST, α2,3(N)ST and finally FTVII (FIG. 2). The sialylation steps in this reaction scheme were validated by analyzing reaction products using mass spectroscopy (Table VI).

While our data above suggests that sulfation must be one of the first steps in the biosynthetic pathway, we also note that 6-O-Sulfation of β1,6-linked GlcNAc in mucin core 2 reduces the activity of α2,3(O)STs. In the regard, Galβ1,4(6-O-Sulfo)GlcNAcβ1,6(Galβ1,3)GalNAcα-O-Me was 27.2% active towards clonal α2,3(O)ST and 14.8% active towards sialyltranferase from LNCaP. Similarly, cloned α2,3(O)ST was 49.4% and 69.4% active with acceptors 6-O-SulfoGlcNAcβ1,6(Galβ1,3)GalNAcα-O-Bn and Galβ1,4(6-O-Sulfo)GlcNAcβ1,6(Galβ1,3)GalNAcα-O-Bn respectively. Not only 6-O-sulfation, but 3-O-sulfation of LacNAc moiety also decreased the enzyme activity of α2,3(O)ST to act on the core-2 mucin since 3-O-sulfo Galβ1,4 GlcNAcβ1,6 (Galβ1,3)GalNAcα-O-Bn was only 70.3% active. Our previous study indicated that sialylated core-2 acceptor, namely NeuAcα2,3Galβ1,3(GlcNAcβ1,6)GalNAcα-O-Bn, can act as an acceptor for GlcNAc:6-O-sulfotransferase (40). Thus, as an alternate scheme (not shown in FIG. 2), it may be possible that α2,3(O)ST action may precede 6-O-sulfation of GlcNAc in the GlyCAM-1 biosynthetic pathway.

C. Siglec Ligands

The present study reports the formation of α2,6sialylated and 6-O-sulfated mucin core-2 based compounds which may serve as potential ligands of Siglecs. In our experiments (Table I, part B), we observed that α2,6(N)ST was absolutely specific for the β1,4 linked Gal moiety in mucin core 2. Either α1,3 fucosylation or 6-O-Sulfation of the GlcNAc moiety reduced the activity β1.0% and 30.6%, respectively). Sialylation of β1,3-linked Gal in mucin core 2 in addition to 6-O-sulfation of GlcNAc moiety reduced the activity drastically: Galβ1,4(6-O-Sulfo)GlcNAcβ1,6(NeuAcα2,3Galβ1,3)GalNAcα-O-Me was active at 0.9% and Galβ1,4(6-O-Sulfo)(Fucα1,3)GlcNAcβ1,6 (NeuAcα-2,3Galβ1,3)GalNAcα-O-Me was 0% active. This suggests that action of α2,3(O)ST cannot be followed by α2,6(N)ST action in the case of 6-O-sulfated core-2 mucin (concept illustrated in FIG. 2). Galβ1,4(Fucα1,3)GlcNAcβ1,6(NeuAcα2,3Galβ1,3) GalNAcα-O-Bn, on the other hand, served as a reasonable acceptor for α2,6(N)ST β1.0%), suggesting that prior α2,3-sialylation of β1,3-linked Gal would not affect α2,6-sialylation of β1,4-linked Gal in non-sulfated mucin core 2 tetrasaccharide. Also, 3-O-sulfation of β1,3-linked Gal in mucin core 2 did not affect the activity of α2,6(N)ST (127.4%). Based on these results, the complex Siglec ligand, namely, NeuAcα2,6Galβ1,4(6-O-Sulfo)GlcNAcβ1,6(NeuAcα2,3Galβ1,3)GalNAcα- could arise from Galβ1,4(6-O-Sulfo)GlcNAcβ1,6 (Galβ1,3)GalNAcα- by the sequential action of α2,6(N)ST and α2,3(O)ST. The formation of a more complex ligand, namely, NeuAcα2,6Galβ1,4(Fucα1,3)(6-O-Sulfo)GlcNAcβ1,6(NeuAcα2,3Galβ1,3)GalNAcα- seems to be impossible since NeuAcα2,6Galβ1,4GlcNAc is inactive as an acceptor for α1,3-L-fucosyltransferases (41).

Mucin Core 2 Based Compounds as Acceptors for Sialyltransferases

We examined the action of the sialyltransferases on modified core-2 structures where key hydroxyl groups were replaced with either O-methyl or fluoro substituents (Table III). We observed that the cloned α2,3(O)ST utilized all acceptors including Galβ1,4GlcNAcβ1,6(3-FGalβ1,3)GalNAcα-O-Bn, Galβ1,4 GlcNAcβ1,6(3-O-MeGalβ1,3)GalNAcα-O-Bn and Galβ1,4 GlcNAcβ1,6(4-O-MeGalβ1,3)GalNAcα-O-Bn exhibiting 77.2%, 49.6% and 101.3% activity respectively. On the other hand, LNCaPα2,3(O)ST acted only on Galβ1,4GlcNAcβ1,6(4-O-MeGalβ1,3)GalNAcα-O-Bn (112.0%), and both 3-O-Methyl and 3-Fluoro substitution of the β1,3 linked Gal abrogated enzyme action. The cloned α2,6(N)ST showed very low activity towards 4-O-MeGalβ1,4GlcNAcβ1,6(Galβ1,3)GalNAcα-O-Bn (12.6%) whereas α2,3(N)ST exhibited 99.8% activity towards this acceptor. On the contrary, 3-O-Methyl, 3-Fluoro and 4-Fluoro substitution of Gal in the β1,4 linked Gal resulted in the formation of poor acceptors for both enzymes. The results indicate that besides transferring sialic acid to the 3-position of β1,3-linked Gal, α2,3(O)ST also induces sialylation of the acceptor at another position: either at the C-3 hydroxyl group of β1,4 linked Gal or at another —OH group on either β1,4 or β1,3 linked Gal.

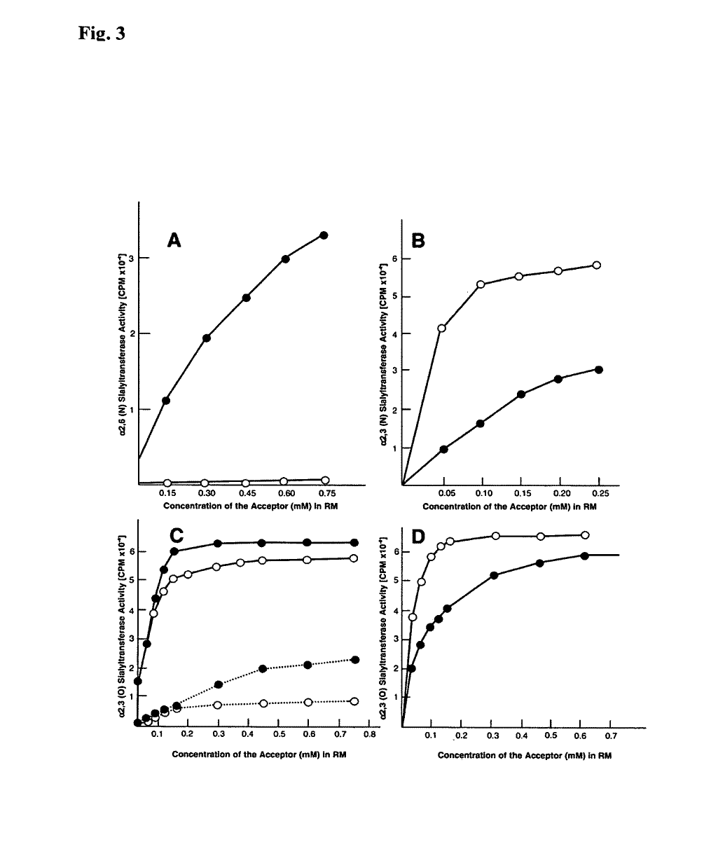

We examined in detail, the effect of 4-O-methylation of Gal moiety on the efficiency of mucin core 2 acceptors towards sialyltransferases (FIG. 3). When the activity of α2,6(N)ST was measured at varying concentrations of Galβ1,4GlcNAcβ1,6(Galβ1,3)GalNAcα-O-Bn and 4-O-MeGalβ1,4GlcNAcβ1,6(Galβ1,3) GalNAcα-O-Bn separately, it was found (FIG. 3A) that the latter compound was almost inactive at the maximum concentration (0.75 mM) tested whereas the former compound showed considerable activity even at the lowest concentration (0.15 mM) tested. When the activity of α2,3(N)ST was measured with the above two acceptors in the same manner (FIG. 3B), the latter compound was four fold active at the lowest concentration (0.05 mM) tested and two-fold active at the maximum concentration (0.25 mM) tested as compared to the activities of former compound at these concentrations. Thus 4-O-methylation of β1,4-linked Gal abolished the acceptor ability towards α2,6(N)ST but increased the acceptor efficiency considerably towards α2,3(N)ST.

When the activity of cloned α2,3(O)ST was measured at varying concentration of and Galβ1,4GlcNAcβ1,6(4-O-MeGalβ1,3)GalNAcα-O-Bn separately (FIG. 3C), the former compound was found as a slightly better acceptor at the standard incubation conditions. At a higher concentration of the donor CMP-NeuAc in the reaction mixture, the efficiency difference between these two acceptors was highly pronounced. On the contrary, LNCaPα2,3(O)ST (FIG. 3D) utilized the latter compound more efficiently as an acceptor, reaching the maximum activity at 0.15 mM whereas the former compound exhibited maximum activity at 0.60 mM. Thus 4-O-methylation of β1,3-linked Gal resulted in entirely different influence on the activities of cloned α2,3(O)ST and LNCaPα2,3(O)ST.

Probing the Location of Sialylation by Cloned α2,3(O)ST on the Acceptor GlcNAcβ1,6(3-O-MeGalβ1,3) GalNAcα-O-Bn

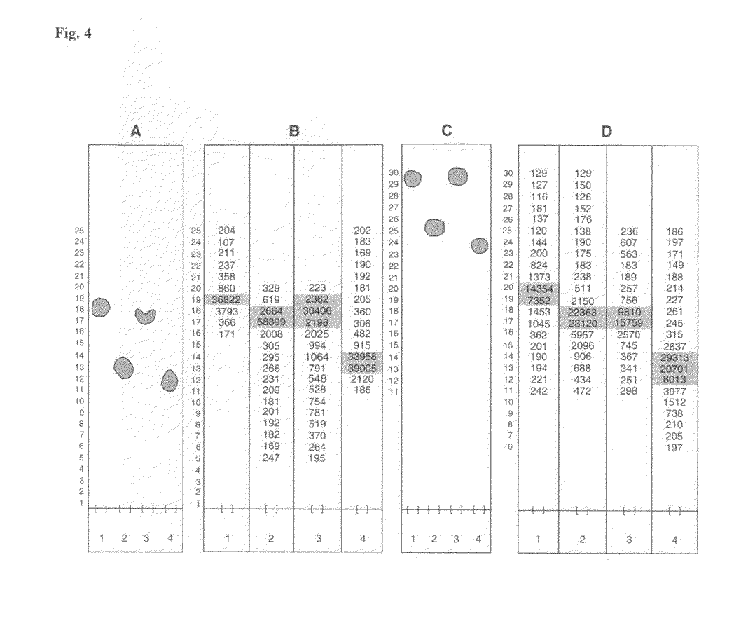

With the objective of determining if sialylation by cloned α2,3(O)ST takes place on the β1,4-linked Gal attached to GlcNAc, we examined the enzymatic products from the acceptors 3-O-MeGalβ1,3(GlcNAc β1,6) GalNAcα-O-Bn, Fucα1,2Galβ1,3 (GlcNAcβ1,6) GalNAcα-O-Bn and 3-O-Sulfo Galβ1,3(GlcNAcβ1,6)GalNAcα-O-Bn. The [9-³H] sialylated products from the acceptors 3-O-MeGalβ1,3(GlcNAcβ1,6)GalNAcα-O-Bn and Fucα1,2Galβ1,3(GlcNAcβ1,6)GalNAcα-O-Bn were isolated by Sep-Pak C18 method. The [9-³H] sialylated products from the acceptors, Galβ1,3(GlcNAcβ1,6)GalNAcα-O—Al and 3-O-SulfoGalβ1,3(GlcNAcβ1,6)GalNAcα-O-Bn were isolated by Biogel P₂ Column chromatography. These [9-³H] sialyl compounds were subjected to TLC using two different solvent systems (FIG. 4). The location of the radioactive compounds on TLC plates was determined by scraping ½ cm width segments followed by liquid scintillation counting. Such studies indicated that only a single product was being formed. It was found that the enzyme exhibited 24.7%, 45.4% and 54.7% activities respectively towards these acceptors when its activity towards reference molecule Galβ1,3(GlcNAcβ1,6)GalNAcα-O—Al was set to 100% (Table IV).

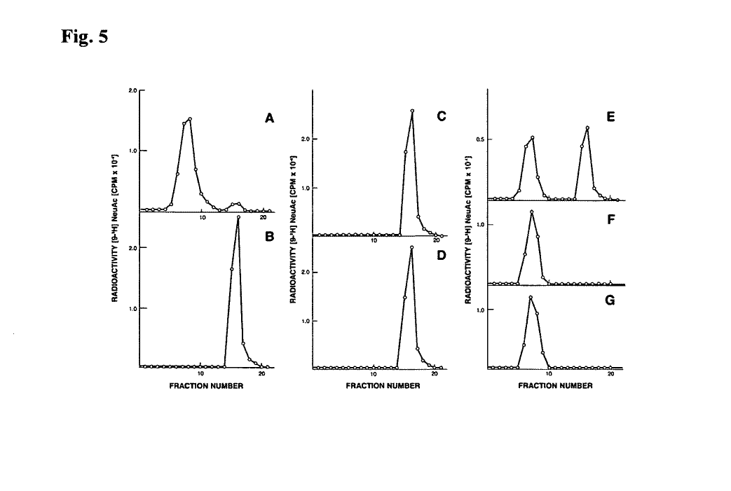

The sialylated products from all these acceptors except 3-O-SulfoGalβ1,3(GlcNAcβ1,6)GalNAcα-O-Bn exhibited binding to WGA-agarose via GlcNAc (FIGS. 5A, B, C and D). This indicates that 3-O-Sulfo group on β1,3-linked Gal in mucin core 2 trisaccharide abolishes the binding of WGA to β1,6 linked GlcNAc of mucin core 2. After digestion of these radioactive products with β-N-acetylhexosaminidase (Jack bean), the resulting radioactive compounds did not bind to WGA-agarose due to the absence of GlcNAc (FIGS. 5E, F and G). Overall, this set of experiments suggest that sialylation by α2,3(O)ST can take place on the β1,3-linked Gal at a position other than the 3-hydroxyl group.

Characterization of Sialylated Product Arising from Galβ1,4GlcNAcβ1,6(3-O-MeGalβ1,3)GalNAcα-O-Bn by the Action of Cloned α2,3(O)ST

We isolated the products by Sep-Pak C18 method and then subjected it to RCA-1-agarose chromatography (data not shown). As anticipated [9-³H]NeuAcα2,3Galβ1,4GlcNAcβ1,6(3-O-MeGalβ1,3) GalNAcα-O-Bn arising from the action of α2,3(N)ST did not bind to this column. The sialylated compounds formed from Galβ1,4GlcNAcβ1,6(3-O-MeGalβ1,3)GalNAcα-O-Bn and Galβ1,4GlcNAcβ1,6(Galβ1,3) GalNAcα-O-Bn by the action of cloned α2,3(O)ST, however, exhibited weak binding to this column. The results indicated very strongly that sialylation by α2,3(O)ST did not take place on the β1,4 linked Gal moiety, and this is in agreement with the data reported above using the WGA-agarose chromatography.

Evidence for Sialylation of Other than C-3 or C-6 Hydroxyl Group in the β1,3-Linked Gal Moiety

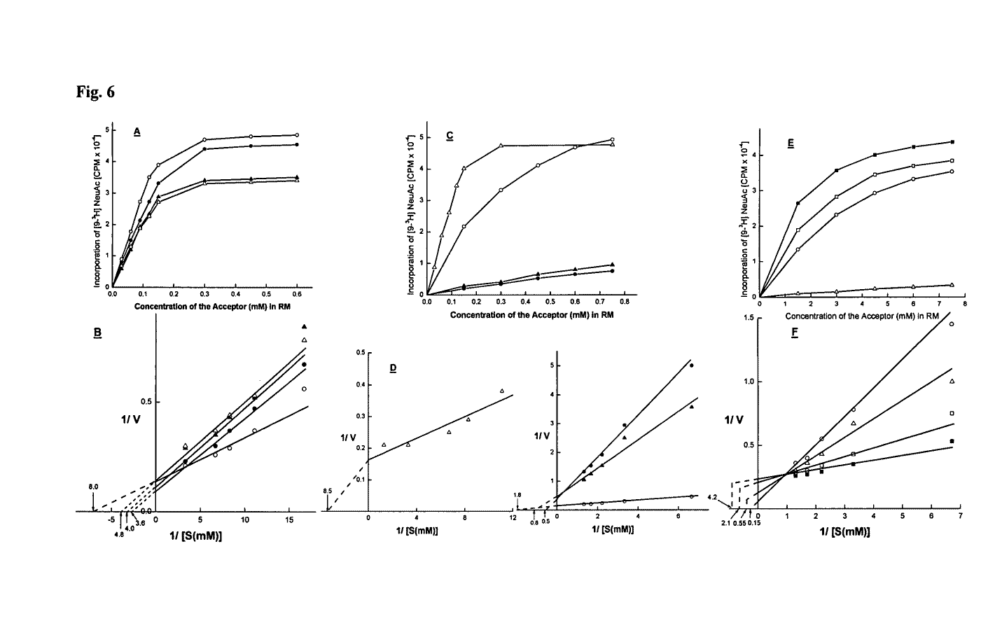

The action of α2,3(O)ST on a series of compounds over varying acceptor concentrations was examined in order to better define its specificity. In some of the molecules Gal was replaced by D-Fuc since both monosaccharides are identical, except that D-Fuc (6-deoxy Gal) lacks a hydroxyl group at the C-6 position. The compound D-Fucβ1,3GalNAcβ1,3Galα-O-Me as shown above served as good acceptor for the cloned α2,3(O)ST indicating that the C-6 OH group of β1,3-linked Gal may not be the target of sialylation. In order to gain evidence in support of this contention, we also examined D-Fucβ1,3GalNAcα-O-Bn; D-Fucβ1,3(GlcNAcβ1,6)GalNAcα-O-Bn; D-Fucβ1,3GalNAcβ1,3Galα-O-Me; Galβ1,3GalNAcβ1,3Galα-O-Me and 3-O—SulfoD-Fucβ1,3GalNAcβ1,3Galα-O-Me as acceptors for the cloned α2,3(O)ST (FIG. 6A and FIG. 6C). Both D-Fucβ1,3GalNAcα-O-Bn and D-Fucβ1,3(GlcNAcβ1,6)GalNAc—O-Bn at 0.75 mM concentration were found to be 100% efficient as acceptors as compared to 3-O-MeGalβ1,4GlcNAcβ1,6(Galβ1,3)GalNAcα-O-Bn. Further, [9-³H] sialyl compound arising from the former did not bind to WGA-agarose column whereas that from the latter did bind to this column (data not shown), indicating that sialylation occurs on the D-Fucosyl moiety only.

When the activity of the cloned α2,3(O)ST was measured by varying the concentration of Globo H analogs, we found (FIG. 6C) that Galβ1,3GalNAcβ1,3Galα-O-Me and D-Fucβ1,3GalNAcβ1,3Galα-O-Me were efficient acceptors, whereas both 3-O-SulfoGalβ1,3GalNAcβ1,3Galα-O-Me and 3-O-SulfoD-Fucβ1,3GalNAcβ1,3Galα-O-Me also served as acceptors but to a less extent. Detailed mass spectroscopy analysis was performed with 3-O-SulfoD-Fucβ1,3GalNAcβ1,3Galα-O-Me (compound S4 in Table VI) and its reaction product (compound 14) fragmentation analysis using MSⁿ analysis (Supplemental Data). This verified the attachment of sialic acid to this molecule, which is devoid of free hydroxyl at the C-3 and C-6 positions. D-Fucβ1,3GalNAcβ1,3Galα-O-Me was about 50% and 100% efficient as compared to Galβ1,3GalNAcβ1,3Galα-O-Me at 0.15 mM and 0.75 mM respectively, indicating that C-6 hydroxyl group is required for the maximum enzyme affinity towards the acceptors. Further, almost the same acceptor efficiency of 3-O-SulfoGalβ1,3GalNAcβ1,3Galα-O-Me and 3-O-SulfoD-Fucβ1,3GalNAcβ13,Galα-O-Me (FIG. 6C; Table V) would suggest that C-6 hydroxyl group of Gal moiety is unlikely the site of sialylation in the acceptor 3-O-SulfoGalβ1,3GalNAcβ1,3Galα-O-Me. Sialylation likely takes place at the C-2 or C-4 positions at β1,3 attached Gal.

The Unique Catalytic Activities of the Cloned α2,3(O)ST on the Modified Terminal β1,3Galactosyl Residues

A measurement of the clonal α2,3(O)ST activity at varying concentration of various acceptors followed by a determination of Km and Vmax values by Lineweaver-Burke plot (see FIG. 6 and Table V) indicated that the Globo backbone compounds Galβ1,3GalNAcβ1,3Galα-O-Me; 3-O-SulfoGalβ1,3GalNAcβ1,3Galα-O-Me; D-Fucβ1,3GalNAcβ1,3Galα-O-Me; the T-hapten compounds 4-O-MeGalβ1,3GalNAcα-O-Bn; 4-FGalβ1,3GalNAcα-O-Bn and D-Fucβ1,3GalNAcα-O-Bn and the mucin core 2 compounds D-Fucβ1,3(GlcNAcβ1,6)GalNAcα-O-Bn and Fucα1,2Galβ1,3(GlcNAcβ1,6)GalNAcα-O-Bn exhibited Km values (mM) in the range from 0.12 up to 2.00 and Vmax values (pmol/h) in the range from 197 to 1873. The results would indicate that the synthesis of these unique sialylated compounds is feasible by utilizing this enzyme.

ESI-MS/MSⁿ Analysis of the Enzymatically Sialylated Compounds

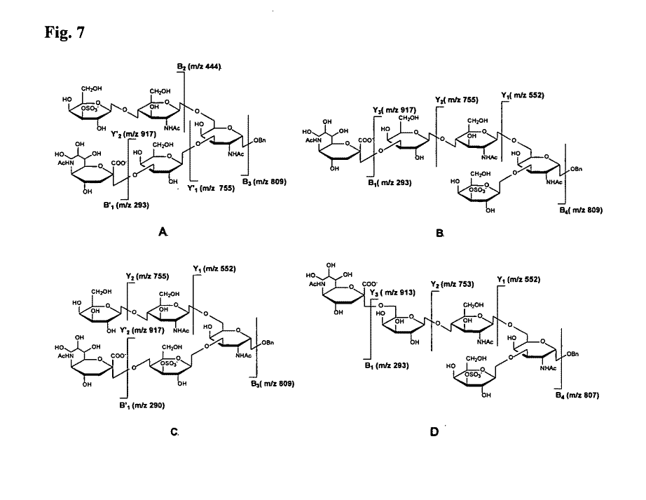

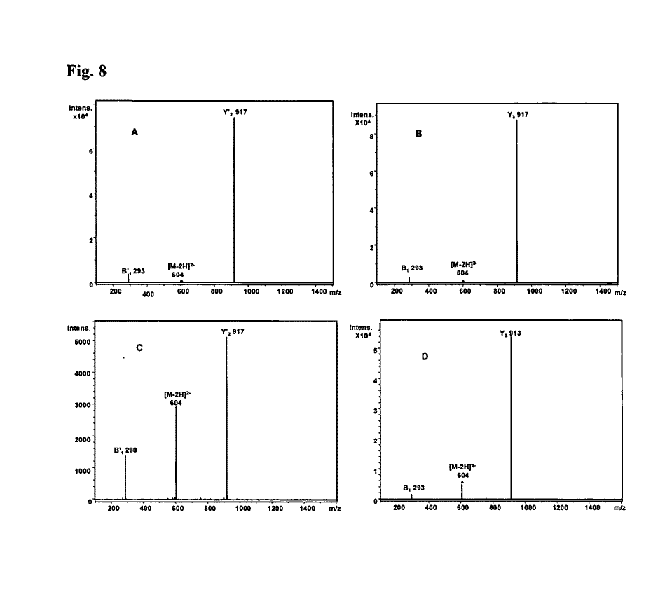

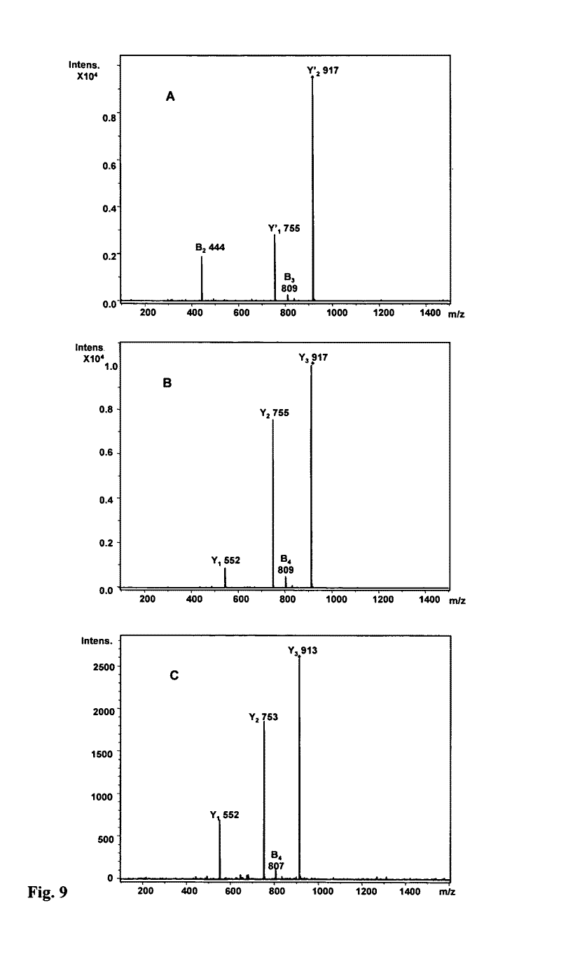

To confirm our finding that α2,3(O)ST can catalyze novel sialylation, we performed MSⁿ spectra analysis with selected enzymatically synthesized products under collision-induced dissociation (CID). Four isomeric pentasaccharides (compounds 6, 7, 18 and 23) with different sialyl linkages (siaα2,3, siaα2,2/4 and siaα2,6) and a sulfate residue (at 3-position of either galactose terminal in mucin core-2 tetrasaccharide) (FIG. 7) were detected as double charged ion [M-2H]²⁻ at m/z 604 in negative mode and doubly charged quadruply sodiated ions [M-2H+4Na]²⁺ at m/z 650 in positive mode (Table VI). MS² spectra of molecular species at m/z 604 derived from the four isomers are shown in FIG. 8. The fragmentations identified in FIGS. 8 and 9 were shown in FIG. 7 by using established Domon-Costello nomenclature (42). The glycosidic cleavage between sialyl group and branched mucin core-2 yielded B ions (B′₁ ion or B₁ ion) representing sialyl group and Y ions (Y′₂ ion or Y₃ ion) representing the sulfated branched mucin core-2 tetrasaccharides. As seen, the ratios of B ion to Y ions are significantly different among the different linkages (see FIG. 8). The same CID patterns are seen in FIGS. 8A and 8B indicating that compound 6 and 23 shared a common linkage, which is distinct from compound 7 and 18. Based on the enzymatic studies this is likely an α2,3 linkage. The Y₃ ion from compound 18 is at m/z 913 which is significantly different from the corresponding ions at m/z 917 from compound 6 and 23. This difference in molecular weight could be attributed to the formation of a lactone bond between two primary hydroxyl groups of Gal and GlcNAc in the Galβ1,4GlcNAc linkage of the branched mucin core 2 tetrasaccharide based on the mechanism reported by Ulrik et. al. (43) The spectrum of compound 7 (FIG. 8C) is distinct from the spectra of compounds 6, 18 and 23 indicating a different sialyl linkage and thus likely representing either α2,2 or α2,4 linkage of sialic acid.

MS³ spectra of the Y ions representing sulfated branched mucin core 2 structures are shown in FIG. 9. The B₂ ion at m/z 444 shown in FIG. 9A confirms that the sulfate group of 6 is on the β1,4 linked Gal of the mucin core-2 tetrasaccharide. Similarly, the Y₁ ions at m/z of 552 in FIGS. 9B and 9C indicate that the sulfate group of compound 23, 7 and 18 are on the Gal of Galβ1,3GalNAc in the tetrasaccharide. The formation of a lactone bond was also confirmed by the Y₂ ion at m/z 753 and B₄ ion at m/z 807 of compound 18 shown in FIG. 9C, which is different from the corresponding ion at m/z 755 and m/z 809 in FIGS. 9A and 9B.

The acceptor specificity of three cloned rat sialyltransferases and a human sialyltransferase from prostate cancer cell line LNCaP has been examined. By defining the specificity of these enzymes for an array of carbohydrate acceptors based on the core-2 and globo structures we: i) Define unique acceptors that can be used to characterize each of these enzymes in a complex mixture of glycosyltransferases, ii) Describe potential biochemical pathways that can lead to the synthesis of the ligands for selectins and Siglecs, and iii) Propose that the C-2 or C-4 position of β1,3 linked Gal in the core-2 mucin may be a site for the action of cloned α2,3(O)ST, provided the C-3 position has a substituent other than sialic acid.

Some Pertinent Observations on Rat Recombinant and LNCaP Sialyltransferases

Others investigators have performed studies with the sialyltransferases examined in the current manuscript. Chemo-enzymatic studies of Takano et. al. (44) identified only one product, namely Galβ1,4GlcNAcβ1,6(NeuAcα2,3Galβ1,3)GalNAcα-O—R, when the tetrasaccharide acceptor was incubated at 37° C. with the rat recombinant α2,3(O)ST. On the other hand, they identified the above monosialyl compound (8%) and disialyl compound (15%) in addition to the typical product, namely, NeuAcα2,3Galβ1,4GlcNAcβ1,6(Galβ1,3)GalNAcα-O—R (57%) when the tetrasaccharide acceptor was incubated at 37° C. with rat recombinant α2,3(N)ST at pH 7.4 for 24 h. Sengupta et. al. (45) made similar observation with the rat recombinant α2,3(N)ST when the tetrasaccharide acceptor was incubated at pH 7.4 at 37° C. for 18 h. In order to compare our findings with these investigators, we also extended the reaction times in our experiments from 2 h to 20 h under our standard experimental conditions (pH 6.0 at 37° C.) for the acceptor Galβ1,4GlcNAcβ1,6(Galβ1,3)GalNAcα-O-Bn and utilized all three rat recombinant enzymes α2,3(N)ST, α2,6(N)ST and α2,3(O)ST. The [9-³H] sialyl products arising from the tetrasaccharide acceptor were isolated using the Sep-Pak C18 cartridge. PNA-agarose affinity chromatography of the radioactive products showed that 92%, 93% and 0% of the radioactivity respectively were specifically bound to the column, indicating the identity of the bound products as [9-³H]NeuAcα2,3Galβ1,4GlcNAcβ1,6(Galβ1,3)GalNAcα-O-Bn and [9-³H] NeuAcα2,6Galβ1,4GlcNAcβ1,6(Galβ1,3)GalNAcα-O-Bn in case of α2,3(N)ST and α2,6(N)ST, and the unbound product as Galβ1,4GlcNAcβ1,6(9-³HNeuAcα2,3Galβ1,3)GalNAcα-O-Bn in case of α2,3(O)ST. Triton X-100 solubilized extract of LNCaP cells contained >95.0% α2,3(O)ST activity as evident from the activity of the following acceptors: 3-O-MeGalβ1,4GlcNAcβ1,6(Galβ1,3)GalNAcα —O-Bn (100.0%); Galβ1,4GlcNAcβ1,6(3-O-MeGalβ,13)GalNAc (3.0%) and Galβ1,4GlcNAcβ-O—Al (0.5%). Overall, our results are in agreement with other data in literature, and some differences observed may be attributed to variation in incubation conditions between the studies.

Unique Activities of Clonal α2,3(O)ST on a Different Position Other than C-3 of β1,3-Linked Gal

The clonal α2,3(O)ST was active with 3-O-MeGalβ1,4GlcNAcβ1,6(Galβ1,3)GalNAcα-O-Bn (100.0%) and Galβ1,4GlcNAcβ1,6(3-O-MeGalβ1,3)GalNAcα-O-Bn (49.6%) but was almost inactive towards Galβ1,4GlcNAcβ-O—Al (4.7%). When Galβ1,4GlcNAcβ1,6(Galβ1,3)GalNAcα-O-Bn was used as an acceptor, 100% of the isolated [9-³H] sialyl product did not bind to PNA-agarose column, indicating that sialylation took place only on the β1,3-linked Gal moiety. This suggests two possibilities. One, that sialylation might occur on β1,4-linked Gal when there is a C-3 block on β1,3-linked Gal moiety. Two, that sialylation may take place on some other position in β1,3-linked Gal moiety. In order to explore these possibilities, we used mucin core 2 trisaccharide containing different substituents on β1,3-linked Gal moiety as acceptor for the clonal α2,3(O)ST. Using tandem mass spectral analysis, WGA-agarose affinity chromatography on [9-³H] sialylated products before and after β-N-acetyl hexosaminidase treatment and also employing RCA-agarose affinity chromatography on [9-³H] sialyl products from mucin core 2 tetrasaccharide acceptors, we demonstrate unequivocally that sialylation took place on a different position of 3-O-substituted β1,3-linked Gal. Since both 3-O-Sulfo D-Fucβ1,3GalNAcβ1,3Galα-O-Me and 3-O-SulfoGalβ1,3GalNAcβ1,3Galα-O-Me served as acceptors with almost same efficiency for the clonal α2,3(O)ST, the C-6 position in C-3 blocked β1,3-linked Gal is unlikely to be the site for sialylation. It is possible that one of the remaining hydroxyl groups (C-2 or C-4) is the site of action of this enzyme. However, Kobata and his group (46) reported the occurrence of NeuAcα2,4Gal terminal N-glycan chains in Cold-insoluble globulin isolated from bovine plasma. Further, 3-SulfoGalβ1,3GalNAc β1,3Gal□1,4Gal□□Glc□ ceramide has been reported to occur in rat kidney (47). In fact, the sequence 3-Sulfo-Gal□13GalNAc□ is known to occur in several glycolipids (48).

Prediction of Potential Inhibitors of Siglec Binding

Our biochemical investigations suggest new molecules that may act as efficient inhibitors of Siglec binding to its ligand. In this regard, recently, Blixt et al. (49) assessed the sialoside specificity of the Siglec family using a novel multivalent platform comprising biotinylated sialosides bound to a streptavidin-alkaline phosphatase conjugate. Human Siglecs 2, 3, 7, 8, 9 and 10 exhibited significant binding to the sialoside-SAAP probe H containing NeuAcα2,6Galβ1,4GlcNAc. They observed that there was little correlation between the IC50 values and the ability of the corresponding SAAP probe to bind to immobilized Siglec chimeras. They also found in the binding assay that the length of the spacer arm can influence the interaction of a sialoside sequence with a given Siglec. In spite of these pitfalls, these authors came up with a remarkable finding that NeuAcα2,6(Galβ1,3)GalNAcα-O-Thr, NeuAcα2,3Galβ1,3GalNAcα-O-Thr and NeuAcα2,3Galβ1,3 (NeuAcα2,6)GalNAcα-O-Thr are 100, 600 and 3,000 fold more potent than NeuAcα2,6GalNAcα-O-Thr in inhibiting Siglec 4 (MAG) binding. Based on our studies, it now appears the following mucin core-2 based compounds may also be potential inhibitors for Siglec 4 and perhaps other Siglec binding also: NeuAcα2,3Galβ1,3(GlcNAcβ1,6)GalNAcα-; NeuAcα2,3Galβ1,3(NeuAcα2,3Galβ1,4GlcNAcβ1,6) GalNAcα-; NeuAcα2,3Galβ1,3(NeuAcα2,6Galβ1,4GlcNAcβ1,6) GalNAcα-; NeuAcα,α2,3Galβ1,3(6-O-Sulfo)GalNAcα-; NeuAcα2,3Galβ1,3(3-O-SulfoGalβ1,4GlcNAcβ1,6)GalNAcα-; NeuAcα2,3Galβ1,3[Galβ1,4(6-O-Sulfo)GlcNAcβ1,6]GalNAcα- and NeuAcα2,3Galβ1,3(6-O-SulfoGalβ1,4GlcNAcβ1,6) GalNAcα. In fact, blixt et. al. (50) have recently reported that NeuAcα2,6Galβ1,4(6-O-Sulfo)GlcNAcβ- is far better than the corresponding non-sulfated structure in binding to human CD22.

Enzymes that Act at the C-3 Position of Gal in LacNAc Also Act Efficiently on the Globo Acceptor

Earlier, we found that prostate cancer cell LNCaP α1,2-L-FT exhibited 4 fold greater activity towards β1,4-linked Gal as compared to its activity with β1,3-linked Gal in mucin core 2 tetrasaccharide (51); the same enzyme also utilized the Globo backbone structures Galβ1,3GalNAcβ13,Galα-O-Me and D-Fucβ1,3GalNAcβ1,3Galα-O-Me very efficiently. Subsequently, we observed that Gal:3-O-Sulfotransferase, Gal3ST-2, which exhibits specificity for LacNAc Type 2 structure also acts efficiently on the Globo structures (52). Extending these observations, in the present study, we show that α2,3(N)ST acted with equal efficiency on LacNAc and the Globo structure Galβ1,3GalNAcβ1,3Galα-O-Me. Thus, we discovered a physiological correlation that different enzymes modifying terminal β1,4-linked Gal also efficiently utilize the terminal β1,3-linked Gal in the Globo backbone.

Rationale for the Difference in the Acceptor Specificity of α2,3(O)ST from Liver and Prostate

All cloned mammalian sialyltransferases contain L- S- and VS-sialyl motifs (53-55). The L-sialyl motif was shown to bind to the donor CMP-NeuAc (56) while the S-motif participated in the binding of both donor and acceptor glycans (59). The exact role for VS-motif in the catalytic process had not been identified. Kitazume-Kawaguchi et al (60) suggested that His could be a catalytic residue for all sialyltransferases.

Three genes were identified to encode for human sialyltransferases ST-4a, b and c, which catalyze α2,3 sialylation of Galβ1,3GalNAcα-. Each enzyme followed a distinct pattern of tissue specific expression (59). The level of ST-4a mRNA was relatively similar in most tissues examined except for brain and skeletal muscle which show little and strong expression, respectively (59). ST-4-b was identified only in liver and, surprisingly, was not detectable in HepG₂; ST-4c was most strongly expressed in heart, placenta, testis and ovary. Prostate tissue also predominantly contain ST-4c (57). In the context of our study, it is possible that the human counterpart for rat liver recombinant α2,3(O)ST is ST-4-b, and based on the tissue of origin the sialyltransferases from prostate cells LNCaP may be ST-4c. It remains to be seen whether such a difference in the expression of ST-4 transcripts between liver and prostate can account for the difference in the intricate substrate specificities of enzymes from these sources.

Enzymatic Studies with Synthetic Acceptors can Complement Conventional Lectin and Antibody Studies

A differential expression of mRNAs for α2,3 and α2,6 sialyltransferases has been noted in rat and human tissues (60, 61). However, the expression of mRNA does not always correlate with actual enzyme levels, which in turn, cannot always predict glycosylation. Martin et. al. (62) examined the terminal glycans in tissues of normal mice and mice deficient in ST6Gal I or ST3Gal I, by using plant lectins as histochemical probes. They found cell type-specific expression of different linkages of terminal sialic acids in a variety of mouse tissues and identified multiple changes in cell type-specific glycan structures in various tissues of deficient mice. The present study was able to provide valuable information on the intricate substrate specificities of sialyltransferases. Such information will be helpful for meaningful interpretation of the histology data obtained using lectin and antibody probes on the tissues of normal and knockout mice. Studies of glycosyltransferase activity using an array of carbohydrate acceptors can also complement genomic and proteomic data that is becoming available from various high-throughput assays.

In order to test the hypothesis of predicting glycan signatures from the pattern of glycosyltrasferase activities, we first examined the levels of activities of various enzymes in the four cell lines ZR-75-1, T47D, MDA-MB-231 and MCF-7, followed by other cell lines as reported in Table II. The levels of the carbohydrate structures likely to arise from these enzyme activities are presented in Table III. Müller & Hanisch [2002] found that NeuAcα2,3Galβ1,3GalNAcα-units constituted the major structure of MFP6 proteins expressed in T47D (68.6%), MDA-MB-231 (65.5%) and ZR-75-1 (86.1%). The present invention also shows that α2,3-Sia-TI or TII was the most dominant enzyme as compared to α2,3-sia-TIV in all these cell lines. They also observed in MCF-7 that sialylated carbohydrate chains comprise only 5.2% of the O-glycan chains.

As found in accordance with the present invention, except for ZR-75-1, which exhibits high sialyltransferase activities, T47D, MDA-MB-231 and MCF-7 expresses almost the same level of α2,3-sia-TI or TII. MDA-MB231 and ZR75-1 cell extracts contain α2,3-Sia-T IV enzyme that constructs the NeuAc-2,3Galβ1,4GlcNAc and Müller & Hanisch [2002] report this sequence in the oligosaccharide chains of O-glycans of cell lines MDA-MB231 and ZR75-1 but not in MCF-7 cell line.

Enzymatic studies in accordance with the invention have demonstrated that MCF-7 does not contain α2,3-Sia-T IV but expresses α1,2-L-FT and α1,3-L-FT capable of generating the Fucα1,2Gal and Fucα1,3GlcNAc linkages, respectively. Thus, presence of these fucosyltransferases suggested the existence of Fucα1,2Galβ1,3GalNAc and Galβ1,4(Fucα,3)GlcNAc, the structures that were reported to be part of O-glycans in MCF-7 [Müller & Hanisch, 2002]. The lack of α2,3-sialyl-T IV activity suggests the absence of sialyl Lewis^{x} structure in MCF-7, which agrees with another report [Kumamoto et al. 1998]. However, Lewis^{x} structure moiety linked at C-6 position of GalNAc is reported to be part of O-glycans in MCF-7.

As a detailed example, The prostate carcinoma cell line, LNCaP, was grown in RPMI 1640 supplemented with 10% fetal bovine serum and the antibiotics (penicillin, streptomycin and amphotericin B) under conditions as recommended by American Type Culture Collection (Manassas, Va.). The cells were homogenized with 0.1M Tris-Maleate pH6.3 containing 2% Triton X-100 using a Dounce all glass hand-operated homogenizer. The homogenate was centrifuged at 16,000 g for 1 h at 4° C. Protein concentration in supernatant was measured using the BCA assay (Pierce Biotech, Inc., Rockford, Ill.) with BSA as the standard. The supernatant was adjusted to 5 mg protein/ml by adding the necessary amount of extraction buffer and then stored frozen at −20° C. until use. 10 μl aliquot of the extract was used in assays run in duplicate.

Rat recombinant αβ,3(O)ST, α2,3(N)ST and α2,6(N)ST were purchased from Calbiochem and stored at −20° C. or −70° C. as recommended by the supplier. Suitable aliquots were diluted with 1.0 ml of 0.1M NaCacodylate buffer pH6.0 containing 2% Triton CF-54 and 2% BSA and used in the experiments; α2,3(O)ST and α2,6(N)ST as diluted above were found to retain full activity for at least three months when stored frozen at −20° C. α.2,3(N)ST was diluted just before use in the experiment. 10 μl aliquots of the diluted enzymes were used in the assays run in duplicate.

The incubation mixtures run in duplicate contained 100 mM NaCacodylate buffer pH6.0, the acceptor (7.5 mM or as indicated in some experiments), CMP-[9-³H]NeuAc (0.2 μci; 20 μci/nmol or as indicated in some experiments) and the enzyme in a total volume of 20 μl. The control incubation mixtures contained everything except the acceptor. Incubation was carried out for 2 h at 37° C. The enzymatic transfer of [9-³H]NeuAc to a typical acceptor was linear for 2 h and less than 30% CMP-[9-3H]NeuAc was utilized. Chromatography, using either Dowex-1-Formate columns or Sep-Pak C18 cartridges was applied to separate radioactive product from unreacted CMP-[9³H]NeuAc as described below. The values for the duplicate runs did not vary by more than 5%. The radioactive products from neutral allyl and methyl glycosides as well as non-glycosides were measured by fractionation on Dowex-1-Formate (Bio-Rad: AG-1X8; 200-400 mesh; format form) as follows: The incubation mixture was diluted with 1.0 ml water and passed through AG-1-formate column (1.0 ml bed volume in a Pasteur pipette which had been washed with 5 ml of 2M Formic acid followed by 10 ml water). The column was washed twice with 1.0 ml water after the entry of the sample and then eluted with 3.0 ml of 0.1M NaCl. The radioactivity present in water and 0.1M NaCl eluates were measured separately using 3a70 scintillation cocktail (Research Products International, Mount Prospect, Ill.) and a Berkman LS6500 scintillation counter. The CPM values were corrected by subtracting the blank CPM. Any radioactivity present in the water eluate (as noticed in the case of some methylglycosides) was added to the corresponding CPM value of 0.1M NaCl eluate. The radioactive products from sulfated and/or sialylated methylglycosides were also measured by the above procedure, only the elution of AG-1-Formate column was continued further with 3.0 ml of 0.2M NaCl for achieving a complete elution of the radioactive product, a correction being made as before by subtracting the corresponding blank values.

The radioactive products from benzylglycosides and monosialylated benzylglycosides were measured by hydrophobic chromatography on Sep-Pak C18 cartridge (Waters, Milford, Mass.) and eluting the product with 3.0 ml methanol. The radioactivity was determined by liquid scintillation as above.

Reaction mixtures contained 0.3 μmol acceptor, 0.2 μmol CMP-NeuAc, 1.0 μCi CMP[9-³H]NeuAc, 30 μg BSA, 0.1M NaCacodylate pH 6.0 and 20 milliunit of the cloned sialyltransferase in a total volume of 100 μl and incubated for 18 h at 37° C. After incubation, the reaction mixture was diluted with 1.0 ml water and then subjected to column chromatography on a Biogel P₂ column (1.0×116.0 cm) using 0.1M pyridine acetate pH 5.4 eluting in the buffer. The first peak containing the radioactive product was collected, lyophilized to dryness, dissolved in 200 μl water and stored frozen at −20° C. until mass spectral analysis. MSⁿ experiments were carried out with an Esquire-LC, Bruker-HP ion trap (Bremen, Germany).

The target compounds are specifically designed to measure a particular sialyltransferase activity in presence of other sialyltransferase and glycosyltransferase activities and hence will be very useful in measuring these activities accurately in biological samples.

As a further extension of the present invention it has been found that structural studies, using specific substates of the invention, on carbohydrate chains released from cancer associated glycoprotein antigens reveal major changes at the outer ends of the oligosaccharide chains in cancer. In accordance with the present invention, various cancer cell lines have been examined for the levels of fucosyl-, β-galactosyl, β-N-acetylgalactosaminyl-, sialyl- and sulfotransferase activities which generate the outer ends of the oligosaccharide chains in glycans for identifying the signature glycan structures expressed by cancer cells. We have identified glycoslytransferases activities at the levels that would give rise to non-reducing carbohydrate ends of O-glycans as reported by others in breast cancer cell lines T47D, ZR75-1, MCF-7 and MDA-MB-231. Two distinct Gal: 3-O-sulfotransferases, one being specific for T-hapten Galβ1→3GalNAcα- and other exhibiting vast preference for the Galβ1→4GlcNAc terminal unit in O-glycans are expressed respectively by breast and colon cancer cell lines. We extended our study to ovarian cancer cells SW626 & PA-1 and hepatic cancer cells HepG₂. Our studies show that α1,2-L-fucosyl-T, α2,3(N) sialyl-T, and 3-O-Sulfo-T capable of acting on mucin core 2 tetrasaccharide Galβ1,4GlcNAcβ1,6(3-O-MeGalβ1,3)GalNAcα- can act on Globo H antigen backbone Galβ1,3GalNAcβ1,3Galα-. Thus, our study is able to identify signature carbohydrate moieties belonging to certain cancer-associated glycolipids. Briefly, the present study finds i) 3′-Sulfo-T-hapten as a measure of the tumorigenic potential and 3□-sialyl T-hapten & Lewis^{a} as prevalent structures in breast cancer cells; ii) 3′-Sulfo Lewis^{x}, 3-O-Sulfo Globo unit and 3-Fucosyl Chitobiose core as unique structures associated with colon cancer cells; iii) favorable synthesis of polylactosamine chain and T-hapten in ovarian cancer cells as they contain negligible sialyltransferase activities and iv) 6′-sialyl LacNAc unit and 3□-sialyl T-hapten as the prevalent structure in hepatic cancer cell glycans. Thus, it is evident from the present study that different cancer cells express unique glycan epitopes, thus providing novel targets for cancer diagnosis and treatment.

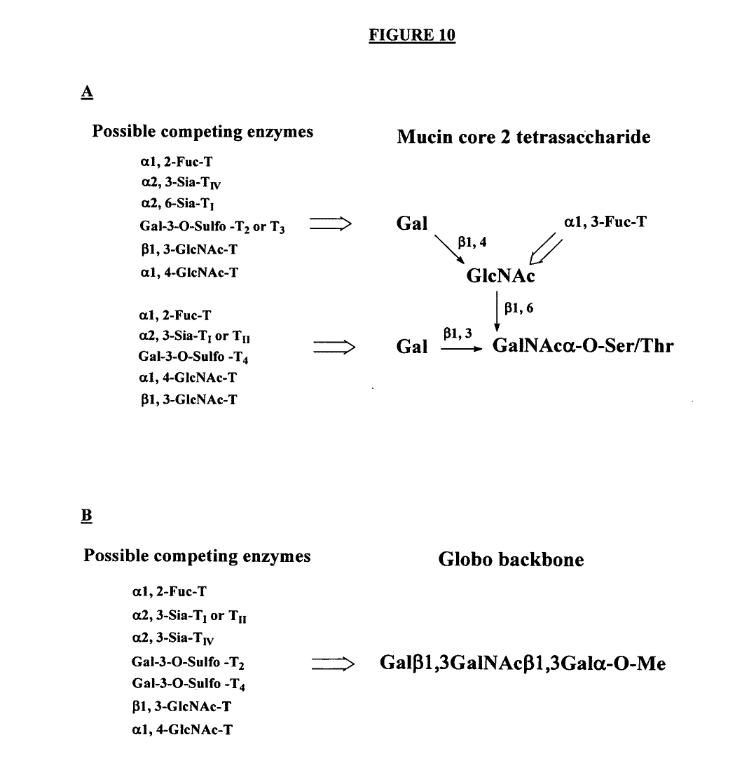

The structural variability of glycans is dictated by tissue-specific regulation of glycosyltransferase genes, the availability of suguar nucleotides and competition between enzymes for acceptor intermediates during glycan elongation. The studies of Müller & Hanisch on recombinant MUC 1 probe expressed in four breast cancer cell lines indicate that epigenetic parameters like the rate of Golgi passage and the topology of the protein substrate within the Golgi compartment are less important with respect to the final glycosylation profiles than the cellular repertoire of glycosyltransferases. Several glycosyltransferases and Gal3-O-Sulfotransferases are capable of elongating mucin core 2 tetrasaccharide as well as the Globo backbone unit. Such action of these enzymes could lead to a complexity of cancer associated terminal glycan structures, as illustrated in FIG. 10.

Müller & Hanisch [2002] expressed a MUC1 fusion protein MFP6 in the breast cancer cell lines ZR-75-1, MDA-MB-231, MCF-7 and T47D and studied the O-glycans of the fusion proteins after releasing by hydrazinolysis and then labeling with 2-amino benzamide. In order to test the hypothesis of predicting glycan signatures from the pattern of glycosyltrasferase activities, we first examined the levels of activities of various enzymes in these four cell lines followed by other cell lines as reported in Table VII. The levels of the carbohydrate structures likely to arise from these enzyme activities are presented in Table VIII. Müller & Hanisch [2002] found that NeuAcα2,3Galβ1,3GalNAcα-units constituted the major structure of MFP6 proteins expressed in T47D (68.6%), MDA-MB-231 (65.5%) and ZR-75-1 (86.1%). Our present study also showed that α2,3-Sia-TI or TII was the most dominant enzyme as compared to α2,3-sia-TIV in all these cell lines. They also observed in MCF-7 that sialylated carbohydrate chains comprise only 5.2% of the O-glycan chains. The present study finds that except for ZR-75-1, which exhibits high sialyltransferase activities, T47D, MDA-MB-231 and MCF-7 expressed almost the same level of α2,3-sia-TI or TH. MDA-MB231 and ZR75-1 cell extracts contain α2,3-Sia-T IV enzyme that constructs the NeuAc2,3Galβ1,4GlcNAc and Müller & Hanisch [2002] report this sequence in the oligosaccharide chains of O-glycans of cell lines MDA-MB231 and ZR75-1 but not in MCF-7 cell line. Our enzymatic studies have demonstrated that MCF-7 does not contain α2,3-Sia-T IV but expresses α1,2-L-FT and α1,3-L-FT capable of generating the Fucα1,2Gal and Fucα1,3GlcNAc linkages, respectively. Thus, presence of these fucosyltransferases suggested the existence of Fucα1,2Galβ1,3GalNAc and Galβ1,4(Fucα,3)GlcNAc, the structures that were reported to be part of O-glycans in MCF-7 [Müller & Hanisch, 2002]. The lack of α2,3-sialyl-T IV activity suggests the absence of sialyl Lewis^{x} structure in MCF-7, which agrees with another report [Kumamoto et al. 1998]. However, Lewis^{x} structure moiety linked at C-6 position of GalNAc is reported to be part of O-glycans in MCF-7. Overall the expression profiles of α(2,3)-sialyl-Ts and fucosyltransferases in our studies agree with the structures reported by Müller & Hanisch [2002] for these breast cancer cell lines.

Since sulfotransferases and sialyltransferases can compete for the same site of acceptors, it became important to study sulfotransferases. Among the breast cell lines, DU4475 is unique as it has negligible sialyltransferase activities but high level of 3-O-sulfotransferase specific for Galβ1,4GlcNAc. MCF-7 and ZR-75-1 also can express this enzyme at a low level. The presence of 1,3-FT would generate sulfo Lewis^{x} in these cell lines. MCF-7 contains both α1,2-L-FT and α1,3-L-FT activities which can generate Lewis^{y} moiety but it is not reported in O-glycans [Müller & Hanisch, 2002]. Gal3-O-Sulfo-T seems to prevent the action of α1,2-L-FT on Galβ1,4GlcNAc. The cell lines MDA-435/LCC6, MDA-435/LCC6^{MDR1} and even DU4475 have sulfotranferase activity which incorporates sulfate at C-3 position of galactose in T antigen to give 3′-Sulfo-T-hapten. We were the first to report that a core 2 structure distinguishes the sulfotransferase activities in breast and colon cell lines [Chandrasekaran et al., 1997; Chandrasekaran et al., 1999]. Two distinct types of Gal: 3-O-sulfotransferases were revealed, one being specific for the Galβ1,3GalNAcα-moiety is expressed by breast cancer cell lines and the other showing preference for the Galβ1,4GlcNAc branch in mucin core 2 is present in colon cancer cell lines and some breast cell lines [Chandrasekaran et al., 1999]. In this context, the availability of modified analogs of core 2 terasaccahride has proven to be important in determining the uniqueness of these enzymes. The specificity of sulfotransferase in colon cancer cell lines and DU4475 breast cell line predicts the presence of 3-O-Sulfo Galβ1,4GlcNAcβ1,6(Galβ1,3)GalNAcα-. On the other hand, Gal3Sulfo T-4 cloned by Seko et al. [2001] is present in breast cancer cell lines, synthesizing Galβ1,4GlcNAcβ1,6(3-O-SulfoGalβ1,3)GalNAcα sequence. These core 2 structures would serve as acceptors for α(2,3)-sialyltransferase and α(1,3)L-fucosyltransferase to generate more complex carbohydrate structures. The levels of glycosyltransferase and sulfotransferase activities in colon and as a comparison, that of ovarian cancer cell lines are reported in Table IX. A comparison of the levels of the predicted carbohydrate structures arising from these enzyme activities is presented in Table VIII. Three colon cell lines we have tested had very low α1,2-FT activity but a high level of both α1,3- and α,14-FT activities, the α1,4-FT activity being highest in Colo205. They all expressed FTVI activity, the level being highest in LS180. Colo205 showed a very weak expression of 3-O-Sulfotransferase activity towards Galβ1,4GlcNAcβ. On the contrary high levels of this enzyme activity are present in LS180 and SW1116. Since the levels of both α2,3-sia-TIV and α1,2-FT activities appear to be low, in all cases, this would suggest that Galβ1,4GlcNAcβ arm in core 2 tetrasaccharide is prone to the action of Gal.3-O-sulfotransferase to give 3-O-sulfoGalβ1,4GlcNAcβ. The dominancy of 3′Sulfo Lewis^{x} determinant in LS180 and SW1116 can be expected as these cells contain a high level of α1,3-L-fucosyltransferase activity. The enzyme α2,3-sia-TI or TII activity is dominated in LS180 and SW1116. The occurrence of 3′Sulfo Lewis^{x} determinant has also been demonstrated in mucin glycoprotein expressed by LS174T-HM7, a highly metastatic subline of colon carcinoma LS174T [Capon et al., 1997]. The expression of di-O-sulfated mucin core 2 tetrasaccharide seems to be favorable with DU4475 since it lacks all sialyltransferase activities. Our present findings demonstrate the power of well-defined acceptors for studying these enzymes. Brown et al. [2003] analyzed mRNA from LS180 cells using Glyco-V1 Genechip micorarrays and detected only α2,3-Sia-TIV. On the contrary, the present study identified α2,3-Sia-TI or TII as the overwhelming sialyltransferase activity not only in LS180, but also in other colon cell lines Colo205 and SW1116. In agreement with our present data on fucosyltransferase activities, they also detected FTIII and FTVI in LS180 cells. But the presence of FTVII in colon cancer cells could not be ruled out, when considering that Fetuin triant sialylglycopeptide acted as a good acceptor for the FTs of colon cancer cell lines. Glycosyltransferase expression in human colonic tissues was examined by Kemmner et al. [2003] using oligonucleotide microarrays. Their restricted analysis of 39 glycosyltransferases present on the Genechip U95A indicated that sialyltransferases α2,6-Sia-TI, α2,3-Sia-TIV, fucosyltransferases FTIII, FTVI and FTVIII (α1,6-L-FT) and also GalNAcT-1(polypeptide:GalNAc-T) and β1,4GalT-2 may be responsible for the aberrant biosynthesis of carbohydrates in colonic carcinogenesis and metastasis. Their findings seem to fit with the present data on the pattern of most of the glycosyltransferase activities obtained for colon cancer cell lines.

In accordance with the invention, we also investigated the specificity of α(2, 3) Sia-T, Gal-3-O-Sulfo-T and α1,2-Fuc-T using core 2 branched structure and Galβ1,3GalNAcβ1,3Galα-sequence which occurs in glycolipids as globo H precursor. The Globo H antigen was originally characterized by Hakomori and co-workers in a human breast cancer cell line [Bremer et al., 1984]. Immunostaining using murine MoAb MBr1 demonstrated this antigen in other human tissues including prostate cancer and small cell lung carcinoma. We reported that α1,2-Fuc-T in LNCaP cells acts more efficiently (4 fold) on the mucin core 2 Galβ1,4GlcNAcβ unit as well as on the Globo H precursor trisaccharide as compared to its activity towards Galβ1,3GalNAcβ-arm in mucin core-2 [Chandrasekaran et al., 2002]. In another study, we examined the specificity of three cloned Gal 3 sulfotransferases [Chandrasekaran et al., 2004]. Gal 3SulfoT-2 which is known to incorporate sulfate at C-3 position of galactose in Galβ1,4GlcNAcβ behaves similar to α1,2-Fuc-T transferase and α2,3-Sia-TIV towards these substrates; it acted with the same efficiency on both the Globo trisaccharide and core-2 tetrasaccharide. Gal-3-sulfoT4 can also utilize the globo H precursor to the same extent as Galβ1,3GalNAcα-. α2,3-Sia-TI or TII generates NeuAcα2,3Galβ1,3GalNAc— in mucin and also acts on Globo H precursor to give NeuAcα2,3Galβ1,3GalNAcβ1,3Galα→sequence. α2,3-sia-TII has been reported to be involved in the biosynthesis of NeuAcα2,3Galβ1,3GalNAcβ1,3Galα,3Galβ1,4GlcβCer [Saito et al., 2003; Maccioni et al., 1999]. There is some controversy on the specificity of α2,3-Sia-TIV [Kono et al., 1997]. Some groups report that this enzyme is involved in the synthesis of NeuAc2,3Galβ1,3GalNAcβ in glycolipids, while other groups suggest that it can generate NeuAc2,3Galβ1,4GlcNAcβ followed by fucosylation to give SLe^{x} [Kitagawa, 1994; Sasaki et al., 1993]. Our studies demonstrate clearly the specificity of α2,3-Sia-TIV towards mucin core 2 Galβ1,4GlcNAc unit as well as Galβ1,3GalNAcβ1,3Galα-sequence.

Our study indicates three types of sulfated carbohydrate epitopes 3-O-SulfoGalβ1,3GalNAcβ1,3Galα-, 3-O-SulfoGalβ1,3GalNAcα- and 3-O-SulfoGalβ1,4GlcNAcβ as the signatures of cancer cells. The 3-O-Sulfo-T activity towards Galβ1,4GlcNAcβ- in DU4475 is 18-fold that of MCF-7 and ZR-75-1, which are the only other two breast cancer cell lines expressing this activity. These results would indicate the signature carbohydrate structure expressed by DU4475 as 3□-Sulfo Lewis^{x}. It is interesting to note that the tumorigenic breast cell lines MDA-MB-435/LCC6 and MDA-MB-435/LCC6^{MDR1} exhibit about 9- and 4-fold Gal:3-O-Sulfotranferase activity specific for Galβ1,3 GalNAcα-unit as compared to its level in the non-tumorigenic parent cell line MDA-MB-435S. Further, the former cell lines contain 4-5-fold GlcNAc:β1,4Gal-T activity and less sialyltransferase activities as compared to the latter. This, it appears that an increase in Gal:3-O-Sulfotransferase with a concomitant decrease in Gal:3-O-Sialyltransferase acting on Galβ1,3GalNAcα- has a relationship to the tumorigenic potential of breast cancer cell lines. The absence of FT activities in MDA-MB-435 series would suggest that 3-O-SulfoGalβ1,3GalNAcα- as well as 3-O-SulfoGalβ1,4GlcNAcβ could be signature structures of tumorigenic breast cancer cells.

For comparison purposes, the enzyme activity levels of two ovarian cancer cell lines SW626 and PA-1 (Table IV) were examined and the predicted carbohydrate structures are presented in Table III. These cell lines contain only a small amount of sialyltransferase and glycan sulfotransferase activities and considerably a low level of α1,3-L-Fucosyltransferase activity as compared to the colon cancer cell lines, indicating that the biosynthesis of polylactosamine chain is quite favored in these cell lines. The hepatic cancer cell line HepG2 appears to be capable of synthesizing α2,6-sialylated N-glycans and α2,3-sialyl T-hapten. It is interesting to note that, except for the occurrence of a significant level of α1,6-L-FT activity, other FT activities are either negligible or absent in HepG2 thus indicating the existence of a reciprocal relationship in the expression of α2,6(N)Sialyltransferase and α1,2 and α1,3/4-L-Fucosyltransferases. From the level of α1,6-L-FT activity it is apparent that, as compared to T47D cell line, the other breast cell lines BT20, MCF-7 and MDA-MB-435 series, respectively, express about 2-, 3- and 4-fold and the rest, namely MDA-MB-231, DU4475 AND ZR-75-1 express about 5-fold α1,6-Fucosylated N-glycans. The colon cancer cells Colo205, LS180 and SW1116 contained almost the same level of α1,6-FT activity indicating the presence of same amount of α1,6-Fucosylated N-glycans. Both sialo and asialo Fetuin triantennary glycopeptides served as good acceptors for the fucosyltransferases of the colon cell lines, indicating the facile synthesis of sialyl Lewis^{x} and sialyl Lewis^{a}. Ancrod was also utilized as an acceptor that would further indicate the formation of sialyl Lewis^{a}. Hence, Colo205, LS180 and SW1116 could express sialyl Lewis^{a}, sialyl Lewis^{x} and sialyl T-hapten.

We compared a breast tumor with colon tumor for glycosyltransferase activities (see Table V). The colon tumor in comparison to the breast tumor had 18-fold α1,3-FT activity. The α1,4-FT activity in breast tumor was comparatively negligible; but there was a significant level of α1,2-FT activity. These FT activities, thus, mirrored the O-glycan chains from MUC1 fusion protein of MCF-7 reported by Muller and Hanisch. In contrast to the presence of Galβ1,4GlcNAc utilizing Gal:3-O-Sulfotransferase in MCF-7, ZR-75-1 and DU4475, the other Sulfo-T specific for Galβ1,3GalNAc was found in this breast tumor. We identified the latter enzyme in MDA-MB-435 series and DU4475 and in a number of breast tumor specimens in an earlier study [Chandrasekaran et al., 1999].

It is interesting to note that the tumorigenic cell lines MDA-435/LCC6 and MDA-435/LCC6^{MDR1} as compared to the non-tumorigenic parent cell line MDA-MB-435S express 4-5 fold GlcNAc:β1,3/4Gal-T activity; DU4475 expressing negligible amount of sialytransferase activities, as well as a low level of GlcNAcβ1,3/4Gal-T activity as compared to other cell lines, contains a high level of GlcNAc:β1,3/4GalNAc-T activity as well as α-GalNAc:β1,3Gal-T activity. In this context, it makes sense in comparing this data to the situation in breast tumor. As compared to the colon tumor specimen, the breast tumor specimen showed a high level of GlcNAc:β1,3/4GalNAc-T activity and a low level of α2,3(O)ST and α-GalNAc:β1,3Gal-T activities.

Our analysis of glycosyltransferase activities in a colon tumor tissue (see Table IV) indicated a pattern of fucosyltransferase activities akin to that of colon cancer cells being present in this tissue. From the levels of α1,2-FT activity in tumor specimens as measured with four different acceptors (see Table III), it became evident that Globo-based acceptors, namely Galβ1,3GalNAcβ1,3Galα-O-Me and D-Fucβ1,3GalNAcβ1,3Galα-O-Me are better acceptors than the Core 1 acceptor Galβ1,3GalNAcα-O—Al and Galβ-O-Bn for tumor α1,2-FT. Further, the dominancy of Gal:3-O-Sulfotransferase activity specific for Galβ1,4GlcNAc over α2,3 (N) sialyltransferase activity in colon cancer cell lines and colon tumor tissue would suggest that 3-Sulfo Lewis^{x} terminal carbohydrate would be a signature carbohydrate structure associated with colon cancer.

Further, it is quite interesting to note that while the breast tumor tissue akin to breast cancer cell lines was almost devoid of FTVI activity, the colon tumor tissue contained a significant level of this activity thus resembling the colon cancer cell lines. It appears that N-glycans with 3-Fucosyl Chitobiose core could be a signature carbohydrate structure associated with colon cancer.

The present invention has well documented that α2,3 (O) ST activity is the most predominant sialytransferase activity ranging 70-90% of the total sialyating activity in breast and colon cancer cell lines as well as in the two tumor tissues that were examined. We have also found (Chandrasekaran et al. submitted for publication) that cloned α2,3 (O) ST (ST3 Gal II) can sialylate very efficiently not only T-hapten (Galβ1,3GalNAcα-) and Globo backbone (Galβ1,3GalNAcβ1,3Galα-) but also LacNac type 1 (Galβ1,3GlcNAcβ-). We have shown previously [Chandrasekaran et al. 1997; Chandrasekaran et al.] that Gal:3-O-Sulfotransferases of human breast and colon tumor tissues, which act on Galβ1,3GalNAcα- and Galβ1,4GlcNAcβ- respectively, act poorly on Galβ1,3GlcNAcβ-. Thus, it appears that the formation of 3′-sialyl Lewis^{a} and 3′-sulfo Lewis x could be the favorable events in colon cancer. Further the predominancy of α2,3 (O) ST activity in breast and colon cancer cells precludes the possibility of mucin core 1 chain (Galβ1,3GalNAcα-O-Ser/Thr) elongation in these cells. But such as possibility seems to exist in ovarian cancer cells, as they express almost negligible amount of sialytransferase activities.

A knowledge of glycoconjugate biosynthesis and the existing structures of glycans is helpful in interpreting the glycosyltransferase activity patterns. For example, α2,6-Sia-TI prefers LacNAc type II glycan branches located at the α1-3 linked mannose branch of N-glycans [Joziasse et al., 1987]. A lack of core 2 β1,6GlcNAc-T activity can lead to O-glycans having extended core 1 [Ellies et al., 1998]. Le^{a}, Le^{b} and sialyl Le^{a} are known to be located at C-3 branch from GalNAcα. A monoclonal antibody specific for 3-O-sulfated Lewis a tetrasaccharide is capable of binding to sulfomucin, a high molecular weight glycoprotein in colon mucosa, suggesting the existence of 3-O-SulfoGalβ1,3(Fucα,4)GlcNAcβ1,3, Galβ1,3 as part of sulfomucin [Yamachika et al., 1997]. A decrease of this epitope in colon cancer is reported whereas the expression of sulfo Lewis^{x} is not altered [Yamachika et al., 1997]. Thus, in accordance with the invention, activities and specificity of the chain terminating enzymes such as fucosyl-, N-acetylgalactosaminyl, sialyl- and sulfotransferases from cancer cells and tissues along with a knowledge of oligosaccharide biosynthesis would help to determine and select the carbohydrate epitopes present at the outer ends of known as well as unidentified cancer-associated antigens for immunological targeting.

The colon carcinoma cell line, Colo205, the hepatic carcinoma cell line, HepG₂, the breast carcinoma cell lines, BT20 and MCF-7, and the ovarian teratocarcinoma cell line, PA-1, were grown in minimal essential medium; the colon carcinoma cell line, LS180, and the breast carcinoma cell line, DU4475, were grown in RPCI 1640; the colon carcinoma cell line, SW1116, the ovarian carcinoma cell line, SW626, the breast carcinoma cell lines, MDA-MB-231, MDA-MB-435S, MDA-435/LCC6 and MDA-435/LCC6^{MDR1} were grown in Leibovitz's L-15 medium. All media were supplemented with 10% fetal bovine serum and the antibiotics, penicillin, streptomycin and amphtoericin B in 250 mL T-flasks under conditions as recommended by American Type Culture Collection, except for DU4475, which was grown as a suspension. MDA-435/LCC6 and MDA-435/LCC6^{MDR1} were kindly provided by Dr. Ralph Bernacki of this Institute. The cells were homogenized with 0.1M Tris-Maleate pH 7.2 containing 2% Triton X-100 using a Dounce all glass hand-operated homogenizer. The homogenate was centrifuged at 16,000 g for 1 h at 4° C. Protein was measured on the supernatants by the BCA micro method (Pierce Chemical Co.) with BSA as the standard. The supernatants were adjusted to 5 mg protein per mL by adding the necessary amount of extraction buffer and then stored frozen at −20° C. until use [Chandrasekaran et al., 2003]. Aliquots (10 μL) of the extracts were used in assays run in duplicate.

Colon tumor tissue (CC9338) and breast tumor tissue (BC9400) were obtained during surgical procedures at Roswell Park Cancer Institute and stored frozen within 1 h at −70° C. The tissues were homogenized at 4° C. with 4 volumes of 0.1M Tris Maleate pH 72 0.1% NaN₃ using kinematica. After adjusting the concentration of Triton X-100 to 2%, these homogenates were mixed in the cold room for 1 h using Speci-Mix (Thermolyne) and then centrifuged at 20,000 g for 1 h at 4° C. The clear fat-free supernatant was stored frozen at −20° C. until use. Aliquots of 10 μL from this extract were used in assays run in duplicates. Glycosyltransferase activity in cell lysate was determined by mixing the lysates with acceptor and radiolabeled donor (monosaccharide) under the reaction conditions detailed below, followed by separation of un-reacted donor from the radioactive product using anionic or hydrophobic chromatography. In all cases, the radioactive content of isolated products was determined by using 3a70 scintillation cocktail (Research Products International, Mount Prospect, Ill.) and a Beckman LS6500 scintillation counter. Controls for each assay contained the reaction mixture with everything except the acceptor. Radioactivity of product was subtracted from that of control to obtain the results presented in the Tables. All assays were run in duplicate. Results from duplicate runs did not vary by more than 5%.

The acceptors used for measuring the glycosyltransferase and sulfotransferase activities are given in Table I. The following are the conditions for individual enzymatic assays. Reaction temperature in all cases was 37° C. α2,3- and α2,6 sialytransferase (ST) assay reactions proceeded for 2 h in a mixture containing 100 mM sodium cacodylate buffer (pH 6.0), 7.5 mM acceptor, CMP-[9-³H]NeuAc (typically 0.2 μCi) and 10 μl cell extract in a total volume of 20 μl [Chandrasekaran et al., 1995]. βGlcNAc:β1,4Gal-T and αGalNAc:β1,3Gal-T assay mixtures in duplicate contained 0.1M Hepes-NaOH pH 7.0, 7 mM ATP, 20 mM Mn acetate, 1 mM UDP-Gal, UDP [¹⁴C]Gal (0.05 μCi; 327mCi/mmol; Amersham), 0.5 mM acceptor (unless otherwise stated) and the enzyme in a total volume of 20 μL. It was incubated for 4 h [Chandrasekaran et al., 2001]. βGlcNAc:β1,4GalNAc-T assay mixtures in duplicate contained 0.1M Hepes-NaOH pH 7.0, 7 mM ATP, 20 mM Mn acetate. UDP [³H] GalNAc (0.20 μCi; 7.8 Ci/mmol: New England Nuclear Corp.) 7.5 mM acceptor (unless otherwise stated) and the enzyme in a total volume of 20 μL and incubated for 4 h [Chandrasekaran et al., 2001].

α1,2, α1,6-, α1,3- and α1,4-fucosyltransferase (FT) assay reactions were carried out for 2 h in a reaction mixture containing 50 mM Hepes buffer (pH 7.5), 5 mM MnCl₂, 7 mM ATP, 3 mM NaN₃, 3 mM synthetic acceptor or 40 μg of fetuin-based acceptor, 0.05 μCi GDP-[14C]Fuc (290 mCi/mmol) and 10 μl cell extract in a total volume of 20 μl [Chandrasekaran et al., 1996]. Sulfotransferase (Sulfo T) assay reactions took 2 h and required a mixture containing 100 mM Tris-maleate (pH 7.2), 5 mM Mg Acetate, 5 mM ATP, 10 mM NaF, 10 mM BAL, 7.5 mM acceptor, 0.5 μCi of [³⁵S]PAPS (specific activity 2.4 Ci/mmol) and 10 μl of cell extract in a total volume of 30 μl [Chandrasekaran et al., 2004].

Dowex-1-C1 or Sep-Pak C18 cartridges were used to isolate radiolabeled product from the reaction mixture. For GalT, GalNAc-T and FT assays, the incubation mixture was diluted with 1 ml water and passed through a 1 ml Dowex-1-C1 column [Chandrasekaran et al., 1996; Chandrasekaran et al., 2001]. The column was washed twice with 1 ml of water. The breakthrough and the water wash contained the [¹⁴C]-galactosylated or [¹⁴C]-fucosylated products formed with neutral acceptors. 3 ml of 0.1M NaCl was used to obtain [¹⁴C]-fucosylated products from sialylated acceptors after water elution. For sialytransferase assays, the radioactive products from benzylglycosides were separated by hydrophobic chromatography on Sep-Pak C18 cartridge (Waters, Milford, Mass.), and elution of the product was done with 3 ml methanol [Palcic et al., 1988]. For sulfotransferase assays, elution of the [³⁵S]-sulfated compound from Dowex-1-C1 column was achieved by 3 ml of 0.2M NaCl [Chandrasekaran et al., 2004].

### REFERENCES INCORPORATED BY REFERENCE AS BACKGROUND ART

1. Varki, A. (1992) Diversity in the sialic acids□Glycobiology 2, 25-40.
2. Kelm, S., and Schauer, R. (1997) Sialic acids in molecular and cellular interactions, Int. Rev. Cytol. 175, 137-240.
3. Varki, A (1997) Sialic acids as ligands in recognition phenomena, FASEB J. 11, 248-255.
4. Karlsson, K. A. (1998) Meaning and therapeutic potential of microbial recognition of host glycoconjugates, Mol. Microbiol. 29, 1-11.
5. Crocker, P. R., Clark, E. A., Filbin, M., Gordon, S., Jones, Y., Kehrl, J. H., Kelm, S., Le Dourain, N., Powell, L., Roder, J., Schnaar, R. L., Sgroi, D. C., Stamenkovic, K., Schauer, R., Schachner, M., Van den Berg, T. K., Van der Merwe, P. A., Watt, S. M., and Varki, A. (1998) Siglecs: a family of sialic-acid binding lectins, Glycobiology 8. v.
6. Powell, L. D., and Varki, A. (1995) I-type lectins, J. Biol. Chem. 270, 14243-14246.
7. Kelm, S., Schauer, R., and Crocker, P. R. (1996) The sialoadhesins—A family of sialic acid-dependent cellular recognition molecules within the immunoglobulin superfamily Glycoconjugate J. 13, 913-926.
8. Powell, L. D., Sgroi, D., Sjoberg, E. R., Stamenkovic, I., and Varki, A. (1993) Natural ligands of the b-cell adhesion molecule cd22-β carry N-linked oligosaccharides with α-2,6-linked sialic acids that are required for recognition, J. Biol. Chem. 268, 7019-7027.
9. Cornish, A. L., Freeman, S., Forbes, G., Ni, J., Zhang, M., Cepeda, M., Gentz, R., Augustus, M., Carter, K. C., and Crocker, P. R. (1998) Characterization of siglec-5, a novel glycoprotein expressed on myeloid cells related to CD33, Blood, 92, 2123-2132.
10. Angata, T., and Varki, A. (2000) Siglec-7: a sialic acid-binding lectin of the immunoglobulin superfamily, Glycobiology 10, 431-438.
11. Bagriacik, E. U., and Miller, K. S. (1999) Cell surface sialic acid and the regulation of immune cell interactions: the neuraminidase effect reconsidered, Glycobiology 9, 267-275.
12. Bevilacqua, M. P., and Nelson, R. N. (1993) Selectins, J. Clin. Invest. 91, 379-387.
13. Varki, A. (1994) Selectin ligands, Proc. Natl. Acad. Sci. USA 91, 7390-7397.
14. Lasky, L. A. (1995) Selectin-carbohydrate interactions and the initiation of the inflammatory response, Annu. Rev. Biochem. 64, 113-139.
15. Rosen, S. D., and Bertozzi, C. R. (1994) The selectins and their ligands, Curr. Opin. Cell Biol. 6, 663-673.
16. Phillips, M. L., Nudelman, E., Gaeta, F. C. A., Perez, M., Singhal, A. K., Hakomori, S. I., and Paulson, J. C. (1990) Elam-1 mediates cell-adhesion by recognition of a carbohydrate ligand, sialyl-lex, Science 250, 1130-1132.
17. Walz, G., Aruffo, A., Kolanus, W., Bevilacqua, M., and Seed, B. (1990) Recognition by elam-1 of the sialyl-Le^{x} determinant on myeloid and tumor-cells, Science, 250, 1132-1135.
18. Brandley, B. K., Swiedler, S. J., and Robbins, P. W. (1990) Carbohydrate ligands of the lec cell-adhesion molecules, Cell. 63, 861-863.
19. Koenig, A., Jain, R., Vig. R., Norgard-Sumnicht, K. E., Matta, K. L., and Varki, A. (1997) Selectin inhibition: Synthesis and evaluation of novel sialylated, sulfated and fucosylated oligosaccharides, including the major capping group of GlyCAM-1, Glycobiology 7, 79-93.
20. Jain, R. K., Piskorz, C. F., Huang, B-G., Locke, R. D., Han, H-L., Koenig, A., Varki, A., and Matta, K. L. (1998) Inhibition of L- and P-selectin by a rationally synthesized novel core 2-like branched structure containing GalNAc-Lewis(X) and Neu5Ac a2-3Galβ1-3GalNAc sequences, Glycobiology 8, 707-717.
21. Chandrasekaran, E. V., Jain, R. K., Larsen, R. D., Wlasichuk, K., and Matta, K. L. (1995) Selectin ligands and tumor-associated carbohydrate structures—specificities of α-2,3-sialyltransferases in the assembly of 3′-sialyl-6-sulfo/sialyl lewis-a and lewis-x, 3′-sialyl-6′-sulfo lewis-x, and 3′-sialyl-6-sialyl/sulfo blood-group t-hapten, Biochemistry 34, 2925-2936.
22. Jain, R. K., Piskorz, C. F., and Matta, K. L. (1993) A convenient synthesis of n-acetyllactosamine-linked oligosaccharides from phenyl 3,6,2′,3′,4′,6′-hexa-o-acetyl-2-deoxy-2-phthalimido-1-thio-beta-lactopyranoside, Carbohydr. Res. 243, 385-391.
23. Jain, R. K., and Matta, K. L. (1996) Chemical synthesis of a hexasaccharide comprising the Lewis(x) determinant linked β-(1→6) to a linear trimannosyl core and the precursor pentasaccharide lacking fucose, Carbohydr. Res. 282, 101-111.
24. Jain R. K., Piskorz, C. F., Chandrasekaran, E. V., and Matta, K. L. (1998) Synthesis of Gal-β-(1→4)-GlcNAc-β-(1→6)-[Gal-β-(1→3)]-GalNAc-α-OBn oligosaccharides bearing O-methyl or O-sulfo groups at C-3 of the Gal residue: specific acceptors for Gal: 3-O-sulfotransferases, Glycoconj. J. 15, 951-959.
25. Xia, J., Alderfer, J. L., Piskorz, C. F., Locke, R. D., and Matta, K. L. (2003) Synthesis of fluorine-containing core-2 tetrasaccharides, Synlett. 9, 1291-1294.
26. Horejsi, V., Smolek, P., and Kocurek, J. (1978) Studies on lectins 0.35. Water-soluble o-glycosyl polyacrylamide derivatives for specific precipitation of lectins J. Biochim Biophys. Acta 538, 293-298.
27. Chandrasekaran, E. V., Rhodes, J. M., Jain, R. K., and Matta, K. L. (1994) Lactose as affinity fluent and a synthetic sulfated copolymer as inhibitor, in conjunction with synthetic and natural acceptors, differentiate human-milk lewis-type and plasma-type alpha-1-fucosyltransferases, Biochem. Biophys. Res. Commun. 198, 350-358.
28. Chandrasekaran, E. V., Rhodes, J. M., Jain, R. K., Bernacki, R. J., and Matta, K. L. (1994) A biosynthetic control on structures serving as ligands for selectins—the precursor structures, 3-sialyl/sulfo Gal-β-1,3/4GlcNAc-β-OR, which are high-affinity substrates for α-1,3/4-1-fucosyl-transferases, exhibit the phenomenon of substrate-inhibition, Biochem. Biophys. Res. Commun. 201, 78-89.
29. Chandrasekaran, E. V., Jain, R. K., Rhodes, J. M., Srnka, C. A., Larsen, R. D., and Matta, K. L. (1995) Expression of blood-group Lewis-b determinant from Lewis-a—association of this novel α(1,2)-L-fucosylating activity with the Lewis type α(1,3/4)-1-fucosyl-transferase Biochemistry 34:4748-4756.
30. Palcic, M. M., Heerze, L. D., Pierce, M., and Hindsgaul, O. (1988) The use of hydrophobic synthetic glycosides as acceptors in glycosyltransferase assays, Glycoconj. J. 5, 49-63.
31. Xue, J., Song L., Khaja D. S., Locke, D. R., West, C. M., Laine, R. A., and Matta, K. L. (2004) Determination of linkage position and anomeric configuration in Hex-Fuc disaccharides using electrospray ionization tandem mass spectrometry, Rapid Commun. Mass Spectrom. 18, 1947-1955.
32. Leppanen, A., Penttila, L., Renkonen, O., McEver, R. P., and Cummings, R. D. (2002) Glycosulfopeptides with O-glycans containing sialylated and polyfucosylated polylactosamine bind with low affinity to P-selectin. J. Biol. Chem. 277, 39749-39759.
33. Wilkins, P. P., McEver, R. P., and Cummings, R. D. (1996) Structures of the O-glycans on P-selectin glycoprotein ligand-1 from HL-60 cells, J. Biol. Chem. 271, 18732-18742.
34. Niemela, R., Natunen, J., Majuri, M. L., Maaheimo, H., Helin, J., Lowe, J. B., Renkonen, O., and Renkonen, R. (1998) Complementary acceptor and site specificities of Fuc-TIV and Fuc-TVII allow effective biosynthesis of sialyl-TriLex and related polylactosamines present on glycoprotein counterreceptors of selectins, J. Biol. Chem. 273, 4021-4026.
35. Britten, C. J., van den Eignden, D. H., McDowell, W., Kelly, V. A., Witham, J. J., Edbrooke, M. R., Bird, M. I., de Vries, T., and Smithers, N. (1998) Acceptor specificity of the human leukocyte α3 fucosyltransferase: role of FucT-VII in the generation of selectin ligands, Glycobiology 8, 321-327.
36. Stroud, M. R., and Holmes, E. H. (1997) Fucosylation of complex glycosphingolipids by recombinant fucosyltransferase-VII, Biochem. Bioplys. Res. Commun. 238, 165-168.
37. Koeller, K. M., and Wong, C. H. (2000) Chemoenzymatic synthesis of sialyl-trimeric-Lewis x, Chem. Eur. J. 6, 1243-1251.
38. Degroote, S., Lo-Guidice, J. M., Strecker, G., Ducourouble, M. P., Roussel, P., and Lamblin G (1997) Characterization of an N-acetylglucosamine-6-O-sulfotransferase from human respiratory mucosa active on mucin carbohydrate chains, J. Biol. Chem. 272, 29493-501.
39. Spiro, R. G., Yashimoto, Y., and Bhoyroo, V. (1996) Characterization of a rat liver Golgi sulphotransferase responsible for the 6-O-sulphation of N-acetylglucosamine residues in (3-linkage to mannose: Role in assembly of sialyl-galactosyl-N-acetylglucosamine 6-sulphate sequence of N-linked oligosaccharides, Biochem. J. 319, 209-216.
40. Chandrasekaran, E. V., Jain, R. K., Rhodes, J. M., Chawda, R., Piskorz, C., and Matta, K. L. (1999) Characterization of distinct Gal: 3-O-sulfotransferase activities in human tumor epithelial cell lines and of calf lymph node GlcNAc: 6-O-sulfotransferase activity, Glycoconj. J. 16, 523-536.
41. Chandrasekaran, E. V., Jain, R. K., Larsen, R. D., Wlasichuk, K., DiCioccio, R. A., and Matta, K. L. (1996) Specificity analysis of three clonal and five non-clonal α1,3-L-fucosyltransferases with sulfated, sialylated, or fucosylated synthetic carbohydrates as acceptors in relation to the assembly of 3′-sialyl-6′-sulfo Lewis x (the L-selectin ligand) and related complex structures, Biochemistry 35, 8925-8933.
42. Domon, B., and Costello, C. E. (1988) A systematic nomenclature for carbohydrate fragmentations in FAB MS/MS spectra of glycoconjugates, Glycoconj. J. 5, 397-409.
43. Jurva, U., Wikstrom, H. V., Weidolf, L., and Bruins A. P. (2003) Comparison between electrochemistry/mass spectrometry and cytochrome P450 catalyzed oxidation reactions, Rapid Commun. mass Sp. 17, 800-810.
44. Takano, Y., Kojima, N., Nakahara, Y., Hojo, H., and Nakahara, Y. (2003) Solid-phase synthesis of core 2 O-linked glycopeptide and its enzymatic sialylation, Tetrahedron 59, 8415-8427.
45. Sengupta, P., Misa, A. K., Suzuki, M., Fukuda, M., and Hindsgual, O. (2003) Chemoenzymatic synthesis of sialylated oligosaccharides for their evaluation in a polysialyltransferase assay, Tetrahedron Lett. 44, 6037-6042.
46. Takasaki, S., Yamashita, K., Suzuki, K., Iwanaga, S., and Kobata, A. (1979) Sugar chains of cold-insoluble globulin-protein related to fibronectin, J. Biol. Chem. 254, 8548-8553.
47. Tadano-Aritomi, K., Okuda, M., Ishizuka I., Kubo, H., and Ireland P. (1994) A novel mono-sulfated pentaglycosylceramide with the isoglobo-series core structure in rat kidney, Carbohydr. Res. 265, 49-59.
48. Teneberg, S., Miller-Podraza, H., Lampert H. C., Evans Jr., Evans, D. G., Danielsson, D., and Karisson, K. A. (1997) Carbohydrate Binding Specificity of the Neutrophil-activating Protein of Helicobacter pylori, J. Biol. Chem. 272, 19067-19071.
49. Blixt, O., Collins, B. E., van den Nieuwenhof, I. M., Crocker, P. R., and Paulson, J. C. (2003) Sialoside specificity of the siglec family assessed using novel multivalent probes—Identification of potent inhibitors of myelin-associated glycoprotein, J. Biol. Chem. 278, 31007-31019.
50. Blixt, O., Head, S., Mondala, T., Scanlan, C., Huflejt, M E., Alvarez, R., Bryan, M C., Fazio, F., Calarese, D., Stevens, J., Razi, N., Stevens, D J., Skehel, J J., van Die I., Burton, D R., Wilson, I A., Cummings, R., Bovin, N., Wong, C-H., and Paulson, J C. (2004) Printed covalent glycan array for ligand profiling of diverse glycan binding proteins, Proc. Natl. Acad. Sci. USA. 101(49),17033-17038.
51. Chandrasekaran, E. V., Chawda, R., Locke, R. D., Piskorz, C. F., and Matta, K. L. (2002) Biosynthesis of the carbohydrate antigenic determinants, Globo H, blood group H, and Lewis b: a role for prostate cancer cell α1,2-L-fucosyltransferase, Glycobiology 12, 153-162.
52. Chandrasekaran, E. V., Lakhaman, S. L., Chawda, R., Piskorz, C. F., Neelamegham, S., and Matta, K L. (2004) Identification of physiologically relevant substrates for cloned Gal:3-O-sulfotransferases (Gal3STs)—Distinct high affinity of Gal3ST-2 and LS180 sulfotransferase for the Globo H backbone, Gal3ST-3 for N-glycan multiterminal Galβ1,4GlcNAc β units and 6-sulfoGalβ1,4GlcNAcβ, and Gal3ST-4 for the mucin core-2 trisaccharide, J. Biol. Chem. 279, 10032-10041.
53. Drickamer, K. (1993) A conserved disulfide bond in sialyltransferases, Glycobiology, 3, 2-3.
54. Kurosawa, N., Hamamoto. T., Lee, Y-C., Nakaoka, T., Kojima, N., and Tsuji, S. (1994) Molecular-cloning and expression of GalNAcα-2,6-sialyltransferase, J. Biol. Chem. 269, 1402-1409.
55. Geremia, R. A., Harduin-Lepers, A., and Delannoy, P. (1997) Identification of two novel conserved amino acid residues in eukaryotic sialyltransferases: Implications for their mechanism of action, Glycobiology 7, v-vii.
56. Datta, A. K., and Paulson, J. D. (1995) The sialyltransferase sialylmotif participates in binding the donor substrate CMP-NeuAc, J. Biol. Chem. 270, 1497-1500.
57. Datta, A. K. Shinha, A., and Paulson, J. C. (1998) Mutation of the sialyltransferase S-sialylmotif alters the kinetics of the donor and acceptor substrates, J. Biol. Chem., 273, 9608-9614.
58. Kitazume-Kawaguchi, S., Kabata, S., and Arita, M. (2001) Differential biosynthesis of polysialic or disialic acid structure by ST8Sia II and ST8Sia IV, J. Biol. Chem., 276, 15696-15703.
59. Chang, M-L., Eddy, R. L., Shows, T. B., and Lau, J. T. Y. (1995) 3 genes that encode human beta-galactoside α-2,3-sialyltransferases—structural-analysis and chromosomal mapping studies Glycobiology 5, 319-325.
60. Paulson, J. C., Weinstein, J., and Schauer, A. (1989) Tissue-specific expression of sialyltransferases, J. Biol. Chem. 264, 10931-10934.
61. Kitagawa, H., and Paulson, J. C. (1994) Differential expression of 5 sialyltransferase genes in human tissues, J. Biol. Chem. 269, 17872-17878.
62. Martin, L. T., Marth, J. D., Varki, A., and Varki, N. M. (2002) Genetically altered mice with different sialyltransferase deficiencies show tissue-specific alterations in sialylation and sialic acid 9-O-acetylation, J. Biol. Chem. 277, 32930-32938.
63. Bast, R. C. Jr., Urban, N., et al. (2002) Early detection of ovarian cancer: promise and reality. Cancer Treat. Res., 107, 61-97.
64. Bonn D. (2002) Predictive value of PSA in prostate cancer is doubtful. Lancet Oncol., 3, 130.
65. Brawer M K. (1999) Prostate-specific antigen: current status. C.A. Cancer J. Clin., 49, 264-281.
66. Bremer, E. B., Levery, S. B., et al. (1984) Characterization of a glycosphingolipid antigen defined by the monoclonal antibody MBr1 expressed in normal and neoplastic epithelial cells of human mammary gland. J. Biol Chem, 259, 14773-14777.
67. Brockhausen I. (1999) Pathways of O-glycan biosynthesis in cancer cells. Biochim. Biophys. Acta., 1473, 67-95.
68. Brown J R, Fuster M M, et al. (2003) Expression patterns of α2,3,-Sialytransferases and α1,3-Fucosyltransferases determine the mode of sialyl Lewis x inhibition by disaccharide decoys. J. Biol. Chem., 278, 23352-23359.
69. Capon C, Wieruszeski J-M, et al. (1997) Sulfated Lewis X determinants as a major structural motif in glycans from LS174T-HM7 human colon carcinoma mucin. J. Biol. Chem., 272, 31957-31968.
70. Chandrasekaran, E. V., Chawda, R., et al. (2002) Biosynthesis of the carbohydrate antigenic determinants, Globo H, blood group H, and Lewis b: A role for prostate cancer cell α1,2-L-fucosyltransferase. Glycobiology, 12, 153-162.
71. Chandrasekaran, E. V., Chawda, R., et al. (2003) The binding characteristics and utilization of Aleuria aurantia, Lens culinaris and few other lectins in the elucidation of fucosyltransferase activities resembling cloned FT VI and apparently unique to colon cancer cells. Carbohydr. Res., 338, 887-901.
72. Chandrasekaran, E. V., Chawda, R., et al. (2001) Human ovarian cancer, lymphoma spleen, and bovine milk GlcNAc:β1,4Gal/GalNAc transferases: two molecular species in ovarian tumor and induction of GalNAcβ1,4Glc synthesis by α-lactalbumin. Carbohydr. Res., 334, 105-118.
73. Chandrasekaran, E. V., Chawda, R., et al. (2001) Human lung adenocarcinoma α1, 3/4-L-fucosyltransferase displays two molecular forms, high substrate affinity for clustered sialyl LacNAc type 1 units as well as mucin core 2 sialyl LacNAc type 2 unit and novel α1, 2-L-fucosylating activity. Glycobiology, 11, 353-364.
74. Chandrasekaran, E. V., Jain, R. K., et al. (1995) Selectin-ligands and tumor associated carbohydrate structures: specificities of α2,3-sialtransferases in the assembly of 3′-sialyl, 6-sulfo/sialyl Lewis a and x, 3′-sialyl, 6′-sulfo Lewis x and 3′-sialyl, 6-sialyl/sulfo blood group T-hapten. Biochemistry, 34, 2925-2936.
75. Chandrasekaran, E. V., Jain, R. K., et al. (1996) Specificity analysis of three clonal and five non-clonal a 1,3-L-fucosyltransferases with sulfated, sialylated, or fucosylated synthetic carbohydrates as acceptors in relation to the assembly of 3′-sialyl-6′-sulfo Lewis x (the L-selectin ligand) and related complex structures. Biochemistry, 35, 8925-8933.
76. Chandrasekaran, E. V., Jain, R. K., et al. (1999) Characterization of distinct Gal:3-O-sulfotransferase activities in human tumor epithelial cell lines and of calf lymph node GlcNAc:6-O-sulfotransferase activity. Glycoconj. 1, 16, 523-536.
77. Chandrasekaran, E. V., Jain, R. K., et al. (1997) The enzymatic sulfation of glycoprotein carbohydrate units: blood group T-hapten specific and two other distinct Gal:3-O sulfotransferases as evident from specificities and kinetics and the influence of sulfate and fucose residues occurring in the carbohydrate chain and on C-3 sulfation of terminal Gal. Glycobiology, 7, 753-768.
78. Chandrasekaran, E. V., Lakhaman, S. S., et al. (2004) Identification of physiologically relevant substrates for cloned Gal:3-O-sulfotransferases (Gal3STs): distinct high affinity of Gal3ST-2 and LS180 sulfotransferase for the globo H backbone, Gal3ST-3 for N-glycan multiterminal Galβ1, 4GlcNAcβ-units and 6-sulfoGalβ1, 4GlcNAcβ-, and Gal3ST-4 for the mucin core-2 trisaccharide. J. Biol. Chem., 279, 10032-10041.
79. Chandrasekaran, E. V., Rhodes, J. M., et al. (1994) A biosynthetic control on structures serving as ligands for selectins: The precursor structures. 3-sialyl/sulfoGalβ1.3/4GlcNAcβ-O—R which are high affinity substrates for α1.3/4-L-fucosyltransferases exhibit the phenomenon of substrate inhibition. Biochem. Biophys. Res. Comm., 201, 78-89.
80. Chandrasekaran, E. V., Rhodes, J. M., et al. (1994) Lactose as affinity eluent and a synthetic sulfated copolymer as inhibitor in conjunction with synthetic and natural acceptors differentiate human milk Lewis-type and plasma-type α-L-fucosyltransferases. Biochem. Biophys. Res. Commun., 198, 350-358.
81. Chu, T. M. (1997) Prostate-specific antigen and early detection of prostate cancer. Tumour Biol., 18, 123-134.
82. Dennis, J. W., Granovsky, M., et al. (1999) Glycoprotein glycosylation and cancer progression. Biochim. Biophys. Acta., 1473, 21-34.
83. Ellies, L. G., Tsuboi, S., et al. (1998) Core 2 oligosaccharide biosynthesis distinguishes between selectin ligands essential for leukocyte homing and inflammation. Immunity, 9, 881-890.
84. Fukuda M. (1996) Possible roles of tumor-associated carbohydrate antigens. Cancer Res., 56, 2237-2244.
85. Hollingsworth, M. A., Swanson, B. J. (2004) Mucins in cancer: protection and control of the cell surface. Nature Rev. Cancer, 4, 45-60.
86. Jain, R. K., Piskorz, C. F., et al. (1998) Synthesis of Galβ1, 4GlcNAcβ1, 6(Galβ1,3)GalNAcα-O-Bn oligosaccharides bearing O-methyl or O-sulfo groups at C-3 of the Gal residue: specific acceptors for Gal:3-O sulfotransferases. Glycoconj. 1, 15, 951-959.
87. Jain, R. K., Piskorz C. F., et al. (1993) A convenient synthesis of N-acetyllactosamine-linked oligosaccharides from phenyl 3.6.2′, 3′, 4′,6′-hexa-O-acetyl-2 deoxy-2-phthalimido-1-thio-β-lactopyranoside. Carbohydr. Res., 243, 385-391.
88. Izawa, M., Kumamoto, K., et al. (2000) Expression of sialyl 6-sulfo Lewis x is inversely correlated with convential sialyl Lewis x expression in human colorectal cancer. Cancer Res., 60, 1410-1416.
89. Joziasse, D. H., Schiphorst, W. E. C. M., et al. (1987) Branch specificity of bovine colostrum CMP-sialic acid: Galβ1→4GlcNAc—R α2→6-Sialytransferas. J. Biol. Chem., 262, 2025-2033.
90. Kannagi, R. (2004) Molecular mechanism for cancer-associated induction of sialyl Lewis x and sialyl Lewis a expression—The Warburg effect revisited. Glycoconj. J., 20, 353-364.
91. Kemmner, W., Foefzaad, C., et al. (2003) Glycosyltransferase expression in human colonic tissue examined by oligonucleotide arrays. Biochim. Biophys. Acta., 1621, 272-279.
92. Kim, Y. J., Borsig, L., et al. (1998) P-selectin deficiency attenuates tumor growth and metastasis. Proc. Natl. Acad. Sci. U.S.A., 95, 9325-9330.
93. Kim, Y. J., Borsig, L., et al. (1999) Distinct selectin ligands on colon carcinoma mucins can mediate patholocigal interactions among platelets, leukocytes and endothelium. Am. J. Pathol., 155, 461-472.
94. Kitagawa, H., Paulson, J. C. (1994) Cloning of a novel a 2,3-sialytransferase that sialylates glycoprotein and glycolipid carbohydrate groups. J. Biol. Chem., 269, 1394-1401.
95. Koike, T., Kimura, N., et al. (2004) Hypoxia induces adhesion molecules on cancer cells: A missing link between Warburg effect and induction of selectin-ligand carbohydrates. Proc. Natl. Acad. Sci. U.S.A., 101, 8132-8137.
96. Komminoth, P., Roth, J., et al. (1991) Polysialic acid of the neural cell adhesion molecule distinguishes small cell lung carcinoma from carcinoids. Am. J. Pathol., 139, 297-304.
97. Kono, M., Ohyama, Y., et al. (1997) Mouse beta-galactoside alpha 2,3-sialytransferases: Comparison of in vivo substrate specificities and tissue specific expression. Glycobiology, 7, 469-479.
98. Kumamoto, K., Mitsuoka, C., et al. (1998) Specific detection of sialyl Lewis x determinant carried on the mucin. Biochem. Biophys. Res. Commun., 247, 514-517.
99. Li, F., Wilkins, P. P., e. al. (1996) Post-translational modifications of recombinant P-selectin glycoprotein ligand-1 required for binding to P- and E-selectin. J. Biol. Chem., 271, 3255-3264.
100. Lillemoe, K. D., Yeo, C. J., et al. (2000) Pancreatic cancer: state-of the art care. Cancer J. Clin., 50, 241-268.
101. Maccioni, H. J., Daniotti, J. L., et al. (1999) Organization of gangliioside synthesis in the Golgi apparatus. Biochim. Biophys. Acta., 1437, 101-118.
102. Magnani, J., Steplewski, Z., et al. (1983) Identification of the gastrointestinal and pancreatic cancer-associated antigen detected by monoclonal antibody 19-9 in the sera of patients as a mucin. Cancer Res. 43, 5489-5492.
103. Mangray, S., King, T. C., (1998) Molecular pathobiology of pancreatic adenocarcinoma. Front. Biosci., 3, 1148-1160.
104. Mannori, G., Crottet, P., et al. (1995) Differential colon cancer cell adhesion to E, -P-, and L-selectin: role of mucin type glocoproteins. Cancer Res. 55, 4425-4431.
105. Milford, W. A., Catto, J. W. F., et al. (2001) Prostate specific antigen: biology, biochemistry and available commercial assays. Ann. Clin. Biochem. 38, 633-651.
106. Müller, S., Hanisch, F-G. (2002) Recombinant MUC1 probe authentically reflects cell-specific O-glycosylation profiles of endogenous breast cancer mucin: high density and prevalent core 2-based glycoslylation. J. Biol. Chem. 277, 26103-26112.
107. Ohyama, C., Hosono, M., et al. (2004) Carbohydrate structure and differential binding of prostate specific antigen to Maackia amurenis lectin between prostate cancer and benign prostate hypertrophy. Glycobiology, 14, 671-679.
108. Okada, T., Sato, Y., et al. (2001) Structural characteristics of the N-glycans of two isoforms of prostate-specific antigens purified from human seminal fluid. Biochim. Biophys. Acta., 1525, 149-160.
109. Palcic, M. M., Heerze, L. D., et al. (1988) The use of hydrophobic synthetic glycosides as acceptors in glycosyltransferase assays. Glycoconj. 5, 49-63.
110. Peracaula, R., Royle, L., et al. (2003) Glycosylation of human pancreatic ribonuclease: differences between normal and tumor states. Glycobiology, 13, 227-244.
111. Peracaula, R., Tabares, G., et al. (2003) Altered glycosylation allows the distinction between prostate-specific antigen (PSA) from normal and tumor origins. Glycobiology, 13, 457-470.
112. Piller F, Le Deist F, et al. (1991) Altered O-glycan synthesis in lymphocytes from patients with Wiskott-Aldrich syndrome. J. Exp. Med., 173, 1501-1510.
113. Prakash, S., Robbins, P. W. (2000) Glycotyping of prostate specific antigen. Glycobiology, 10, 173-176.
114. Saito, S., Aoki, H., et al. (2003) Human 3-sialtransferase (ST3Gal II) is a stage-specific embryonic antigen-4 synthase. J. Biol. Chem., 278, 26474-26479.
115. Sasaki, K., Watanabe, E., et al. (1993) Expression cloning of a novel Galβ(1-3/1-4)GlcNAcα2,3-sialtransferase using lectin resistance selection. J. Biol. Chem., 268, 22782-22787.
116. Seko, A., Hara-Kuge, S., et al. (2001) Molecular cloning and characterization of a novel human galactose 3-O-sulfotransferase that transfers sulfate to Galβ1-3GalNAc residue in O-glycans. J. Bio.l Chem., 276, 25697-25704.
117. Seko, A., Nagata, K., et al. (2002) Down-regulation of Gal 3-O sulfotransferase-2 (Gal3ST-2) expression in human colonic non-mucinous adenocarcinoma. Jpn. J. Cancer Res., 93, 507-515.
118. Velcich, A., Yang, W. C., et al. (2002) Colorectal cancer in mice genetically deficient in the mucin Muc2. Science, 295, 1726-1729.
119. Wong, N. K., Easton, R. L., et al. (2003) Characterization of the oligosaccharides associated with the human ovarian tumor marker CA125. J. Bio.l Chem., 278:28619-28634.
120. Yamachika, T., Nakanishi, H., et al. (1997) Reciprocal control of colon-specific sulfomucin and sialosyl-Tn antigen expression in human colorectal neoplasia. Virchows Arch, 431, 25-30.

TABLE I
Acceptor-substrate specificity analysis of the cloned sialyltransferases and LNCaP cell α2,3(O)ST

| | Sialyltransferase activity (%) | | | |
|---|---|---|---|---|
| | Cloned | Cloned | Cloned | LNCaP |
| Acceptor (7.5 mM) | α2,3(N)ST | α2,6(N)ST | α2,3(O)ST | α2,3(O)ST |

PART A:

| | | | | |
|---|---|---|---|---|
| Galβ1,4GlcNAcβ-O—Al | 100.0^{a} | 100.0^{b} | 4.7 | 0.5 |
| Galβ1,3GalNAcα-O—Al | 24.1 | 0.2 | 112.0 | 34.4 |
| Galβ1,4GlcNAcβ1,3Galβ1,4GlcNAcβ-O-Bn | | | 8.7 | |
| Galβ1,3(GlcNAcβ1,6)GalNAcα-O—Al | | | 120.5¹ | 60.4 |
| Galβ1,3GlcNAcβ1,3Galβ-O-Me | | | 37.2 | 1.4 |
| 2-O-MeGalβ1,4GlcNAc | 60.0 | 90.1 | 0.2 | |
| 3-O-MeGalβ1,4GlcNAc | 1.4 | 8.7 | 3.6 | |
| Galβ1,3GlcNAcβ-O—Al | 123.1 | 3.0 | 42.1 | 3.7 |
| 2-O-MeGalβ1,3GlcNAc | 115.3 | 3.7 | 2.9 | |
| 2-O-MeGalβ1,3GlcNAcβ-O-Bn | | 0 | | |
| Galβ1,3GalNAcβ1,3Galα-O-Me | 106.8²⁰ | 1.1 | 120.8² | 34.3 |
| D-Fucβ1,3GalNAcβ1,3Galα-O-Me | 3.1 | 2.5 | 115.6³ | 9.6 |
| Fetuin triantennary asialo gp (100 μg) | 66.1 | | | |
| Galβ1,3GalNAcα-O—Al/AA-CP (100 μg) | 1.7 | | | |

PART B:

| | | | | |
|---|---|---|---|---|
| Galβ1,4GlcNAcβ1,6(Galβ1,3)GalNAcα-O-Bn | 100.7 | 93.3 | 94.4⁴ | |
| 3-O-MeGalβ1,4GlcNAcβ1,6(Galβ1,3)GalNAcα-O-Bn | 6.1 | 4.4 | 100.0^{c} | 100.0^{d} |
| Galβ1,4GlcNAcβ1,6(3-O-MeGalβ1,3)GalNAcα-O-Bn | 95.3 | 97.7 | 49.6 | 3.0 |
| Galβ1,4(Fucα1,3)GlcNAcβ1,6(Galβ1,3)GalNAcα-O-Me | 19.0 | 7.1 | 133.4 | |
| Galβ1,4(Fucα1,3)GlcNAcβ1,6(NeuAcα2,3Galβ1,3)GalNAcα-O-Bn | 27.1 | 31.0 | | |
| NeuAcα2,3Galβ1,4GlcNAcβ1,6(Galβ1,3)GalNAcα-O-Me | | | 35.2 | 5.1 |
| GalNAcβ1,4(Fucα1,3)GlcNAcβ1,6(Galβ1,3)GalNAcα-O-Me | | | 134.9 | 21.1 |
| 6-O-SulfoGlcNAcβ1,6(Galβ1,3)GalNAcα-O-Bn | | | 49.4^{e,5} | |
| Galβ1,4(6-O-Sulfo)GlcNAcβ1,6(Galβ1,3)GalNAcα-O-Bn | | | 69.4^{e} | |
| Galβ1,4(6-O-Sulfo)GlcNAcβ1,6(Galβ1,3)GalNAcα-O-Me | 28.1²¹ | 30.6¹⁷ | | |
| Galβ1,4(6-O-Sulfo)GlcNAcβ1,6(NeuAcα2,3Galβ1,3)GalNAcα-O-Me | 50.8²² | 0.9 | | |
| Galβ1,4(6-O-Sulfo)(Fucα1,3)GlcNAcβ1,6(NeuAcα2,3Galβ1,3)GalNAcα-O-Me | 1.7 | 0 | | |
| NeuAcα2,3Galβ1,4(6-O-Sulfo)(Fucα1,3)GlcNAcβ1,6(Galβ1,3) | | | 7.4 | |
| GalNAcα-O-Me | | | | |
| 3-O-SulfoGalβ1,4GlcNAcβ1,6(Galβ1,3)GalNAcα-O-Bn | | | 70.3^{e,6} | |
| Galβ1,4GlcNAcβ1,6(3-O-SulfoGalβ1,3)GalNAcα-O-Bn | | 127.4^{e,18} | 33.1^{e,7} | |

100% represents
^{a}29520 CPM;
^{b}43500 CPM;
^{c}40560 CPM and
^{d}30930 CPM
^{e}These values were obtained by quantitating the [¹⁴C] sialylated products from fractionation of the reaction mixture on a Biogel P2 column
^{1-7,17-18,20-22}Mass spectroscopy analysis of these reaction products was performed in Table VI and supplemental data

TABLE II
Unique sialylation by cloned α2,3(O)ST results in identification of specific
acceptors that can measure α2,3(O)ST activity in the presence of related enzymes

| | Sialyltransferase Activity (%) | | | |
|---|---|---|---|---|
| | Acceptor (0.75 mM) | | Acceptor (7.5 mM) | |
| | Cloned | LNCaP | Cloned | LNCaP |
| Acceptor | α2,3(O)ST | α2,3(O)ST | α2,3(O)ST | α2,3(O)ST |
| Galβ1,3GalNAcα-O-Bn | 100.0⁸ | 100.0 | 100.0⁸ | 100.0 |
| | (42550 CPM) | (30490 CPM) | (43600 CPM) | (30600 CPM) |
| 3-O-MeGalβ1,3GalNAcα-O-Bn | 70.4 | 2.6 | 101.8 | 15.5 |
| 4-O-MeGalβ1,3GalNAcα-O-Bn | 104.6⁹ | 141.5 | 105.0⁹ | 188.7 |
| 4-F-Galβ1,3GalNAcα-O-Bn | 104.6¹⁰ | 44.8 | 102.3¹⁰ | 105.1 |
| Galβ1,3(6-O-Me)GalNAcα-O-Bn | 107.6¹¹ | 119.2 | 107.3¹¹ | 189.4 |
| 3-O-MeGalβ1,3(6-O-Me)GalNAcα-O-Bn | 55.9 | 1.1 | ND | ND |

ND: Not determined
⁸⁻¹¹Mass spectroscopy analysis of these reaction products was performed in Table VI and supplemental data

TABLE III
Sialyltransferase Activities towards Mucin Core 2 based Compounds

| | Cloned Sialyltransferase Activities | | | |
|---|---|---|---|---|
| | α2,6(N)ST | α2,3(N) ST | α2,3(O) ST | LNCaPα2,3(O)ST |
| Mucin Core 2 Compound [7.5 mM] | % | % | % | % |
| 3-O-MeGalβ1,4GlcNAcβ1,6(Galβ1,3)GalNAcα-O-Bn | 6.4 | 4.5 | 100.0^{c} | 100.0^{d} |
| Galβ1,4GlcNAcβ1,6(3-O-MeGalβ1,3)GalNAcα-O-Bn | 100.0^{a} | 100.0^{b} | 49.6 | 3.0 |
| 4-O-MeGalβ1,4GlcNAcβ1,6(Galβ1,3)GalNAcα-O-Bn | 12.6 | 99.8 | 103.0 | 97.1 |
| Galβ1,4GlcNAcβ1,6(4-O-MeGalβ1,3)GalNAcα-O-Bn | 101.3 | 100.6 | 101.3 | 112.0 |
| 3-FGalβ1,4GlcNAcβ1,6(Galβ1,3)GalNAcα-O-Bn | 11.3 | 7.2 | 102.9¹² | 91.5 |
| Galβ1,4GlcNAcβ1,6(3-FGalβ1,3)GalNAcα-O-Bn | 77.7¹⁹ | 96.9²⁵ | 77.2 | 1.7 |
| 4-FGalβ1,4GlcNAcβ1,6(Galβ1,3)GalNAcα-O-Bn | 2.7 | 7.9 | 102.1 | ND |

ND: Not determined
100% represents
^{a}30440 CPM;
^{b}38140 CPM;
^{c}39200 CPM and
^{d}30100 CPM
^{12,19,25}Mass spectroscopy analysis of these reaction products was performed in Table VI and supplemental data

TABLE IV
Unique sialylation of mucin core 2
based compounds by Cloned α2,3(O)ST

| | Sialyltransferase |
|---|---|
| Compound [7.5 mM] | activity (%) |
| Galβ1,3(GlcNAcβ1,6)GalNAcα-O—Al^{a} | 100.0 (29860 CPM)¹ |
| 3-O-MeGalβ1,3(GlcNAcβ1,6) GalNAcα-O-Bn^{b} | 24.7 |
| Fucα1,2Galβ1,3(GlcNAcβ1,6)GalNAcα-O-Bn^{b} | 45.4 |
| 3-O-SulfoGalβ1,3(GlcNAcβ1,6) GalNAcα-O-Bn^{a} | 54.7 |

^{a}The [9-³H] sialylated product was quantitated by BioGel P₂ Column Chromatography
^{b}Sep-Pak C18 fractionation procedure
¹Mass spectroscopy analysis of this reaction product was performed in Table VI and supplemental data

TABLE V
Discerning the unique catalytic abilities of the cloned α2,3(O)ST
by utilizing an array of synthetic compounds containing modified
terminal β1,3-Galactosyl residue as acceptors.

| | | | ^{a}CMP-NeuAc | |
|---|---|---|---|---|
| | | | (150 μM) | |
| | | | Km | Vmax |
| Compounds | | | (mM) | (pmol/h) |
| a) | Globo backbone: | | | |
| | i) | Galβ1,3GalNAcβ1,3Galα-O-Me | 0.12 | 1293¹³ |
| | ii) | 3-O-SulfoGalβ1,3GalNAcβ1,3Galα-O-Me | 1.25 | 416 |
| | iii) | D-Fucβ1,3GalNAcβ1,3Galα-O-Me | 0.56 | 1873 |
| | iv) | 3-O-SulfoD-Fucβ1,3GalNAcβ1,3Galα- | 2.00¹⁴ | 624 |
| | | O-Me | | |
| b) | T-hapten: | | | |
| | i) | 4-O-MeGalβ1,3GalNAcα-O-Bn | 0.13 | 1250 |
| | ii) | 4-FGalβ1,3GalNAcα-O-Bn | 0.25 | 1442 |
| | iii) | D-Fucβ1,3GalNAcα-O-Bn | 0.21¹⁵ | 1250 |
| c) | Mucin Core 2: | | | |
| | i) | D-Fucβ1,3(GlcNAcβ1.6)GalNAcα-O-Bn | 0.28¹⁶ | 1562 |
| | ii) | Fucα1,2Galβ1,3(GlcNAcβ1,6)GalNAcα- | 1.82 | 197 |
| | | O-Bn | | |

^{a}The reaction mixtures (20 μl) contained 0.2 μCi CMP[9-³H]NeuAc in 150 μM CMP-NeuAc
¹³⁻¹⁶Mass spectroscopy analysis of these reaction products was performed in Table VI and supplemental data. S4 (Table VI) is the synthetic acceptors that yields product¹⁴.

TABLE VI
Mass spectral analysis of enzymatically sialylated acceptors

| | | | | Found m/z | |
|---|---|---|---|---|---|
| Compound^{a} | | Yield^{c} | Calcd. | Negative | Positive |
| No. | Oligosaccharides | (nmol) | MW | Mode | Mode |
| 1 | 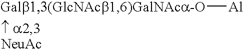 | 146 | 917.3 | [M − H]⁻: 916.0 | |
| 2 | 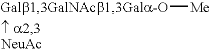 | 138 | 850.3 | [M − H]⁻: 848.9 | [M − H + 2Na]⁺: 894.7 |
| 3 | 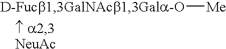 | 160 | 835.2 | [M − H]⁻: 833.8 | [M − H + 2Na]⁺: 879.2 |
| 4 | 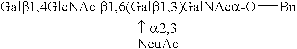 | 63 | 1129.4 | [M − H]⁻: 1127.1 | [M − H + 2Na]⁺: 1173.5 |
| 5 | 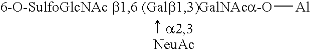 | 143 | 997.9 | [M − 2H]²⁻: 498.0 | [M − 2H + 4Na]²⁺: 544.0 |
| 6 | 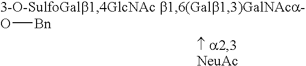 | 145 | 1210.1 | [M − 2H]²⁻: 604.1 | [M − 2H + 4Na]²⁺: 650.3 |
| 7 | 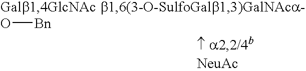 | 11 | 1210.1 | [M − 2H]²⁻: 604.1 | [M − 2H + 4Na]²⁺: 649.9 |
| 8 | 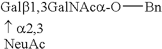 | 146 | 764.3 | [M − H]⁻: 763.4 | |
| 9 | 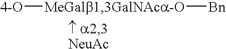 | 140 | 778.7 | [M − H]⁻: 777.6 | |
| 10 | 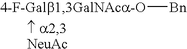 | 124 | 766.2 | [M − H]⁻: 764.9 | [M − H + 2Na]⁺: 813.4 |
| 11 | 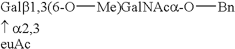 | 117 | 778.7 | [M − H]⁻: 777.5 | [M − H + 2Na]⁺: 823.3 |
| 12 | 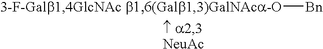 | 94 | 1131.4 | [M − H]⁻: 1129.4 | |
| 13 | 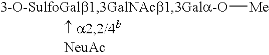 | 13 | 931.2 | [M − 2H]²⁻: 463.9 | |
| 14 | 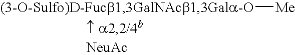 | 17 | 915.2 | [M − 2H]²⁻: 455.2 | |
| 15 | 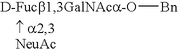 | 114 | 748.7 | [M − H]⁻: 747.6 | |
| 16 | 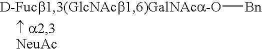 | 118 | 951.3 | [M − H]⁻: 949.7 | |
| 17 | 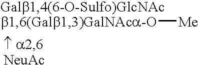 | 87 | 1134.0 | [M − 2H]²⁻: 566.8 | [M − 2H + 4Na]²⁺: 611.9 |
| 18 | 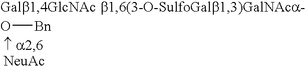 | 121 | 1210.1 | [M − 2H]²⁻: 604.3 | [M − 2H + 4Na]⁺: 649.6 |
| 19 | 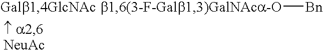 | 69 | 1131.4 | [M − H]⁻: 1128.9 | |
| 20 | 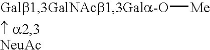 | 175 | 850.3 | [M − H]⁻: 849.0 | [M − H + 2Na]⁺: 895.0 |
| 21 | 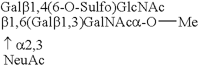 | 129 | 1134.0 | [M − 2H]²⁻: 567.2 | [M − 2H + 4Na]²⁺: 611.3 |
| 22 | 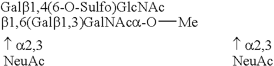 | 159 | 1423.0 | [M − 3H]³⁻: 472.2 | |
| 23 | 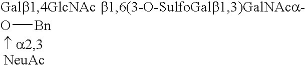 | 146 | 1210.1 | [M − 2H]²⁻: 603.4 | [M − 2H + 4Na]⁺: 649.5 |
| 24 | 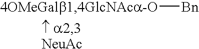 | 174 | 778.7 | [M − H]⁻: 777.3 | |
| 25 | 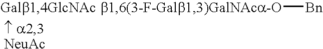 | 117 | 1131.4 | [M − H]⁻: 1130.3 | |
| S1 | 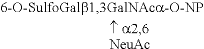 | | 875.8 | [M − 2H]²⁻: 437.1 | |
| S2 | 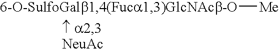 | | 914.3 | [M − 2H]²⁻: 456.1 | |
| S3 | 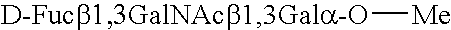 | | 543.0 | [M − H]⁻: 542.2 | [M − H + 2Na]⁺: 565.8 |
| S4 | 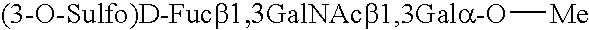 | | 623.0 | [M − H]⁻: 621.6 | [M − H + 2Na]⁺: 667.8 |

^{a}NeuAc denotes the sialic acid that was added to specific acceptors by the action of cloned sialyltransferases: α2,3(O)ST (Samples 1-16), α2,6(N)ST (Samples 17-19) and α2,3(N)ST (Samples 20-25). S1-S4 are chemically synthesized compounds
^{b}Sialylation may take place at either the C-2 or C-4 position of Gal in the Galβ1,3GalNac residue of the core-2 structure
^{c}Yield was calculated from the percentage of radioactivity incorporated into the acceptor from 200 nmol of CMP-[9-³H]NeuAc taken in the reaction mixture

TABLE VII
Acceptors used for assaying the enzymes in cancer cell lines

| Enzyme | Acceptor used for measuring the enzyme activity |
|---|---|
| Sialyltransferase | |
| α2,3-Sia-TI or TII | 3-O-MeGalβ1,4GlcNAcβ1,6(Galβ1,3)GalNAcα-O-Bn |
| (ST3:Galβ1,3GalNAc) | |
| α2,3-Sia-TIV | |
| (ST3:Galβ1,4GlcNAc) | Galβ1,4GlcNAcβ1,6(3-O-MeGalβ1,3)GalNAcα-O-Bn |
| α2,6-Sia-TI | |
| (ST6:Galβ1,4GlcNAc) | |
| α2,3-Sia-TIV | 2-O-MeGalβ1,3GlcNAcβ-O-Bn |
| Gal-/GalNAc-transferase | |
| β-GlcNAc:β1,4-Gal-T | 3-O-MeGalβ1,3(GlcNAcβ1,6)GalNAcα-O-Bn |
| β-GlcNAc:β1,4-GalNAc-T | 3-O-MeGalβ1,3(GlcNAcβ1,6)GalNAcα-O-Bn |
| α-GalNAc:β1,3-Gal-T | 4-F-GlcNAcβ1,6GalNAcα-O-Bn |
| Fucosyltransferase | |
| α1,2-Fuc-T | Galβ-O-Bn |
| α1,3-Fuc-T | 2-O-MeGalβ1,4GlcNAc |
| α1,4-Fuc-T | 2-O-MeGalβ1,3GlcNAc |
| | Fetuin triantennary asialo glycopeptide |
| | Ancrod |
| Fuc-TVI | GlcNAcβ1,4GlcNAcβ-O-Bn |
| Fuc-TVII | Fetuin triantennary sialo glycopeptide |
| α1,6-Fuc-T | Fetuin triantennary asialo agalacto glycopeptide |
| Sulfotransferase | |
| Gal3-O-Sulfo-T₂ or T₃ | Galβ1,4GlcNAcβ1,6(3-O-MeGalβ1,3)GalNAcα-O-Bn |
| Gal3-O-Sulfo-T₄ | 3-O-MeGalβ1,4GlcNAcβ1,6(Galβ1,3) GalNAcα-O-Bn |
| GlcNAc6-O-Sulfo-T | 3-O-MeGalβ1,3(GlcNAcβ1,6)GalNAcα-O-Bn |

TABLE VIII

| | MDA- | | | | |
|---|---|---|---|---|---|
| ENZYME | MB-231 | MCF-7 | T47D | ZR-75-1 | DU4475 |
| Sialyltransferases: | | | | | |
| α2,3(O)ST | 126.5 | 105.2 | 86.8 | 368.1 | 8.7 |
| α2,3(N)ST | 10.6 | 9.4 | 4.2 | 12.3 | 2.4 |
| α2,6(N)ST | 0 | 3.4 | 8.6 | 74.1 | 0 |
| Fucosyltransferases: | | | | | |
| α1,2-L-FT | 0.4 | 2.9 | 1.2 | 0.3 | 46.9 |
| α1,3-L-FT | 30.5 | 48.1 | 40.6 | 105.3 | 60.4 |
| α1,4-L-FT | 0.5 | 1.1 | 0.5 | 156.5 | 255.1 |
| FTV1 | 0 | 0.4 | 0.3 | 0 | 1.9 |
| α1,6-L-FT | 631.2 | 362.0 | 127.3 | 612.4 | 611.4 |
| FetTA-AsialoGP | 200.1 | 195.1 | ND | ND | 221.6 |
| FetTA-SialoGP | 0 | 0 | ND | ND | 31.7 |
| Ancrod | 0 | 0 | ND | ND | 33.8 |
| βGal/GalNAc-Transferases: | | | | | |
| GlcNAc:β1,4Gal-T | 494.4 (100.0) | 284.9 (100.0) | 358.1 | 552.7 | 155.5 (100.0) |
| GlcNAc:β1,4GalNAc-T | 152.1 (30.8) | 13.4 (4.7) | ND | ND | 103.3 (66.4) |
| αGalNAc:β1,3Gal-T | 28.8 | 23.3 | 30.6 | 36.3 | 47.4 |
| Glycan:Sulfotransferases | | | | | |
| Galβ1,3GalNAcα-: | | | | | |
| 3-O-Sulfo-T | 1.2 | 3.4 | 0.3 | 2.1 | 51.8 |
| Galβ1,4GlcNAcβ: | | | | | |
| 3-O-Sulfo-T | 0.2 | 19.4 | 0.3 | 19.1 | 361.2 |
| GlcNAc:6-O-Sulfo-T | 0.3 | 0 | 0 | 0.2 | 0.2 |

| | | MDA- | MDA- | MDA- |
|---|---|---|---|---|
| ENZYME | BT20 | MB435S | 435/LCC6 | 435/LCC6MDR1 |
| Sialyltransferases: | | | | |
| α2,3(O)ST | 197.8 | 257.4 | 171.6 | 159.1 |
| α2,3(N)ST | 18.3 | 26.3 | 18.4 | 17.7 |
| α2,6(N)ST | 0 | 86.5 | 56.0 | 64.1 |
| Fucosyltransferases: | | | | |
| α1,2-L-FT | 0.4 | 0 | 0 | 0.1 |
| α1,3-L-FT | 20.8 | 0 | 0 | 0 |
| α1,4-L-FT | 241.5 | 0 | 0 | 0 |
| FTV1 | 0 | 2.5 | 0 | 0 |
| α1,6-L-FT | 205.6 | 496.6 | 444.9 | 447.8 |
| FetTA-AsialoGP | 105.4 | 96.1 | 99.9 | 103.8 |
| FetTA-SialoGP | 9.6 | 0 | 0 | 0 |
| Ancrod | 41.3 | 0 | 0 | 0 |
| βGal/GalNAc-Transferases: | | | | |
| GlcNAc:β1,4Gal-T | 260.7 (100.0) | 80.1 (100.0) | 400.1 (100.0) | 295.8 (100.0) |
| GlcNAc:β1,4GalNAc-T | 33.1 (12.7) | 34.7 (43.3) | 86.2 (21.5) | 65.7 (22.2) |
| αGalNAc:β1,3Gal-T | 35.6 | 11.1 | 45.3 | 43.6 |
| Glycan:Sulfotransferases | | | | |
| Galβ1,3GalNAcα-: | | | | |
| 3-O-Sulfo-T | <0.1 | 7.2 | 62.6 | 28.1 |
| Galβ1,4GlcNAcβ: | | | | |
| 3-O-Sulfo-T | 0.3 | 0.1 | 0.5 | 0.3 |
| GlcNAc:6-O-Sulfo-T | 0.1 | 0 | 0.2 | 0.2 |

Sialyl-, Fucosyl-, β-Galactosyl-, β-N-Acetylgalactosaminyl- and Glycan:3-O-Sulfo-Transferase activities in breast cancer cell lines.
Activity: Incorporation of radioactive sugar or [35ₛ] sulfate [CPM × 10⁻³] catalyzed by 1 mg protein of the cell extract
ND: Not Determined

TABLE IX
A comparison of the levels of each potential signature carbohydrate structure among several cancer cell lines.

| | Breast Cancer Cell Lines | | | | | | | |
|---|---|---|---|---|---|---|---|---|
| | MDA-MB- | | | | | | MDA-MB- | MDA-435/ |
| Carbohydrate Structural Unit | 231 | MCF-7 | T47D | ZR-75-1 | DU4675 | BT20 | 435S | LCC6 |
| 6-Fucosyl Chitobiose core | 5X | 3X | X | 5X | 5X | 2X | 4X | 4X |
| ii) 3-Fucosyl Chitobiose core | O | T | T | O | T | O | T | O |
| iii) Lewis a | O | O | O | 6X | 10X | 10X | O | O |
| iv) Lewis x | X^{a} | 2X | 2X | 4X | 3X | X | O | O |
| v) 3′-Sialyl T-hapten | 4X | 3X | 3X | 12X | T | 6X | 9X | 6X |
| vi) 3′-Sulfo Lewis x | O | X | O | X | 18X | O | O | O |
| vii) 3′-Sialyl Lewis x | T^{b} | T | T | T | O | T | O | O |
| viii) 3′-Sialyl Lewis a | O | O | O | T | T | T | O | O |
| ix) T-hapten | T | T | T | T | T | T | T | T |
| x) 3′-Sulfo T-hapten | O | O | O | O | 7X | O | X | 9X |
| xi) 3′-Sialyl LacNAc | O | O | O | O | O | O | X | X |
| xii) 6′-Sialyl LacNAc | O | O | O | X | O | O | X | X |
| xiii) Lewis b | O | O | O | O | X | O | O | O |
| xiv) Lewis y | O | O | O | O | X | O | O | O |

| | | | | | | | Hepatic |
|---|---|---|---|---|---|---|---|
| | | | | | | | Cancer |
| | Colon Cancer Cell Lines | | | | Ovarian Cancer | | Cell |
| | MDA-435/ | | | | Cell Lines | | Line |
| Carbohydrate Structural Unit | LCC6MDR1 | Colo205 | LS180 | SW1116 | SW626 | PA-1 | HepG2 |
| i) 6-Fucosyl Chitobiose core | 4X | 4X | 4X | 4X | 4X | 4X | 3X |
| ii) 3-Fucosyl Chitobiose core | O | 9X | 16X | X | O | O | O |
| iii) Lewis a | O | 13X | 9X | 4X | X | O | O |
| iv) Lewis x | O | 9X | 9X | 8X | 3X | 3X | T |
| v) 3′-Sialyl T-hapten | 6X | X | 3X | 3X | T | T | 6X |
| vi) 3′-Sulfo Lewis x | O | T | 10X | 5X | X | O | ND^{c} |
| vii) 3′-Sialyl Lewis x | O | T | T | T | O | O | T |
| viii) 3′-Sialyl Lewis a | O | T | T | T | O | O | O |
| ix) T-hapten | T | O | O | O | X | X | T |
| x) 3′-Sulfo T-hapten | 4X | T | 5X | 3X | T | O | ND |
| xi) 3′-Sialyl LacNAc | X | T | T | T | O | O | X |
| xii) 6′-Sialyl LacNAc | X | T | O | O | O | O | 5X |
| xiii) Lewis b | O | T | T | T | T | T | O |
| xiv) Lewis y | O | T | T | T | T | T | O |

^{a}x denotes the lowest concentration that was considered as 1-fold when a comparison of a particular structure was made among all the cancer cell lines studied here.
^{b}T refers to trace amount
^{c}ND—Not Determined

TABLE X

| | | | | | | HEPATIC |
|---|---|---|---|---|---|---|
| | COLON CANCER | | | OVARIAN CANCER | | CANCER |
| ENZYME | Colo205 | LS180 | SW1116 | SW626 | PA-1 | HepG2 |
| Sialyltransferases: | | | | | | |
| α2,3(O)ST | 34.0 | 86.7 | 95.9 | 9.0 | 5.0 | 185.7 |
| α2,3(N)ST | 3.5 | 7.4 | 7.5 | 1.3 | 1.3 | 22.6 |
| α2,6(N)ST | 4.2 | 0 | 0 | 0 | 0 | 362.8 |
| Fucosyltransferases: | | | | | | |
| α1,2-L-FT | 3.8 | 5.5 | 3.4 | 2.7 | 0.9 | 0 |
| α1,3-L-FT | 267.7 | 257.8 | 223.7 | 88.7 | 86.7 | 11.8 |
| α1,4-L-FT | 319.0 | 210.8 | 89.4 | 24.0 | 0.8 | 0 |
| FTV1 | 101.3 | 180.3 | 11.8 | 2.3 | 0.8 | 0 |
| α,6-L-FT | 413.6 | 350.9 | 343.9 | 438.9 | 443.5 | 292.0 |
| FetTA-AsialoGP | 391.7 | 399.7 | 199.0 | 165.4 | 148.9 | 39.6 |
| FetTA-SialoGP | 227.2 | 251.2 | 61.1 | 0 | 0 | 43.6 |
| Ancrod | 77.5 | 75.0 | 16.5 | 0 | 0 | 0 |
| β-Gal/GalNAc-Transferases: | | | | | | |
| GlcNAc: | 297.5 (100.0) | 226.6 (100.0) | 503.4 (100.0) | 478.2 (100.0) | 187.0 (100.0) | 478.3 (100.0) |
| β1,4Gal-T | | | | | | |
| GlcNAc: | 27.6 (9.3) | 73.0 (32.2) | 171.5 (34.1) | 142.8 (29.9) | 75.5 (40.4) | 148.1 (31.0) |
| β1,4GalNAc-T | | | | | | |
| α-GalNAc: | 31.5 | 23.7 | 62.2 | 65.4 | 29.5 | 146.3 |
| β1,3Gal-T | | | | | | |
| Glycan:Sulfotransferases: | | | | | | |
| Galβ1,3GalNAcα-: | 1.3 | 33.4 | 17.3 | 2.5 | 0.8 | ND |
| 3-O-Sulfo-T | | | | | | |
| Galβ1,4GlcNAcβ-: | 6.4 | 216.0 | 92.5 | 15.7 | 0 | ND |
| 3-O-Sulfo-T | | | | | | |
| GlcNAc: 6-O-Sulfo-T | 0.1 | 0.2 | 0.4 | 0 | ND | ND |

Sialyl-, Fucosyl-, β-Galactosyl-, β-N-Acetylgalactosaminyl- and Glycan:3-O-Sulfo-Transferase activities of colon cancer, ovarian cancer, leukemia and hepatic cancer cell lines.
Activity: Incorporation of radioactive sugar or [35ₛ] sulfate [CPM × 10⁻³] catalyzed by 1 mg protein of the cell extract.

TABLE XI
Sialyl-, Fucosyl-, β-Galactosyl-, β-N-Acetylgalactosaminyl- and
Glycan:3-O-Sulfo-Transferase activities in three different tissues.

| | COLON | | BREAST |
|---|---|---|---|
| | TUMOR | | TUMOR |
| ENZYME | CC9338 | | BC9400 |
| Sialyltransferases: | | | |
| α2,3(O)ST | 721.4 | | 283.0 |
| α2,3(N)ST | 7.8 | | 2.3 |
| α2,6(N)ST | 148.0 | | 11.6 |
| Fucosyltransferases: | | | |
| α1,2-L-FT: | | | |
| i) Galβ-O-Bn | 20.6 | | 9.8 |
| ii) Galβ1,3GalNAcα-O—Al | 36.8 | | 5.5 |
| iii) Galβ1,3GalNAcβ1,3Galα- | 49.1 | | 13.7 |
| O-Me | | | |
| iv) D-Fucβ1,3GalNAcβ1,3Galα- | 89.8 | | 16.2 |
| O-Me | | | |
| α1,3-L-FT:2-O- | 540.9 | | 30.5 |
| MeGalβ1,4GlcNAc | | | |
| α1,2-L-FT plus α1,4-L-FT: | | | |
| Galβ1,3GlcNAcβ1,3Galβ-O-Me | 578.9 | | 16.8 |
| α1,4-L-FT: | | | |
| 2-O-MeGalβ1,3GlcNAc | 727.5 | | 4.6 |
| FTVI:GlcNAcβ1,4GlcNAcβ-O-Bn | 139.1 | | 0.3 |
| β-Gal/GalNAc-Transferases: | | | |
| a) β-GlcNAc:β1,4Gal-T | 421.0(100.0%) | | 403.4(100.0%) |
| b) β-GlcNAc:β1,4GalNAc-T | 85.3(20.3%) | | 153.2(38.0%) |
| α-GalNAc:β1,3Gal-T | 102.2(24.3%) | | 38.6(9.6%) |
| a and b by using Other Acceptors: | | | |
| i) GlcNAcβ1,6Manα-O—Al: | a. | 493.4(100.0%) | 443.6(100.0%) |
| | b. | 54.5(11.0%) | 99.2(22.4%) |
| ii) GlcNAcβ1,2Manβ1,6Glcβ- | a. | 277.7 | 70.5 |
| O—Al: | b. | 25.6 | 57.9 |
| iii) GlcNAcβ1,3Galβ-O-Me: | a. | 247.9 | 338.5 |
| | b. | 20.9 | 60.9 |
| iv) GlcNAcβ1,4GlcNAcβ-O-Bn: | a. | 307.6 | 436.7 |
| | b. | 18.9 | 57.8 |

| | | |
|---|---|---|
| Glycan:Sulfotransferases: | | |
| Galβ1,3GalNAcα-:3-O-Sulfo-T | 40.1 | 60.2 |
| Galβ1,4GlcNAcβ-:3-O-Sulfo-T | 181.2 | 5.0 |
| GlcNAc:6-O-Sulfo-T | 52.8 | 69.8 |

Activity: Incorporation of radioactive sugar or [35_{S}] sulfate [CPM × 10⁻³] catalyzed by 1 mg protein of the tissue extract.

TABLE XII
Availability of Novel Acceptors for Enzymatic Studies [^{#}Synthesis of compound
in progress. Bolded sugar residues denote site of glycosyltransferase action].

| Compound | Acceptor | Enzyme |
|---|---|---|
| 1 | Galβ1→4GlcNAcβ1→6(MeO-3Galβ1→3)GalNAcα→OB | ST IV, Sulfo T-4 |
| 2 | MeO-3Galβ1→4GlcNAcβ1→6(Galβ1→3)GalNAcα→OB | ST I, II, Sulfo T-2,3 |
| 3 | Galβ1→4GlcNAcβ1→6(F-3Galβ1→3)GalNAcα→OB | Sulfo T-4 |
| 4 | F-3Galβ1→4GlcNAcβ1→6(Galβ1→3)GalNAcα→OB | Sulfo T-3 |
| 5 | Galβ1→4GlcNAcβ1→6(MeO-4Galβ1→4)GalNAcα→OB | α(1,4) GNT II |
| 6 | (MeO-4Galβ1→4)GlcNAcβ1→6(Galβ1→3)GalNAcα→OB | α(1,4) GNT I |
| 7 | Galβ1→4GlcNAcβ1→6 (F-4Galβ1→3)GalNAcα→OB | α(1,4) GNT II |
| 8 | F-4Galβ1→4GlcNAcβ1→6(Galβ1→3)GalNAcα→OB | α(1,4) GNT I |
| 9 | NeuAcα2→3Galβ1→4GlcNAcβ1→6(MeO-3Galβ1→3)GalNAcα→OB | Gal-6-Sulfo T I |
| 10 | MeO-3Galβ1→4GlcNAcβ1→6(NeuAcα2→3)Galβ1→3GalNAcα→OB | Gal-6-Sulfo T II |
| 11 | Galβ1→4MeO-3GlcNAcβ1→6(GalNAcβ1→3)GalNAcα→OB | α(1,2)-L-FT I |
| 12^{#} | GalNAcβ1→4(MeO-3GlcNAc)β1→6(Galβ1→3)GalNAcα→OB | α(1,2)-L-FT II |
| 13^{#} | SE-6Galβ1→4GlcNAcβ1→6(GalNAc1→3) GalNAcα→OB | α(1,2)-L-FT I for |
| | | Galβ1→4 GlcNAc |
| 14^{#} | SE-6GalNAc1→4GlcNAcβ1→6(SE-6 Gal1→3)GalNAcα→OB | α(1,2) L-FT II |

Abbreviations used herein are as follows:

### AA/CP: Acrylamide Copolymer;

### Al Allyl

BAL British anti-Lewisite

### Bn Benzyl

BSA: Bovine serum albumin;
CPM Counts per minute
FetTA Fetuin triantannery

### FT Fucosyltransferase

GlyCAM-1: Glycosylation-dependent cell adhesion molecule-1;
Globo backbone Galβ1,3GalNAcβ1,3Galα-
GP Glycopeptide molecule-1;

### LacNAc: Galβ1,4GlcNAc;

LacNAc type 1 Galβ1,3 GlcNAc
LacNAc type 2 Galβ1,4GlcNAc

### Lewis^{a} Galβ1,3 (Fucα1,4)GlcNAc

### Lewis^{b} Fucα1,2Galβ1,3(Fucα1,4)GlcNAc

### Lewis^{x} Galβ1,4(Fucα1,3)GlcNAc

### Lewis^{y} Fucα1,2Galβ1,4(Fucα1,3)GlcNAc

### Me Methyl

PAPS 3′-phospho adenosine, 5′-phosphosulfate
PNA: Peanut agglutinin;
PSGL-1: P-selectin glycoprotein ligand-1;
RCA: Ricinus communis agglutinin;
RM: Reaction mixture;
Siglec: Sialic acid-binding lectin of the immunoglobulin super family;

### ST: Sialyltransferase;

### T-hapten: Galβ1,3GalNAcα-;

TLC: Thin layer chromatography;
WGA: Wheat germ agglutinin.

1. A method for determining substrates specific for a transferase enzyme selected from the group consisting of glycosyltransferases and sulfotransferases comprising:
a) selecting a substrate to be tested for specificity for sulfotransferases and for glycosyltransferases, from tri, tetra or penta saccharide compounds containing at least one →GlcNAcβ1 saccharide that may be substituted with MeO—, EtO—, Allyl-O, or N₃— and terminating in →3GalNAcα-OR, →3GlcNAcβ-OR or →3Galα-O—R where R is alkyl, aryl, azido or allyl all of 1 through 8 carbon atoms where R may be substituted with halo, —OH, —SO₃, or —NO₂;
b) combining the substrate separately with each glycosyltransferase or sulfo-transferase in a series, for transfer of a particular glycosyl or sulfo moiety, to a sacccharide, in conjunction with a donor compound for the particular moiety, where the moiety is radio labeled, in a buffered aqueous solution of about pH of about 6 to about 7, to form incubation mixtures;
c) incubating the incubation mixtures at a temperature of from about 18 to about 40 degrees Celcius for from about 30 minutes to about four hours;
d) testing the resulting incubation mixtures of each separate glycosyltransferase or sulfotransferase for reaction product comprising a chemical combination of substrate and particular moiety to determine effectiveness of each separate glycosyltransferase or sulfotransferase in transferring the particular moiety to the substrate; and.
e) comparing the effectiveness of each glycosyltransferase or sulfotransferase in transferring the particular moiety to determine whether a particular glycosyltranferase or sulfotransferase acts to transfer the particular moiety to the substrate when the other glycosyltransferases or sulfotransferases do not do so, thus determining whether the substrate acts specifically with a particular glycosyltransferase or sulfotransferase.

2. The method of claim 1 where the substrate terminates in →3GalNAcα-OR.

3. The method of claim 2 where the substrate also contains at least one of Galβ1, DFucβ1 or an additional →GlcNAcβ1 where the Galβ1, DFucβ1 or additional →GlcNAcβ1 may be substituted with MeO—, EtO—, Allyl-O, or N₃—.

4. The method of claim 1 where the transferase is a glycosyltransferase selected from the group consisting of sialyltransferases, fucosyltransferases, N-acetylglucosaminyl transferases, N-acetylgalactosaminyl transferases, and galactosyltransferases.

5. The method of claim 1 where the incubation temperature is about 35 to about 40° C.

6. The method of claim 1 where the incubation temperature is about 37° C.

7. A method for the specific detection of a specific transferase for a specific moiety selected from the group consisting of sulfotransferases and glycosyltransferases comprising:
a) selecting a substrate specific for the transferase from tri, tetra or penta saccharide compounds containing at least one pre-terminal saccharide that may be substituted with MeO—, EtO—, Allyl-O, or N₃— and terminating in →3GalNAcα-OR, →3Galα-O—R or →3GlcNAcβ-OR where R contains 1 to 12 carbon atoms and is alkyl, aryl or allyl and R may be substituted with halo, —OH, lower alkyl, —SO₃, azido or —NO₂;
b) combining the substrate with a sample to be tested for the specific transferase in conjunction with a donor compound for the particular moiety, in a buffered aqueous solution of about pH 6 to about pH 7, to form an incubation mixture;
c) incubating the incubation mixture at a temperature of from about 18 to about 40 degrees Celcius for from about 30 minutes to about four hours; and
d) testing the resulting incubation mixture for reaction product comprising a chemical combination of substrate and particular moiety to determine the presence of the particular transferase in the sample.

8. The method of claim 7 where the substrate contains at least one GlcNAcβ1 saccharide.

9. The method of claim 7 where the substrate terminates in →3GalNAcα-OR.

10. The method of claim 8 where the substrate, with the exception of saccharide and terminal linkages, is unsubstituted.

11. The method of claim 7 where the substrate also contains at least one of Galβ1, DFucβ1 or an additional →GlcNAcβ1 where the Galβ1, DFucβ1 or additional →GlcNAcβ1 may be substituted with MeO—, EtO—, Allyl-O, or N₃—.

12. The method of claim 11 where, with the exception of saccharide linkages, the Galβ1, DFucβ1 or additional →GlcNAcβ1 are unsubtituted.

13. The method of claim 7 where the incubation temperature is from about 35 to about 40° C.

14. The method of claim 13 where the incubation temperature is about 37° C.

15. The method of claim 7 where steps a) through d) are repeated with different concentrations of substrate to determine the minimum concentration of substrate required to maximize quantity of obtained combination of substrate and particular moiety and the determined minimum concentration is correlated with quantity of the specific transferase in the sample.

16. The method of claim 7 where R is aryl that may be substituted with —OH, halo, or lower alkyl.

17. The method of claim 7 where R is allyl, benzyl, methyl, azido or nitrophenyl.

18-21. (canceled)

22. The method of claim 7 where the substrate is Galβ1→4GlcNAcβ1→6(MeO-4Galβ1→4)GalNAcα→O—R, the specific transferase is α1,4 Nacetylglucosaminyl transferase II (α1,4-GNTII) and the donor compound is uridine diphosphate N-acetylglucosamine (UDP-GlcNAc).

23. The method of claim 7 where the substrate is (MeO-4Galβ1→4)GlcNAcβ1→6(Galβ1→3)GalNAcα→O—R, the specific transferase is α1,4 N-acetylglucosaminyl transferase I (α1,4-GNTI) and the donor compound is uridine diphosphate N-acetylglucosamine (UDP-GlcNAc).

24. The method of claim 7 where the substrate is Galβ1→4GlcNAcβ1→6(F-4Galβ1→3)GalNAcα→O—R, the specific transferase is α1,4 N-acetylglucosaminyl transferase II (α1,4-GNTII) and the donor compound is uridine diphosphate N-acetylglucosamine (UDP-GlcNAc).

25. The method of claim 7 where the substrate is F-4Galβ1→4GlcNAcβ1→6(Galβ1→3)GalNAcα→O—R, the specific transferase is α1 →4 N-acetylglucosaminyl transferase I (α1,4-GNTI) and the donor compound is uridine diphosphate N-acetylglucosamine (UDP-GlcNAc).

26. The method of claim 7 where the substrate is NeuAcα2→3 Gal β1 →4GlcNAcβ1→6(MeO-3 Galβ1→3)GalNAcα→O—R, the specific transferase is the sulfotranserferase, galactose 6 sulfotransferase I (Gal-6-SulfoTI) and the donor compound is 3′ phosphoadenosine 5′ phosphosulfate (PAPS).

27. The method of claim 7 where the substrate is MeO-3Galβ1→4GlcNAcβ1 →6(NeuAcα2→3)Galβ1→3GalNAcα→O—R, the specific transferase is the sulfotranserferase, galactose 6 sulfotransferase II (Gal-6-SulfoTII) and the donor compound is 3′ phosphoadenosine 5′ phosphosulfate (PAPS).

28. The method of claim 7 where the substrate is Galβ1→4MeO-3GlcNAcβ1→6(GalNAcβ1→3)GalNAcα→O—R, the specific transferase is α1,2 L-fucosyltransferase I (α1,2-L-FT I) and the donor compound is guanosine diphosphate fucose (GDP-fucose).

29. The method of claim 7 where the substrate is GalNAcβ1→4(MeO-3GlcNAc)β1→6(Galβ1→3)GalNAcα→O—R, the specific transferase is α1,2 L-fucosyltransferase II (α1,2-L-FT II) and the donor compound is guanosine diphosphate fucose (GDP-fucose).

30. The method of claim 7 where the substrate is GalNAcβ1→4GlcNAcβ1→6(GalNAcβ1→3)GalNAcα→O—R, the specific transferase is α1,3 L-fucosyltransferase (α1,3-L-FT) and the donor compound is guanosine diphosphate fucose (GDP-fucose).

31. The method of claim 7 where the substrate is Galβ1→3(GlcNAcβ1→6) GalNAcα→O—R, the specific transferase is β1→3/4 N-acetyl galactosaminyl transferase (β1→3/4 GalNAc-T) and the donor compound is uridine diphosphate galactosamine (UDP-GalNAc).

32. The method of claim 7 where the substrate is MeO-3Galβ1→3(GlcNAcβ1→6)GalNAcα→O—R, the specific transferase is an αX N-acetyl glucosminyl transferase (αXGlcNAc-T) and the donor compound is uridine diphosphate N-acetylglucosamine (UDP-GlcNAc).

33-39. (canceled)

40. An oligosaccharide compound comprising 3 to 5 linked monosaccharides containing at least one →GlcNAcβ1 saccharide that may be substituted with MeO—, EtO—, Allyl-O, or N₃— and terminating in →3GalNAcα-OR where R is alkyl, aryl or allyl all of 1 through 8 carbon atoms where R may be substituted with halo, —OH, —SO₃, or —NO₂ and further containing at least one of Galβ1, DFucβ1 or an additional →GlcNAcβ1 where the Galβ1, DFucβ1 or additional →GlcNAcβ1 may be substituted with MeO—, EtO—, Allyl-O, or N₃—.

41. The compound of claim 40 selected from the group consisting of Galβ1→4GlcNAcβ1→6(MeO-4Galβ1→3)GalNAcα→O—R; MeO-4Galβ1→4GlcNAcβ1→6(Galβ1→3)GalNAcα→O—R; Galβ1→4GlcNAcβ1→6(F-4Galβ1→3) GalNAcα→O—R; F-4Galβ1→4GlcNAcβ1→6(Galβ1 →3)GalNAcα→O—R; MeO-3 Galβ1→3 (GlcNAcβ1→6)GalNAcα→O—R; Galβ1→3(GlcNAcβ1→6) GalNAcα→O—R; Galβ1→3(GlcNAcβ1→6) GalNAcα→O—R; D-Fucβ1→3GalNAcβ1→3Galα-O-methyl; and GalNAcβ1→4GlcNAcβ1→6(GalNAcβ1→3)GalNAcα→O—R.

42. The compound of claim 41 where R is benzyl.

43. A compound that acts as a specific substrate for a specific glycosyl or sulfo transferase enzyme, said substrate being selected from the group consisting of: Galβ1 →4GlcNAcβ1→6(MeO-4Galβ1→4)GalNAcα→O—R; MeO-Galβ1→4GlcNAcβ1→6(Galβ1→3)GalNAcα→O—R; Galβ1→4GlcNAcβ1→6(F-4Galβ1→3) GalNAcα→O—R; and F-4Galβ1→4GlcNAcβ1→6(Galβ1→3)GalNAcα→O—R where R contains 1 to 12 carbon atoms and is alkyl, aryl, azido, or allyl, that may be substituted with halo, —OH, —SO₃, or —NO₂.

44. The method of claim 7 where the specific substrate is selected from the group consisting of Galβ1→4GlcNAcβ1→6(MeO-3Gal β1→3)GalNAcα→O—R; Galβ1→4GlcNAcβ1→6(F-3 Galβ1→3)GalNAcα→O—R; 2-O-MeGalβ1→4GlcNAcβ3-O-benzyl and 4-O-MeGalβ1→4GlcNAcβ-O-benzyl; the specific transferase is sialyl transferase IV (ST IV) and the donor compound is cytidine-5′ monophospate-Nacetylneuraminic acid (CMP-sialic acid).

45. The method of claim 7 where the specific substrate is selected from the group consisting of: MeO-Galβ1→4GlcNAcβ1→6(Galβ1→6)GalNAcα→O—R, F-3 Galβ1→4GlcNAcβ1→6(Galβ1→3) GalNAcα→O—R, D-Fucβ1→3GalNAcβ1→3Galα-O-methyl, 2-O-MeGalβ1→4GlcNAcβ-O-benzyl, the specific transferase is sialyl transferase I/II (ST I/II) and the donor compound is cytidine-5′ monophospate-Nacetylneuraminic acid (CMP-sialic acid).
